(19) United States
(12) Patent Application Publication
(10) Pub. No.: US 2017/0273970 A1
(43) Pub. Date: Sep. 28, 2017

(54) Treatment and/or prophylaxis of TSPO mediated diseases and/or disorders

(71) Applicant: GRÜNENTHAL GMBH, Aachen (DE)

(72) Inventors: Petra BLOMS-FUNKE, Würselen (DE); Gregor Bahrenberg, Monschau-Konzen (DE); Wolfgang Schröder, Aachen (DE); Sven Kühnert, Düren (DE); Simon Lucas, Wolfsgraben (AT)

(73) Assignee: GRÜNENTHAL GMBH, Aachen (DE)

(21) Appl. No.: 15/622,292

(22) Filed: Jun. 14, 2017

Related U.S. Application Data
(63) Continuation-in-part of application No. 14/434,943, filed on Apr. 10, 2015.

(30) Foreign Application Priority Data
Oct. 11, 2012 (EP) 12007118.8

(51) Int. Cl. A61K 31/47

(52) U.S. Cl. CPC A61K 31/47

(57) ABSTRACT

The invention relates to certain compounds for the use in the treatment and/or prophylaxis of diseases and/or disorders that are, at least partially, mediated by TSPO activity.

This application is a continuation-in-part of U.S. Ser. No. 14/434,943, filed Apr. 10, 2015, now allowed, which is a 371 of International Patent Application No. PCT/EP2013/003064, filed Oct. 11, 2013, which claims foreign priority benefit under 35 U.S.C. §119 of the European Patent Application No. 12007118.8, filed Oct. 11, 2012, the disclosures of which patent applications are incorporated herein by reference.

The invention relates to certain compounds for the use in the treatment and/or prophylaxis of diseases and/or disorders that are, at least partially, mediated by TSPO activity.

The translocator protein (18 kDa) (TSPO), also known as peripheral-type benzodiazepine receptor, peripheral BZD receptor or mitochondrial benzodiazepine receptor, is a five transmembrane domain protein that is localized primarily in the outer mitochondrial membrane and expressed predominantly in steroid-synthesizing tissues, including the brain. TSPO is involved in the translocation of cholesterol from the outer to the inner mitochondrial membrane, which is the rate-limiting step in the synthesis of steroids and one of the most well-characterized functions of this protein.

Radiolabeled TSPO ligands are used as neuroimaging agents and are known as important diagnostic tools. TSPO also seems to be a sensitive biomarker of brain damage and neurodegeneration. The binding of labeled TSPO ligands can be visualized and quantified by in vivo imaging techniques such as positron emission tomography (PET) and single photon emission computed tomography (SPECT). Altered TSPO expression has been shown in numerous neurological and psychiatric disorders.

Therefore, the modulation of the activity of this protein provides an option for treatment of diseases related to disturbed cholesterol metabolism and steroidogenesis, but also aptotosis and cell proliferation.

Furthermore, its potential use for neuroprotection, limiting immunological disorders, inflammatory diseases including neuroinflammation and arthritis, encephalomyelitis, irritable bowel syndrome, promoting regeneration, and treating dysfunctions of the nervous system has been envisaged (Rupprecht et al., Nature Reviews, 2010, Vol. 9, 971).

TSPO ligands also provide therapeutic potential in neuroregeneration, and anxiety, particularly for the treatment of neurodegenerative disorders such as Alzheimer's disease, head trauma, frontotemporal dementia, multiple sclerosis, Huntington's disease, amyotrophic lateral sclerosis, Morbus Parkinson, cerebrovascular disorders, cerebral ischemia, stroke, tumours including brain tumours, and neuro-degenerative diseases related to peripheral nervous system lesions and neuropathologies.

Various TSPO ligands have been shown to exert acute anxiolytic/anticonflict activity in rodents and humans. Therefore, the modulation of TSPO activity additionally offers treatment options for psychiatric disorders such as anxiety disorders including panic attacks, stress disorders including posttraumatic stress disorder, depression including major depressive disorders, dysthymia, angina pectoris (Rupprecht et al., Nature Reviews, 2010, Vol. 9, 971).

It was therefore an object of the invention to provide compounds having advantages over the compounds known for TSPO activity. These compounds should be suitable in particular as pharmacological active ingredients in pharmaceutical compositions, preferably in pharmaceutical compositions for the treatment and/or prophylaxis of disorders and/or diseases which are, at least partially, mediated by TPSO activity.

That object is achieved by the subject-matter of the patent claims. It has been found, surprisingly, that compounds of the general formula (I) given below are suitable for the use in the treatment and/or prophylaxis of disorders and/or diseases which are, at least partially, mediated by TPSO activity.

Compounds according to general formula (I) are known and have been described for their activity on KCNQ2/3 K⁺ channels (WO 2010/102809 A1; WO 2010/102811 A1; WO 2012/025236 A1; WO 2012/025237 A1; WO 2012/025238 A1; WO 2012/025239 A1; WO 2012/0052167 A1).

No hint for modulation of TSPO activity and the treatment and/or prophylaxis of disorders and/or diseases, which are, at least partially, mediated by TPSO activity, however, is provided in the prior art.

The first aspect of the present invention therefore relates to a compound of general formula (I),

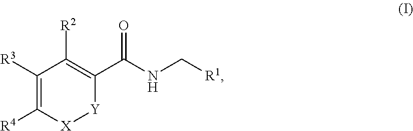

for the use in the treatment and/or prophylaxis of disorders and/or diseases which are mediated, at least in part, by TSPO (translocator protein 18 kDa),

wherein
X—Y represents N(R⁵)—C(═O) or N(R⁵)—C(═S) or N═C(R⁶)
R¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R² represents H; F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁴ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; or represents N(R⁷)(R⁸),
   wherein
   R⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
      on the condition that if R⁷ denotes a 3 to 10 membered heterocycloaliphatic residue or a heteroaryl, the 3 to 10 membered heterocycloaliphatic residue or the heteroaryl is linked via a carbon atom;
   R⁸ denotes H or a C₁₋₁₀-aliphatic residue, unsubstituted or mono- or polysubstituted; or
   R⁷ and R⁸ form together with the nitrogen atom connecting them a 3 to 10 membered heterocycloaliphatic residue, unsubstituted or mono- or polysubstituted;
or R³ and R⁴ represent CH═CH—CH═CH,
   wherein one or more H-Atoms independently of one another may be replaced by one a radical selected from the group consisting of F; Cl; Br; I; CN; CF₃; C(═O)H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂—C₁₋₄-aliphatic residue, a N(C₁₋₄ aliphatic residue)-C(═O)—C₁₋₄ aliphatic residue, or a N(C₁₋₄ aliphatic residue)-S(═O)₂—C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁵ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁵ denotes a 3 to 10 membered heterocycloaliphatic residue, the 3 to 10 membered heterocycloaliphatic residue is linked via a carbon atom,
R⁶ represents a C₂₋₁₀-aliphatic residue or a C₂₋₁₀-heteroaliphatic residue, in each case unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; or
   denotes S—R¹¹, O—R¹² or N(R⁹R¹⁰),
      wherein
      R¹¹ and R¹² in each case represent a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group or a C₂₋₈-heteroaliphatic group, which in turn may be unsubstituted or mono- or polysubstituted;
         on the condition that if R¹¹ or R¹² denotes a 3 to 10 membered heterocycloaliphatic residue, the 3 to 10 membered heterocycloaliphatic residue is linked via a carbon atom,
      R⁹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁹ denotes a 3 to 10 membered heterocycloaliphatic residue, the 3 to 10 membered heterocycloaliphatic residue is linked via a carbon atom;
      R¹⁰ denotes a C₁₋₁₀-aliphatic residue, unsubstituted or mono- or polysubstituted; or
      R⁹ and R¹⁰ form together with the nitrogen atom connecting them a 3 to 10 membered heterocycloaliphatic residue, unsubstituted or mono- or polysubstituted;
   in the form of the free compounds, the racemate, the enantiomers, diastereomers, mixtures of the enantiomers or diastereomers in any mixing ratio, or of an individual enantiomer or diastereomer, or in the form of the salts of physiologically acceptable acids or bases.

The term “mono- or polysubstituted” with respect to an “aliphatic group” and an “aliphatic residue”, “alkyl group”, “alkyl residue”, “heteroaliphatic group”, “heteroalipahtic residue”, “heteroalkyl group”, “heteroalkyl residue”, “heterocyloaliphatic group”, “heterocycloalipahtic residue”, “heterocycloalkyl group”, “heterocycloalkyl residue”, “cycloalipahtic residue” and “cycloalkyl” within the scope of the present invention is the substitution of one or more hydrogen atoms, in each case independently of one another, by F; Cl; Br; I; NO₂; CF₃; CN; ═O; C₁₋₈-alkyl; C₂₋₈-heteroalkyl; aryl; heteroaryl; C₃₋₁₀-cycloalkyl; heterocyclyl; C₁₋₈-alkyl- or C₂₋₈-heteroalkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂; NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; NH—C(═O)-aryl; NH—C(═O)-heteroaryl; NH—S(═O)₂—C₁₋₈-alkyl; NH—S(═O)₂-aryl; NH—S(═O)₂-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; and S(═O)₂—NH—C₁₋₈-heteroaryl.

The term “mono- or polysubstituted” with respect to “aryl” and a “heteroaryl” within the scope of the present invention is the substitution of one or more hydrogen atoms, in each case independently of one another, by F; Cl; Br; I; NO₂; CF₃; CN; C₁₋₈-alkyl; C₂₋₈-heteroalkyl; aryl; heteroaryl; C₃₋₁₀-cycloalkyl; heterocyclyl; C₁₋₈-alkyl- or C₂₋₈-heteroalkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂, NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; S(═O)₂—NH—C₁₋₈-heteroaryl;

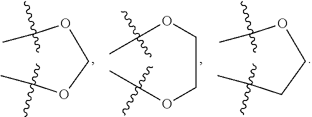 [SMILES: *OCO* ; *OCCO* ; *CCO*]

Within the scope of this invention, the terms “alkyl” and “aliphatic residue” or “aliphatic group” or “C₁₋₁₀-alkyl”, “C₁₋₈-alkyl”, “C₁₋₆-alkyl”, “C₁₋₄-alkyl”, “C₁₋₂-alkyl” and “C₂₋₆-alkyl” include acyclic saturated or unsaturated aliphatic hydrocarbon radicals, which can be branched or unbranched as well as unsubstituted or mono- or poly-substituted, having from 1 to 10 or from 1 to 8 or from to 6 or from 1 to 4 or from 1 to 2 or from 2 to 6 carbon atoms, that is to say C₁₋₁₀-alkanyls, C₂₋₁₀-alkenyls and C₂₋₁₀-alkynyls or C₁₋₈-alkanyls, C₂₋₈-alkenyls and C₂₋₈-alkynyls or C₁₋₆-alkanyls, C₂₋₆-alkenyls and C₂₋₆-alkynyls or C₁₋₄-alkanyls, C₂₋₄-alkenyls and C₂₋₄-alkynyls or C₁₋₂-alkanyls, C₂-alkenyls and C₂-alkynyls or C₂₋₆-alkanyls, C₂₋₆-alkenyls and C₂₋₆-alkynyls. Alkenyls contain at least one C—C double bond and alkynyls contain at least one C—C triple bond. Alkyl is preferably selected from the group comprising methyl, ethyl, n-propyl, 2-propyl, n-butyl, isobutyl, sec-butyl, tert-butyl, n-pentyl, isopentyl, neopentyl, n-hexyl, n-heptyl, n-octyl, n-nonyl, n-decyl, ethenyl (vinyl), ethynyl, propenyl (—CH₂CH═CH₂, —CH═CH—CH₃, —C(═CH₂)—CH₃), propynyl (—CH—C≡CH, —C≡C—CH₃), butenyl, butynyl, pentenyl, pentynyl, hexenyl and hexynyl, heptenyl, heptynyl, octenyl, octynyl, nonenyl, nonynyl, decenyl and decynyl.

Within the scope of this invention, the terms “heteroalkyl” or “C₂₋₁₀-heteroalkyl” and “C₂₋₈-heteroalkyl” or “heteroaliphatic” or “C₂₋₁₀-heteroaliphatic” and “C₂₋₈-heteroaliphatic” include acyclic aliphatic saturated or unsaturated hydrocarbon radicals having from 2 to 10 carbon atoms, that is to say C₂₋₁₀-heteroalkanyls, C₂₋₁₀-heteroalkenyls and C₂₋₁₀-heteroalkynyls, or having from 2 to 8 carbon atoms, that is to say C₂₋₈-heteroalkanyls, C₂₋₈-heteroalkenyls and C₂₋₈-heteroalkynyls, which in each case can be branched or unbranched as well as unsubstituted or mono- or poly-substituted and in which at least one carbon atom, optionally also two or three carbon atoms, have been replaced by a heteroatom or heteroatom group in each case selected independently of one another from the group consisting of O, N, NH and N(C₁₋₈-alkyl), preferably N(CH₃), wherein the initial carbon atom of a C₂₋₁₀-heteroalkyl or of a C₂₋₈-heteroalkyl, via which the C₂₋₁₀-heteroalkyl or C₂₋₈-heteroalkyl is bonded to the respective general structure of higher order, cannot be replaced by a heteroatom or heteroatom group and adjacent carbon atoms cannot simultaneously be replaced by a heteroatom or heteroatom group. The heteroatom groups NH and N(C₁₋₈-alkyl) of the heteroalkyl can optionally be mono- or poly-substituted. C₂₋₁₀-Heteroalkenyls and C₂₋₈-heteroalkenyls contain at least one C—C or C—N double bond and C₂₋₁₀-heteroalkynyls and C₂₋₈-heteroalkynyls contain at least one C—C triple bond. Heteroalkyl is preferably selected from the group comprising —CH₂—O—CH₃, —CH₂—CH₂—O—CH₃, —CH₂—CH₂—O—CH₂—CH₃, —CH₂—CH₂—O—CH₂—CH₂—O—CH₃, —CH═CH—O—CH₃, —CH═CH—O—CH₂—CH₃, ═CH—O—CH₃, ═CH—O—CH₂—CH₃, ═CH—CH₂—O—CH₂—CH₃, ═CH—CH₂—O—CH₃, —CH₂—NH—CH₃, —CH₂—CH₂—NH—CH₃, —CH₂—CH₂—NH—CH₂—CH₃, —CH₂—CH₂—NH—CH₂—CH₂—NH—CH₃, —CH═CH—NH—CH₃, —CH═CH—NH—CH₂—CH₃, —CH═CH—N(CH₃)—CH₂—CH₃, ═CH—NH—CH₃, ═CH—NH—CH₂—CH₃, ═CH—CH₂—NH—CH₂—CH₃, ═CH—CH₂—NH—CH₃, —CH₂—N(CH₃)—CH₃, —CH₂—CH₂—N(CH₃)—CH₃, —CH₂—CH₂—N(CH₃)—CH₂—CH₃, —CH₂—CH₂—N(CH₃)—CH₂—CH₂—N(CH₃)—CH₃, CH₂—CH₂—NH—CH₂—CH₂—O—CH₃, CH₂—CH₂—O—CH₂—CH₂—NH—CH₃, CH₂—CH₂—N(CH₃)—CH₂—CH₂—O—CH₃, CH₂—CH₂—O—CH₂—CH₂—N(CH₃)—CH₃, CH₂—NH—CH₂—O—CH₃, CH₂—O—CH₂—NH—CH₃, CH₂—N(CH₃)—CH₂—O—CH₃, CH₂—O—CH₂—N(CH₃)—CH₃, —CH═CH—N(CH₃)—CH₃, ═CH—N(CH₃)—CH₃, ═CH—N(CH₃)—CH₂—CH₃, ═CH—CH₂—N(CH₃)—CH₂—CH₃, ═CHCH₂—N(CH₃)—CH₃, —CH₂CH₂═N(CH₃) and —CH₂═N(CH₃).

For the purposes of this invention, the terms “cycloalkyl” or “C₃₋₁₀-cycloalkyl”, “C₃₋₇-cycloalkyl” and “C₃₋₈-cycloalkyl” or “cycloaliphatic” denote cyclic aliphatic hydrocarbons having 3, 4, 5, 6, 7, 8, 9 or 10 carbon atoms or having 3, 4, 5, 6 or 7 carbon atoms or having 3, 4, 5, 6, 7 or 8 carbon atoms, wherein the hydrocarbons can be saturated or unsaturated (but not aromatic), unsubstituted or mono- or poly-substituted. The bonding of the cycloalkyl to the general structure of higher order can take place via any desired and possible ring member of the cycloalkyl radical. The cycloalkyl radicals can also be fused with further saturated, (partially) unsaturated, (hetero)cyclic, aromatic or heteroaromatic ring systems, that is to say with cycloalkyl, heterocyclyl, aryl or heteroaryl, which can themselves be unsubstituted or mono- or poly-substituted. The cycloalkyl radicals can further be bridged one or more times, as, for example, in the case of adamantyl, bicyclo[2.2.1]heptyl or bicyclo[2.2.2]octyl. Cycloalkyl is preferably selected from the group comprising cyclopropyl, cyclobutyl, cyclopentyl, cyclohexyl, cycloheptyl, cyclooctyl, cyclononyl, cyclodecyl, adamantyl,

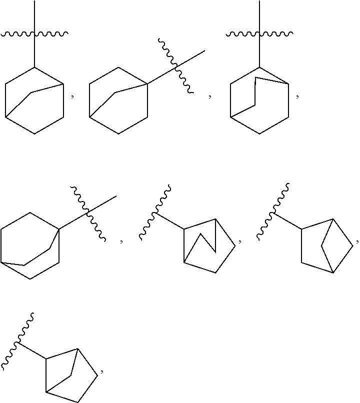

cyclopentenyl, cyclohexenyl, cycloheptenyl and cyclooctenyl.

The term “heterocyclyl” or “heterocycloalkyl” or “heterocycloaliphatic” includes aliphatic saturated or unsaturated (but not aromatic) cycloalkyls having from three to ten, that is to say 3, 4, 5, 6, 7, 8, 9 or 10, ring members, in which at least one carbon atom, optionally also two or three carbon atoms, has been replaced by a heteroatom or heteroatom group in each case selected independently of one another from the group consisting of O, S, N, NH and N(C₁₋₈-alkyl), preferably N(CH₃), wherein the ring members can be unsubstituted or mono- or poly-substituted. The bonding of the heterocyclyl to the general structure of higher order can take place via any desired and possible ring member of the heterocyclyl radical. The heterocyclyl radicals can also be fused with further saturated, (partially) unsaturated (hetero)cyclic or aromatic or heteroaromatic ring systems, that is to say with cycloalkyl, heterocyclyl, aryl or heteroaryl, which can themselves be unsubstituted or mono- or poly-substituted. Heterocyclyl radicals are preferably selected from the group comprising azetidinyl, aziridinyl, azepanyl, azocanyl, diazepanyl, dithiolanyl, dihydroquinolinyl, dihydropyrrolyl, dioxanyl, dioxolanyl, dihydroindenyl, dihydropyridinyl, dihydrofuranyl, dihydroisoquinolinyl, dihydroindolinyl, dihydroisoindolyl, imidazolidinyl, isoxazolidinyl, morpholinyl, oxiranyl, oxetanyl, pyrrolidinyl, piperazinyl, piperidinyl, pyrazolidinyl, pyranyl, tetrahydropyrrolyl, tetrahydropyranyl, tetrahydroquinolinyl, tetrahydroisoquinolinyl, tetrahydroindolinyl, tetrahydrofuranyl, tetrahydropyridinyl, tetrahydrothiophenyl, tetrahydropyridoindolyl, tetrahydronaphthyl, tetrahydrocarbolinyl, tetrahydroisoxazolopyridinyl, thiazolidinyl and thiomorpholinyl.

Within the scope of this invention, the term “aryl” denotes aromatic hydrocarbons having up to 14 ring members, inter alia phenyls and naphthyls. Each aryl radical can be unsubstituted or mono- or poly-substituted, it being possible for the aryl substituents to be identical or different and to be in any desired and possible position of the aryl. The aryl can be bonded to the general structure of higher order via any desired and possible ring member of the aryl radical. The aryl radicals can also be fused with further saturated, (partially) unsaturated, (hetero)cyclic, aromatic or heteroaromatic ring systems, that is to say with cycloalkyl, heterocyclyl, aryl or heteroaryl, which can themselves be unsubstituted or mono- or poly-substituted. Examples of fused aryl radicals are benzodioxolanyl and benzodioxanyl. Aryl is preferably selected from the group containing phenyl, 1-naphthyl and 2-naphthyl, each of which can be unsubstituted or mono- or poly-substituted. A particularly preferred aryl is phenyl, unsubstituted or mono- or poly-substituted.

The term “heteroaryl” denotes a 5- or 6-membered cyclic aromatic radical which contains at least 1 heteroatom, optionally also 2, 3, 4 or 5 heteroatoms, wherein the heteroatoms are in each case selected independently of one another from the group S, N and O and the heteroaryl radical can be unsubstituted or mono- or poly-substituted; in the case of substitution on the heteroaryl, the substituents can be identical or different and can be in any desired and possible position of the heteroaryl. Bonding to the general structure of higher order can take place via any desired and possible ring member of the heteroaryl radical. The heteroaryl can also be part of a bi- or poly-cyclic system having up to 14 ring members, wherein the ring system can be formed with further saturated, (partially) unsaturated, (hetero)cyclic or aromatic or heteroaromatic rings, that is to say with cycloalkyl, heterocyclyl, aryl or heteroaryl, which can themselves be unsubstituted or mono- or poly-substituted. It is preferred for the heteroaryl radical to be selected from the group comprising benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazolyl, isothiazolyl, indolyl, naphthyridinyl, oxazolyl, oxadiazolyl, phenazinyl, phenolthiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl and triazinyl. Furyl, pyridyl and thienyl are particularly preferred.

Within the scope of the invention, the expressions “C₁₋₄-alkyl- or C₁₋₈-alkyl-bridged aryl, heteroaryl, heterocyclyl or cycloalkyl” mean that C₁₋₄-alkyl or C₁₋₈-alkyl and aryl or heteroaryl or heterocyclyl or cycloalkyl have the meanings defined above and the aryl or heteroaryl or heterocyclyl or cycloalkyl radical is bonded to the general structure of higher order via a C₁₋₄-alkyl or C₁₋₈-alkyl group. The alkyl chain can in all cases be saturated or unsaturated, branched or unbranched, unsubstituted or mono- or poly-substituted. C₁₋₄-Alkyl or C₁₋₈-alkyl is preferably selected from the group comprising —CH₂—, —CH₂CH₂—, —CH(CH₃)—, —CH₂CH₂CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂—, —CH₂—(CH₂)₄—CH₂—, —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH═CH═CH—CH₂—CH₂—, —CH═CH₂—CH—CH═CH₂—, —C≡C—, —C≡C—CH₂—, —C≡C—CH₂—CH₂—, —C≡C—CH(CH₃)—, —CH₂—C≡C—CH₂—, —C≡C—C≡C—, —C≡C—C(CH₃)₂—, —C≡C—CH₂—CH₂—CH₂—, —CH₂—C≡C—CH₂—CH₂—, —C≡C—C≡C—CH₂— and —C≡C—CH₂—C≡C—.

Within the scope of the invention, the expressions “C₂₋₈-heteroalkyl-bridged aryl, heteroaryl, heterocyclyl or cycloalkyl” mean that C₂₋₈-heteroalkyl and aryl or heteroaryl or heterocyclyl or cycloalkyl have the meanings defined above and the aryl or heteroaryl or heterocyclyl or cycloalkyl radical is bonded to the general structure of higher order via a C₂₋₈-heteroalkyl group. The heteroalkyl chain can in all cases be saturated or unsaturated, branched or unbranched, unsubstituted or mono- or poly-substituted. If a terminal carbon atom of the C₂₋₈-heteroalkyl group has been replaced by a heteroatom or heteroatom group, then the bonding of a heteroaryl or heterocyclyl to the heteroatom or heteroatom group of the C₂₋₈-heteroalkyl always takes place via a carbon atom of the heteroaryl or heterocyclyl. The terminal carbon atom is understood as being the carbon atom within the C₂₋₈-heteroalkyl that is furthest in the chain from the general structure of higher order. If the terminal carbon atom of a C₂₋₈-heteroalkyl has been replaced, for example, by an N(CH₃) group, that group is located within the C₂₋₈-heteroalkyl furthest from the general structure of higher order and is bonded to the aryl or heteroaryl or heterocyclyl or cycloalkyl radical. C₂₋₈-Heteroalkyl is preferably selected from the group comprising —CH₂—NH—, —CH₂—N(CH₃)—, —CH₂—O—, —CH₂—CH₂—NH—, —CH₂—CH₂—N(CH₃)—, —CH₂—CH₂—O—, —CH₂—CH₂—CH₂—NH—, —CH₂—CH₂—CH₂—N(CH₃)—, —CH₂—CH₂—CH₂—O—, —CH₂—O—CH₂—, —CH₂—CH₂—O—CH₂—, —CH₂—CH₂—O—CH₂—CH₂—, —CH₂—CH₂—O—CH₂—CH₂—O—CH₂—, —CH═CH—O—CH₂—, —CH═CH—O—CH₂—CH₂—, ═CH—O—CH₂—, ═CH—O—CH₂—CH₂—, ═CH—CH₂—O—CH₂—CH₂—, ═CH—CH₂—O—CH₂—, —CH₂—NH—CH₂—, —CH₂—CH₂—NH—CH₂—, —CH₂—CH₂—NH—CH₂—CH₂—, —CH₂—CH₂—NH—CH₂—CH₂—NH—CH₂, —CH═CH—NH—CH₂—, —CH═CH—NH—CH₂—CH₂—, —CH═CH—N(CH₃)—CH₂—CH₂—, ═CH—NH—CH₂—, ═CH—NH—CH₂—CH₂—, ═CH—CH₂—NH—CH₂—CH₂—, ═CH—CH₂—NH—CH₂—, —CH₂—N(CH₃)—CH₂—, —CH₂—CH₂—N(CH₃)—CH₂—, —CH₂—CH₂—N(CH₃)—CH₂—CH₂—, —CH₂—CH₂—N(CH₃)—CH₂—CH₂—N(CH₃)—CH₂—, CH₂—CH₂—NH—CH₂—CH₂—O—CH₂—, CH₂—CH₂—O—CH₂—CH₂—NH—CH₂—, CH₂—CH₂—N(CH₃)—CH₂—CH₂—O—CH₂—, CH₂—CH₂—O—CH₂—CH₂—N(CH₃)—CH₂—, CH₂—NH—CH₂—O—CH₂—, CH₂—O—CH₂—NH—CH₂—, CH₂—N(CH₃)—CH₂—O—CH₂—, CH₂—O—CH₂—N(CH₃)—CH₂—, —CH═CH—N(CH₃)—CH₂—, ═CH—N(CH₃)—CH₂—, ═CH—N(CH₃)—CH₂—CH₂—, ═CH—CH₂—N(CH₃)—CH₂—CH₂— and ═CH—CH₂—N(CH₃)—CH₂—.

In connection with “alkyl”, “heteroalkyl”, “heterocyclyl” and “cycloalkyl”, the expression “mono- or poly-substituted” is understood as meaning within the scope of this invention the substitution of one or more hydrogen atoms one or more times, for example two, three or four times, in each case independently of one another, by substituents selected from the group comprising F; Cl; Br; I; NO₂; CF₃; CN; ═O; C₁₋₈-alkyl; C₂₋₈-heteroalkyl; aryl; heteroaryl; C₃₋₁₀-cycloalkyl; heterocyclyl; C₁₋₈-alkyl- or C₂₋₈-heteroalkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂, NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; and S(═O)₂—NH—C₁₋₈-heteroaryl; wherein polysubstituted radicals are to be understood as being radicals that are substituted several times, for example two, three or four times, either on different atoms or on the same atom, for example three times on the same carbon atom, as in the case of CF₃ or CH₂CF₃, or at different places, as in the case of CH(OH)—CH═CH—CHCl₂. A substituent can itself optionally be mono- or poly-substituted. Polysubstitution can take place with the same or with different substituents.

Preferred “alkyl”, “heteroalkyl”, “heterocyclyl” and “cycloalkyl” substituents are selected from the group comprising F; Cl; Br; I; NO₂; CH₂CF₃; CF₃; CN; C₁₋₈-alkyl; C₂₋₈-heteroalkyl; phenyl; naphthyl; pyridyl; thienyl; furyl; C₃₋₁₀-cycloalkyl; heterocyclyl; C₁₋₈-alkyl- or C₂₋₈-heteroalkyl-bridged phenyl, naphthyl, pyridyl, thienyl, furyl, C₃₋₁₀-cycloalkyl or heterocyclyl; CHO; C(═O)C₁₋₈-alkyl; CO₂H; C(═O)O—C₁₋₈-alkyl; CONH₂; C(═O)NH—C₁₋₈-alkyl; C(═O)N(C₁₋₈-alkyl)₂; OH; ═O; O—C₁₋₈-alkyl; OCF₃; O—(C₁₋₈-alkyl)-OH; O—(C₁₋₈-alkyl)-O—C₁₋₈-alkyl; O-benzyl; O-phenyl; O-heteroaryl; O—C(═O)C₁₋₈-alkyl; NH₂, NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; SH; S—C₁₋₈-alkyl; SCF₃; S-benzyl; S-phenyl; S-heteroaryl; S(═O)₂C₁₋₈-alkyl; S(═O)₂OH; S(═O)₂O—C₁₋₈-alkyl; S(═O)₂—NH—C₁₋₈-alkyl.

In connection with “aryl” and “heteroaryl”, “mono- or poly-substituted” is understood within the scope of this invention as meaning the substitution of one or more hydrogen atoms of the ring system one or more times, for example two, three or four times, in each case independently of one another, by substituents selected from the group comprising F; Cl; Br; I; NO₂; CF₃; CN; C₁₋₈-alkyl; C₂₋₈-heteroalkyl; aryl; heteroaryl; C₃₋₁₀-cycloalkyl; heterocyclyl; C₁₋₈-alkyl- or C₂₋₈-heteroalkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂, NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; S(═O)₂—NH—C₁₋₈-heteroaryl; on one atom or optionally on different atoms, wherein a substituent can itself optionally be mono- or poly-substituted. Polysubstitution is carried out with the same or with different substituents.

Preferred “aryl” and “heteroaryl” substituents are F; Cl; Br; I; NO₂; CH₂CF₃; CF₃; CN; C₁₋₈-alkyl; C₂₋₈-heteroalkyl; phenyl; naphthyl; pyridyl; thienyl; furyl; C₃₋₁₀-cycloalkyl; heterocyclyl; C₁₋₈-alkyl- or C₂₋₈-heteroalkyl-bridged phenyl, naphthyl, pyridyl, thienyl, furyl, C₃₋₁₀-cycloalkyl or heterocyclyl; CHO; C(═O)C₁₋₈-alkyl; CO₂H; C(═O)O—C₁₋₈-alkyl; CONH₂; C(═O)NH—C₁₋₈-alkyl; C(═O)N(C₁₋₈-alkyl)₂; OH; O—C₁₋₈-alkyl; OCF₃; O—(C₁₋₈-alkyl)-OH; O—(C₁₋₈-alkyl)-O—C₁₋₈-alkyl; O-benzyl; O-phenyl; O-heteroaryl; O—C(═O)C₁₋₈-alkyl; NH₂, NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; SH; S—C₁₋₈-alkyl; SCF₃; S-benzyl; S-phenyl; S-heteroaryl; S(═O)₂C₁₋₈-alkyl; S(═O)₂OH; S(═O)₂O—C₁₋₈-alkyl; S(═O)₂—NH—C₁₋₈-alkyl.

The compounds according to the invention are defined by substituents, for example by R¹, R² and R³ (1st generation substituents), which are themselves optionally substituted (2nd generation substituents). Depending on the definition, these substituents of the substituents can in turn themselves be substituted (3rd generation substituents). If, for example, R¹=aryl (1st generation substituent), aryl can itself be substituted, for example by C₁₋₈-alkyl (2nd generation substituent). This yields the functional group aryl-C₁₋₈-alkyl. C₁₋₈-Alkyl can then in turn itself be substituted, for example by CI (3rd generation substituent). Overall, this then yields the functional group aryl-C₁₋₈-alkyl-Cl.

In a preferred embodiment, however, the 3rd generation substituents cannot themselves be substituted, that is to say there are no 4th generation substituents.

In another preferred embodiment, the 2nd generation substituents cannot themselves be substituted, that is to say there are not even any 3rd generation substituents. In other words, in this embodiment, for example in the case of the general formula (1), the functional groups for R¹ to R¹³ can in each case optionally be substituted, but the substituents in each case cannot themselves be substituted.

In some cases, the compounds according to the invention are defined by substituents which are or carry an aryl or heteroaryl radical, in each case unsubstituted or mono- or poly-substituted, or which, together with the carbon atom(s) or heteroatom(s) joining them as ring member(s), form a ring, for example an aryl or heteroaryl, in each case unsubstituted or mono- or poly-substituted. Both these aryl or heteroaryl radicals and the aromatic ring systems so formed can optionally be fused with C₃₋₁₀-cycloalkyl or heterocyclyl, in each case saturated or unsaturated, that is to say with a C₃₋₁₀-cycloalkyl such as cyclopentyl or with a heterocyclyl such as morpholinyl, it being possible for the C₃₋₁₀-cycloalkyl or heterocyclyl radicals so fused to be unsubstituted or mono- or poly-substituted.

In some cases, the compounds according to the invention are defined by substituents which are or carry a C₃₋₁₀-cycloalkyl or heterocyclyl radical, in each case unsubstituted or mono- or poly-substituted, or which, together with the carbon atom(s) or heteroatom(s) joining them as ring member(s), form a ring, for example a C₃₋₁₀-cycloalkyl or heterocyclyl, in each case unsubstituted or mono- or poly-substituted. Both these C₃₋₁₀-cycloalkyl or heterocyclyl radicals and the aliphatic ring systems formed can optionally be fused with aryl or heteroaryl, that is to say with an aryl such as phenyl or with a heteroaryl such as pyridyl, it being possible for the aryl or heteroaryl radicals so fused to be unsubstituted or mono- or poly-substituted.

The expression “salt formed with a physiologically acceptable acid” is understood within the scope of this invention as meaning salts of the active ingredient in question with inorganic or organic acids that are physiologically acceptable—in particular when used in humans and/or mammals. The hydrochloride is particularly preferred. Examples of physiologically acceptable acids are: hydrochloric acid, hydrobromic acid, sulfuric acid, methanesulfonic acid, formic acid, acetic acid, oxalic acid, succinic acid, tartaric acid, mandelic acid, fumaric acid, maleic acid, lactic acid, citric acid, glutamic acid, saccharinic acid, monomethylsebacic acid, 5-oxo-proline, hexane-1-sulfonic acid, nicotinic acid, 2-, 3- or 4-aminobenzoic acid, 2,4,6-trimethyl-benzoic acid, α-liponic acid, acetylglycine, hippuric acid, phosphoric acid and/or aspartic acid. Citric acid and hydrochloric acid are particularly preferred.

Physiologically acceptable salts with cations or bases are salts of the compound in question—in the form of the anion with at least one, preferably inorganic cation—that are physiologically acceptable—in particular when used in humans and/or mammals. Particular preference is given to the salts of the alkali and alkaline earth metals but also to ammonium salts, but in particular to (mono-) or (di-)sodium, (mono-) or (di-)potassium, magnesium or calcium salts.

The first aspect of the present invention relates to a compound according the general formula (I) for the treatment and/or the prophylaxis of disorders and/or diseases which are mediated, at least in part, by TSPO (translocator protein 18 kDa).

Hence, the compounds according to the present invention are useful for the treatment and/or the prophylaxis of disorders and/or diseases which are mediated, at least in part, by TSPO activity.

Disorders and/or diseases, which are mediated, at least in part, by TSPO activity, include
neurological and neurodegenerative disorders,
psychiatric disorders,
brain tumours,
immunological disorders

and inflammatory diseases.

Neurological and neurodegenerative disorders include Ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia.

Psychiatric disorders include

anxiety disorders, particularly panic disorders, post traumatic stress disorder, panic and adult separation disorder, social anxiety, major depression with adult separation anxiety disorder and bipolar disorder with adult separation anxiety disorder, dysthymia, angina pectoris, suicidality and schizophrenia.

Immunological disorders and inflammatory diseases include

neuroinflammation and arthritis, encephalomyelitis and irritable bowel syndrome.

Another embodiment of the present invention therefore relates to a compound according to general formula (I) for the treatment and/or the prophylaxis of disorders and/or diseases, wherein the disorder and/or the disease is selected from the group consisting of
(i) neurological and neurodegenerative disorders, including
Ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia;
(ii) psychiatric disorders, including
anxiety disorders, particularly panic disorders, post traumatic stress disorder, panic and adult separation disorder, social anxiety, major depression with adult separation anxiety disorder and bipolar disorder with adult separation anxiety disorder, dysthymia, angina pectoris, suicidality and schizophrenia;
(iii) brain tumours;
(iv) immunological disorders and inflammatory diseases, including
neuroinflammation and arthritis, encephalomyelitis and irritable bowel syndrome.

A preferred embodiment of the present invention relates to a compound according to general formula (I) for the treatment and/or the prophylaxis of
(i) neurological and neurodegenerative disorders, including
ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia;
(ii) psychiatric disorders, including
anxiety disorders, particularly panic disorders, post traumatic stress disorder, panic and adult separation disorder, social anxiety, major depression with adult separation anxiety disorder and bipolar disorder with adult separation anxiety disorder, dysthymia, angina pectoris, suicidality and schizophrenia;
(iii) brain tumours;
(iv) immunological disorders and inflammatory diseases, including
neuroinflammation and arthritis, encephalomyelitis and irritable bowel syndrome.

Another preferred embodiment of the present invention relates to a compound according to general formula (I) for the treatment and/or the prophylaxis of neurological and neurodegenerative disorders,
including ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia.

Another preferred embodiment of the present invention relates to a compound according to general formula (I) for the treatment and/or the prophylaxis of psychiatric disorders, including anxiety disorders, particularily panic disorders, post traumatic stress disorder, panic and adult separation disorder, social anxiety, major depression with adult separation anxiety disorder and bipolar disorder with adult separation anxiety disorder, dysthymia, angina pectoris, suicidality and schizophrenia.

Yet another preferred embodiment of the present invention relates to a compound according to general formula (I) for the treatment and/or the prophylaxis of brain tumours.

Yet another preferred embodiment of the present invention relates to a compound according to general formula (I) for the treatment and/or the prophylaxis of immunological disorders and inflammatory diseases, including neuroinflammation and arthritis, encephalomyelitis and irritable bowel syndrome.

One embodiment of the present invention relates to a compound of general formula (I), wherein the compound is selected from a compound according to general formula (1-I).

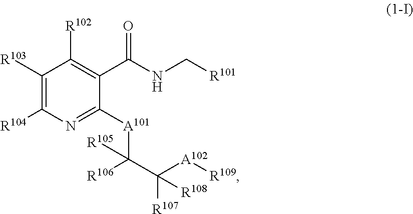

wherein

A¹⁰¹ represents CR¹¹⁰R¹¹¹ or S;

A¹⁰² represents CR¹¹²R¹¹³, C(═O), O, S, S(═O) or S(═O)₂;

R¹⁰¹ represents C₁₋₁₀-alkyl or C₂₋₁₀-heteroalkyl, in each case saturated or unsaturated, branched or unbranched, unsubstituted or mono- or poly-substituted; C₃₋₁₀-cycloalkyl or heterocyclyl, in each case saturated or unsaturated, unsubstituted or mono- or poly-substituted; aryl or heteroaryl, in each case unsubstituted or mono- or poly-substituted; C₁₋₈-alkyl- or C₂₋₈-heteroalkyl-bridged C₃₋₁₀-cycloalkyl or heterocyclyl, in each case saturated or unsaturated, unsubstituted or mono- or poly-substituted, wherein the alkyl or heteroalkyl chain can in each case be branched or unbranched, saturated or unsaturated, unsubstituted or mono- or poly-substituted; or C₁₋₈-alkyl- or C₂₋₈-heteroalkyl-bridged aryl or heteroaryl, in each case unsubstituted or mono- or poly-substituted, wherein the alkyl or heteroalkyl chain can in each case be branched or unbranched, saturated or unsaturated, unsubstituted or mono- or poly-substituted;

R¹⁰², R¹⁰³ and R¹⁰⁴ each independently of the others represents H; F; Cl; Br; I; NO₂; CF₃; CN; OH; OCF₃; SH; SCF₃; methyl; CH₂—O-methyl; CH₂—OH; C₂₋₆-alkyl, O—C₁₋₆-alkyl, S—C₁₋₆-alkyl, in each case saturated or unsaturated, branched or unbranched, unsubstituted or mono- or poly-substituted; C₃₋₇-cycloalkyl, saturated or unsaturated, branched or unbranched, unsubstituted or mono- or poly-substituted; NR^{1a}R^{1b}, wherein R^{1a} and R^{1b} each independently of the other represents H or C₁₋₄-alkyl, saturated or unsaturated, branched or unbranched, unsubstituted or mono- or poly-substituted, or R^{1a} and R^{1b}, together with the nitrogen atom joining them, form a heterocyclyl, saturated or unsaturated, branched or unbranched, unsubstituted or mono- or poly-substituted;

R¹⁰⁵, R¹⁰⁶, R¹⁰⁷, R¹⁰⁸, R¹⁰⁹, R¹¹¹, R¹¹² and R¹¹³ each independently of the others represents H; F; Cl; Br; I; NO₂; CF₃; CN; OH; OCF₃; SH; SCF₃; C₁₋₁₀-alkyl, C₂₋₁₀-heteroalkyl, O—C₁₋₁₀-alkyl or S—C₁₋₁₀-alkyl, in each case saturated or unsaturated, branched or unbranched, unsubstituted or mono- or poly-substituted; C₃₋₁₀-cycloalkyl, saturated or unsaturated, branched or unbranched, unsubstituted or mono- or poly-substituted;

with the proviso that when R¹⁰⁵, R¹⁰⁶, R¹⁰⁷ and R¹⁰⁸ each denotes H and A¹ represents S, A² may not denote S, S(═O) or S(═O)₂;

or R¹⁰⁵ and R¹⁰⁶ or R¹⁰⁷ and R¹⁰⁸ or R¹¹⁰ and R¹¹¹ or R¹¹² and R¹¹³ or R¹⁰⁵ and R¹¹¹ or R¹⁰⁵ and R¹⁰⁷ or R¹⁰⁵ and R¹¹³ or R¹⁰⁷ and R¹¹³ or R¹⁰⁷ and R¹¹¹ or R¹¹¹ and R¹¹³, together with the carbon atom(s) joining them, form a C₃₋₈-cycloalkyl or a heterocyclyl having from three to eight ring members, in each case saturated or unsaturated, unsubstituted or mono- or poly-substituted; wherein the remaining substituents R¹⁰⁵, R¹⁰⁶, R¹⁰⁷, R¹⁰⁸, R¹¹⁰, R¹¹¹, R¹¹² and R¹¹³ in each case have the meaning given above;

R¹⁰⁹ represents C₃₋₁₀-cycloalkyl or heterocyclyl, in each case saturated or unsaturated, unsubstituted or mono- or poly-substituted; aryl or heteroaryl, in each case unsubstituted or mono- or poly-substituted; or represents CR^{1c}R^{1d}, wherein R^{1c} and R^{1d} each independently of the other denotes C₁₋₄-alkyl, saturated or unsaturated, branched or unbranched, unsubstituted or mono- or poly-substituted;

with the proviso that when A¹⁰² represents O or S and R¹⁰⁹ represents heterocyclyl, saturated or unsaturated, unsubstituted or mono- or poly-substituted; or heteroaryl, unsubstituted or mono- or poly-substituted, the bonding of the heteroaryl or heterocyclyl takes place via a carbon atom of the heteroaryl or heterocyclyl;

wherein “alkyl substituted”, “heteroalkyl substituted”, “heterocyclyl substituted” and “cycloalkyl substituted” denote the substitution of one or more hydrogen atoms, in each case independently of one another, by F; Cl; Br; I; NO₂; CF₃; CN; ═O; C₁₋₈-alkyl; C₂₋₈-heteroalkyl; aryl; heteroaryl; C₃₋₁₀-cycloalkyl; heterocyclyl; C₁₋₈-alkyl- or C₂₋₈-heteroalkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂, NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; and S(═O)₂—NH—C₁₋₈-heteroaryl;

wherein “aryl substituted” and “heteroaryl substituted” denote the substitution of one or more hydrogen atoms, in each case independently of one another, by F; Cl; Br; I; NO₂; CF₃; CN; C₁₋₈-alkyl; C₂₋₈-heteroalkyl; aryl; heteroaryl; C₃₋₁₀-cycloalkyl; heterocyclyl; C₁₋₈-alkyl- or C₂₋₈-heteroalkyl-bridged aryl, heteroaryl, C₃₋₁₀-cycloalkyl or heterocyclyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂; NH—C₁₋₈-alkyl; N(C₁₋₈-alkyl)₂; NH—C(═O)C₁₋₈-alkyl; N(C₁₋₈-alkyl)-C(═O)C₁₋₈-alkyl; N(C(═O)C₁₋₈-alkyl)₂; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; S(═O)₂—NH—C₁₋₈-heteroaryl.

The compounds according to general formula (1-I) are known from WO 2010/102809 A1, which is fully included by reference.

In preferred embodiments of the compounds of the general formula (1-I) according to the invention

A¹⁰¹ represents S and

A¹⁰² represents CR¹¹²R¹¹³, O, S or S(═O)₂, preferably CR¹¹²R¹¹³, S or S(═O)₂, particular preferably CR¹¹²R¹¹³.

Further preferred embodiments of the compounds of the general formula (1-I) according to the invention have the general formula (1-I-a), (1-I-b), (1-I-c), (1-I-d), (1-I-e) or (1-I-f):

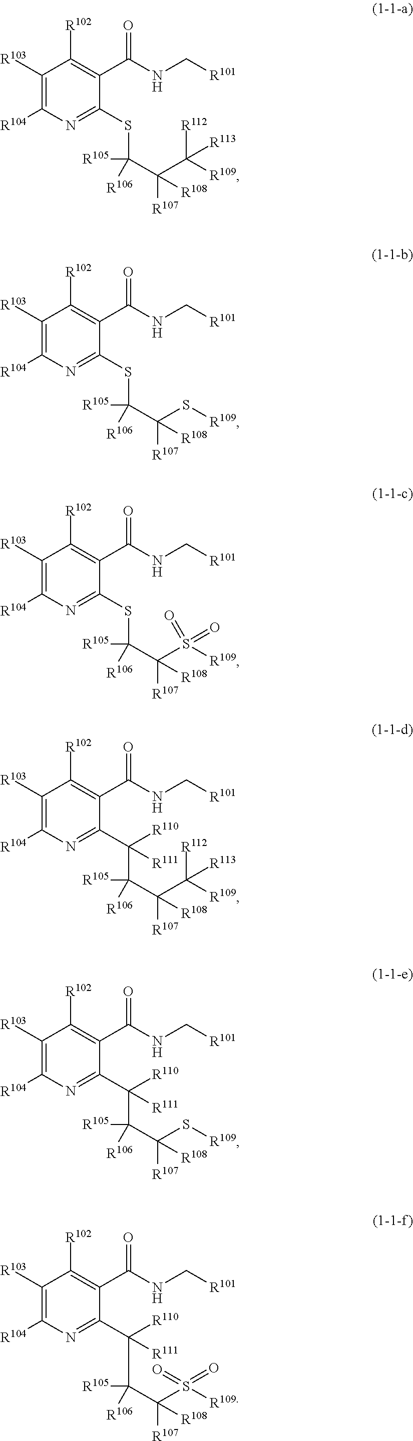

Compounds of the general formulae (1-I-a), (1-I-b) and (1-I-c) are most particularly preferred.

Compounds of the general formula (1-la) are especially preferred.

In a further preferred embodiment the radical R¹⁰¹ represents
C₁₋₁₀-alkyl or C₂₋₁₀-heteroalkyl, in each case saturated or unsaturated, branched or unbranched, unsubstituted or mono- or poly-substituted by one or more substituents each selected independently of one another from the group consisting of F, Cl, Br, I, NO₂, CF₃, CN, OH, ═O, C(═O)—OH, OCF₃, NH₂, S(═O)₂OH, SH, SCF₃, C₁₋₈-alkyl, O—C₁₋₈-alkyl, S—C₁₋₈-alkyl, NH—C₁₋₈-alkyl, N(C₁₋₈-alkyl)₂, C₃₋₁₀-cycloalkyl and heterocyclyl, wherein the above alkyl radicals can in each case be saturated or unsaturated, branched or unbranched, unsubstituted or mono- or poly-substituted by one or more substituents each selected independently of one another from the group consisting of F, Cl, Br, I, O—C₁₋₈-alkyl, OH and OCF₃, and wherein C₃₋₁₀-cycloalkyl or heterocyclyl can in each case be saturated or unsaturated, unsubstituted or mono- or poly-substituted by one or more substituents each selected independently of one another from the group consisting of F, Cl, Br, I, C₁₋₈-alkyl, OH, ═O, O—C₁₋₈-alkyl, OCF₃, NH₂, NH—C₁₋₈-alkyl and N(C₁₋₈-alkyl)₂; or C₃₋₁₀-cycloalkyl or heterocyclyl, in each case saturated or unsaturated, unsubstituted or mono- or poly-substituted by one or more substituents each selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, phenyl, pyridyl and thienyl, wherein benzyl, phenyl, pyridyl, thienyl can in each case be unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃ and S(═O)₂OH;
or aryl or heteroaryl, in each case unsubstituted or mono- or poly-substituted by one or more substituents each selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, phenyl, pyridyl and thienyl, wherein benzyl, phenyl, pyridyl, thienyl can in each case be unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃ and S(═O)₂OH;
or C₁₋₈-alkyl- or C₂₋₈-heteroalkyl-bridged C₃₋₁₀-cycloalkyl or heterocyclyl, in each case saturated or unsaturated, unsubstituted or mono- or poly-substituted by one or more substituents each selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, phenyl, pyridyl and thienyl, wherein benzyl, phenyl, pyridyl, thienyl can in each case be unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃ and S(═O)₂OH;
   wherein the alkyl or heteroalkyl chain can in each case be branched or unbranched, saturated or unsaturated, unsubstituted or mono- or poly-substituted by one or more substituents each selected independently of one another from the group consisting of F, Cl, Br, I, NO₂, CF₃, CN, OH, ═O, C(═O)—OH, OCF₃, NH₂, S(═O)₂OH, SH, SCF₃, C₁₋₈-alkyl, O—C₁₋₈-alkyl, S—C₁₋₈-alkyl, NH—C₁₋₈-alkyl, N(C₁₋₈-alkyl)₂, C₃₋₁₀-cycloalkyl and heterocyclyl, wherein the above alkyl radicals can in each case be saturated or unsaturated, branched or unbranched, unsubstituted or mono- or poly-substituted by one or more substituents each selected independently of one another from the group consisting of F, Cl, Br, I, O—C₁₋₈-alkyl, OH and OCF₃, and wherein C₃₋₁₀-cycloalkyl or heterocyclyl can in each case be saturated or unsaturated, unsubstituted or mono- or poly-substituted by one or more substituents each selected independently of one another from the group consisting of F, Cl, Br, I, C₁₋₈-alkyl, OH, ═O, O—C₁₋₈-alkyl, OCF₃, NH₂, NH—C₁₋₈-alkyl and N(C₁₋₈-alkyl)₂;
or C₁₋₈-alkyl- or C₂₋₈-heteroalkyl-bridged aryl or heteroaryl, in each case unsubstituted or mono- or poly-substituted by one or more substituents each selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, phenyl, pyridyl and thienyl, wherein benzyl, phenyl, pyridyl, thienyl can in each case be unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃ and S(═O)₂OH;
wherein the alkyl or heteroalkyl chain can in each case be branched or unbranched, saturated or unsaturated, unsubstituted or mono- or poly-substituted by one or more substituents each selected independently of one another from the group consisting of F, Cl, Br, I, NO₂, CF₃, CN, OH, ═O, C(═O)—OH, OCF₃, NH₂, S(═O)₂OH, SH, SCF₃, C₁₋₈-alkyl, O—C₁₋₈-alkyl, S—C₁₋₈-alkyl, NH—C₁₋₈-alkyl, N(C₁₋₈-alkyl)₂, C₃₋₁₀-cycloalkyl and heterocyclyl, wherein the above alkyl radicals can in each case be saturated or unsaturated, branched or unbranched, unsubstituted or mono- or poly-substituted by one or more substituents each selected independently of one another from the group consisting of F, Cl, Br, I, O—C₁₋₈-alkyl, OH and OCF₃, and wherein C₃₋₁₀-cycloalkyl or heterocyclyl can in each case be saturated or unsaturated, unsubstituted or mono- or poly-substituted by one or more substituents each selected independently of one another from the group consisting of F, Cl, Br, I, C₁₋₈-alkyl, OH, ═O, O—C₁₋₈-alkyl, OCF₃, NH₂, NH—C₁₋₈-alkyl and N(C₁₋₈-alkyl)₂.

In a further preferred embodiment the substituent R¹⁰¹ represents the following partial structure (1-T1),

—(CR^{114a}R^{114b})ₘ₁—(Y¹)ₙ₁—B¹ (1-T1),

wherein

R^{114a} and R^{114b} each independently of the other represents H; F; Cl; Br; I; NO₂; CF₃; CN; OH; OCF₃; NH₂; C₁₋₄-alkyl, O—C₁₋₄-alkyl, NH—C₁₋₄-alkyl, N(C₁₋₄-alkyl)₂, in each case saturated or unsaturated, branched or unbranched, unsubstituted or mono- or poly-substituted by one or more substituents each selected independently of one another from the group consisting of F, Cl, Br, I, O—C₁₋₄-alkyl, OH and OCF₃; C₃₋₁₀-cycloalkyl or heterocyclyl, in each case saturated or unsaturated, unsubstituted or mono- or poly-substituted by one or more substituents each selected independently of one another from the group consisting of F, Cl, Br, I, C₁₋₄-alkyl, OH, ═O, O—C₁₋₄-alkyl, OCF₃, NH₂, NH—C₁₋₄-alkyl and N(C₁₋₄-alkyl)₂;
m1 represents 0, 1, 2 or 3;
Y¹ represents O or NR¹¹⁵,
   wherein R¹¹⁵ represents H; C₁₋₄-alkyl, saturated or unsaturated, branched or unbranched, unsubstituted or mono- or poly-substituted by one or more substituents each selected independently of one another from the group consisting of F, Cl, Br, I, C₁₋₄-alkyl, OH, O—C₁₋₄-alkyl, OCF₃, NH₂, NH—C₁₋₄-alkyl and N(C₁₋₄-alkyl)₂; or represents C₃₋₁₀-cycloalkyl, saturated or unsaturated, unsubstituted or mono- or poly-substituted by one or more substituents each selected independently of one another from the group consisting of F, Cl, Br, I, C₁₋₄-alkyl, OH, O—C₁₋₄-alkyl, OCF₃, NH₂, NH—C₁₋₄-alkyl and N(C₁₋₄-alkyl)₂;
n1 represents 0 or 1,
B1 represents C₁₋₈-alkyl, saturated or unsaturated, branched or unbranched, unsubstituted or mono- or poly-substituted by one or more substituents each selected independently of one another from the group consisting of F, Cl, Br, I, NO₂, CN, OH, ═O, O—C₁₋₄-alkyl, OCF₃, C(═O)—OH, CF₃, NH₂, NH(C₁₋₄-alkyl), N(C₁₋₄-alkyl)₂, SH, S—C₁₋₄-alkyl, SCF₃ and S(═O)₂OH; C₃₋₁₀-cycloalkyl or heterocyclyl, in each case saturated or unsaturated, unsubstituted or mono- or poly-substituted by one or more substituents each selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, phenyl, pyridyl and thienyl,
   wherein benzyl, phenyl, pyridyl, thienyl can in each case be unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃ and S(═O)₂OH; aryl or heteroaryl, in each case unsubstituted or mono- or poly-substituted by one or more substituents each selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃, S(═O)₂OH, benzyl, phenyl, pyridyl and thienyl, wherein benzyl, phenyl, pyridyl, thienyl can in each case be unsubstituted or mono- or poly-substituted by one or more substituents selected independently of one another from the group consisting of F, Cl, Br, I, NO₂, CN, OH, O—C₁₋₈-alkyl, OCF₃, C₁₋₈-alkyl, C(═O)—OH, CF₃, NH₂, NH(C₁₋₈-alkyl), N(C₁₋₈-alkyl)₂, SH, S—C₁₋₈-alkyl, SCF₃ and S(═O)₂OH.

Preferably,

R^{114a} and R^{114b} each independently of the other represents H; F; Cl; Br; I; NO₂; CF₃; CH₂CF₃; CN; OH; OCF₃, NH₂; C₁₋₄-alkyl, O—C₁₋₄-alkyl, O—C₁₋₄-alkyl-OH, O—C₁₋₄-alkyl-OCH₃, NH—C₁₋₄-alkyl, N(C₁₋₄-alkyl)₂, in each case saturated or unsaturated, branched or unbranched, unsubstituted; C₃₋₁₀-cycloalkyl, saturated or unsaturated, unsubstituted or mono- or poly-substituted by one or more substituents each selected independently of one another from the group consisting of F, Cl, Br, I, C₁₋₄-alkyl, OH, O—C₁₋₄-alkyl;
m1 represents 0, 1, 2 or 3;
Y¹ represents O or NR¹¹⁵;
   wherein R¹¹⁵ represents H; C₁₋₄-alkyl, saturated or unsaturated, unsubstituted; or represents C₃₋₁₀-cycloalkyl, saturated or unsaturated, unsubstituted;
n1 represents 0 or 1;
B¹ represents C₁₋₈-alkyl, saturated or unsaturated, branched or unbranched, unsubstituted or mono- or poly-substituted by one or more substituents each selected independently of one another from the group consisting of F, Cl, Br, I, NO₂, CN, OH, O—C₁₋₄-alkyl, OCF₃, CF₃, NH₂, NH(C₁₋₄-alkyl), N(C₁₋₄-alkyl)₂, SCF₃; C₃₋₁₀-cycloalkyl or heterocyclyl, in each case saturated or unsaturated, unsubstituted or mono- or poly-substituted by one or more substituents each selected independently of one another from the group consisting of F, Cl, Br, I, NO₂, CN, OH, O—C₁₋₄-alkyl, OCF₃, C₁₋₄-alkyl, CF₃, NH₂, NH(C₁₋₄-alkyl), N(C₁₋₄-alkyl)₂, SCF₃; aryl or heteroaryl, in each case unsubstituted or mono- or poly-substituted by one or more substituents each selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃, S(═O)₂OH, benzyl, phenyl and pyridyl, wherein benzyl, phenyl or pyridyl are in each case unsubstituted or mono- or poly-substituted by one or more substituents each selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH.

Particularly preferably, R^{114a} and R^{114b} each independently of the other represents H; F; Cl; Br; I; NO₂; CF₃; CN; methyl; ethyl; n-propyl; isopropyl; cyclopropyl; n-butyl; sec-butyl; tert-butyl; CH₂CF₃; OH; O-methyl; O-ethyl; O—(CH₂)₂—O—CH₃; O—(CH₂)₂—OH; OCF₃; NH₂; NH-methyl; N(methyl)₂; NH-ethyl; N(ethyl)₂; or N(methyl)(ethyl);
m1 represents 0, 1 or 2;
n1 represents 0; and
B¹ represents C₁₋₄-alkyl, saturated, branched or unbranched, unsubstituted or mono- or poly-substituted by one or more substituents each selected independently of one another from the group consisting of F, Cl, Br, I, OH, O—C₁₋₄-alkyl, OCF₃ and CF₃; C₃₋₁₀-cycloalkyl, saturated, unsubstituted; phenyl, naphthyl, pyridyl, thienyl, in each case unsubstituted or mono- or di- or tri-substituted by one, two or three substituents each selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃, S(═O)₂OH.

Most particularly preferably, R^{114a} and R^{114b} each independently of the other represents H; F; Cl; Br; I; methyl; ethyl; n-propyl; isopropyl; n-butyl; sec-butyl; tert-butyl; OH; O-methyl; O-ethyl; O—(CH₂)₂—O—CH₃; or O—(CH₂)₂—OH;
m1 represents 0, 1 or 2;
n1 represents 0; and
B1 represents methyl; ethyl; n-propyl; isopropyl; n-butyl; sec-butyl; tert-butyl; cyclopropyl; cyclobutyl; cyclopentyl; cyclohexyl; cycloheptyl; adamantyl; bicyclo[2.2.1]heptyl; bicyclo[2.2.2]octyl; phenyl, pyridyl, thienyl, in each case unsubstituted or mono-, di- or tri-substituted by one, two or three substituents each selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH.

For n1=0, the partial structure (1-T1) for R¹⁰¹ yields the partial structure (1-T1-1):

—(CR^{114a}R^{114b})ₘ₁—B¹ (1-T1-1).

In a preferred embodiment for n1=0, R^{114a} and R^{114b} each independently of the other represents H; F; Cl; Br; I; methyl; ethyl; n-propyl; isopropyl; n-butyl; sec-butyl; tert-butyl; OH; O-methyl; O-ethyl; O—(CH₂)₂—O—CH₃; or O—(CH₂)₂—OH.

In a preferred embodiment for m1=0, B¹ represents phenyl, pyridyl or thienyl, mono- or di- or tri-substituted by one, two or three substituents each selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃, S(═O)₂OH.

In a preferred embodiment for m1=1 or 2, B¹ represents cyclopropyl; cyclobutyl; cyclopentyl; cyclohexyl; cycloheptyl; adamantyl; bicyclo[2.2.1]heptyl; bicyclo[2.2.2]octyl.

In a preferred embodiment for m1=0, 1 or 2, B¹ represents methyl; ethyl; n-propyl; isopropyl; n-butyl; sec-butyl or tert-butyl.

A further particularly preferred embodiment of the compounds of the general formula (I-1) according to the invention has the general formula (I-1-2):

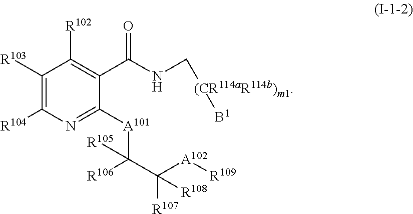

In a further preferred embodiment,
the radicals R¹⁰², R¹⁰³ and R¹⁰⁴ each independently of the others represent H; F; Cl; Br; I; NO₂; CF₃; CN; OH; OCF₃; SH; SCF₃; methyl; CH₂—O-methyl; CH₂—OH; C₂₋₆-alkyl, O—C₁₋₆-alkyl or S—C₁₋₆-alkyl, in each case saturated or unsaturated, branched or unbranched, unsubstituted or mono- or poly-substituted by one or more substituents each selected independently of one another from the group consisting of F, Cl, Br, I, OH, ═O and O—C₁₋₄-alkyl; C₃₋₇-cycloalkyl, saturated or unsaturated, branched or unbranched, unsubstituted; NR^{1a}R^{1b},
   wherein R^{1a} and R^{1b} each independently of the other represents H or C₁₋₄-alkyl, saturated or unsaturated, branched or unbranched, unsubstituted or mono- or poly-substituted by one or more substituents each selected independently of one another from the group consisting of OH, ═O and O—C₁₋₄-alkyl,
   or R^{1a} and R^{1b}, together with the nitrogen atom joining them, form a heterocyclyl, saturated or unsaturated, unsubstituted or mono- or poly-substituted by C₁₋₄-alkyl.

Preferably,
the radicals R¹⁰², R¹⁰³ and R¹⁰⁴ each independently of the others represent H; F; Cl; Br; I; NO₂; CF₃; CN; OH; OCF₃; SH; SCF₃; methyl; ethyl; n-propyl; isopropyl; butyl; sec-butyl; tert-butyl; CH₂CF₃; O-methyl; O-ethyl; O-n-propyl; O-isopropyl; O-butyl; O-sec-butyl; O-tert-butyl; O—(CH₂)₂—O-methyl; O—(CH₂)₂—OH; O—(C═O)-methyl; O—(C═O)-ethyl; S-methyl; S-ethyl; cyclopropyl; cyclobutyl; NR^{1a}R^{1b},
   wherein R^{1a} and R^{1b} each independently of the other is selected from the group consisting of H, methyl, ethyl, (CH₂)₂—O-methyl, (CH₂)₂—OH, (C═O)-methyl, (C═O)-ethyl,
   or R^{1a} and R^{1b}, together with the nitrogen atom joining them, form a pyrrolidinyl, piperidinyl, 4-methylpiperazinyl or morpholinyl.

Particularly preferably,
the radicals R¹⁰², R¹⁰³ and R¹⁰⁴ each independently of the others represent H; F; Cl; Br; I; methyl; ethyl; n-propyl, isopropyl; cyclopropyl; CN; CF₃; O-methyl; OCF₃; S-methyl; SCF₃, pyrrolidinyl, N(methyl)₂.

Most particularly preferably, the radicals R¹⁰², R¹⁰³ and R¹⁰⁴ each independently of the others represent H; F; Cl; methyl; ethyl; O-methyl; CF₃; in particular H.

In a further preferred embodiment, the radicals R¹⁰⁵, R¹⁰⁶, R¹⁰⁷, R¹⁰⁸, R¹¹⁰, R¹¹¹, R¹¹² and R¹¹³ each independently of the others represents H; F; Cl; Br; I; NO₂; CF₃; CN; OH; OCF₃; SH; SCF₃; C₁₋₆-alkyl; O—C₁₋₆-alkyl or S—C₁₋₆-alkyl, in each case saturated or unsaturated, branched or unbranched, unsubstituted or mono- or poly-substituted by one or more substituents each selected independently of one another from the group consisting of F, Cl, Br, I, OH and O—C₁₋₄-alkyl; C₃₋₇-cycloalkyl, saturated or unsaturated, branched or unbranched, unsubstituted or mono- or poly-substituted by one or more substituents each selected independently of one another from the group consisting of F, Cl, Br, I, OH, ═O and O—C₁₋₄-alkyl;

or R¹⁰⁵ and R¹⁰⁶ or R¹⁰⁷ and R¹⁰⁸ or R¹¹⁰ and R¹¹¹ or R¹¹² and R¹¹³ or R¹⁰⁵ and R¹¹¹ or R¹⁰⁵ and R¹⁰⁷ or R¹⁰⁵ and R¹¹³ or R¹⁰⁷ and R¹¹³ or R¹⁰⁷ and R¹¹¹ or R¹¹¹ and R¹¹³, together with the carbon atom(s) joining them, form a cyclopropyl, cyclobutyl, cyclopentyl, cyclohexyl, pyrrolidinyl or piperidinyl, in each case unsubstituted or mono- or poly-substituted by one or more substituents each selected independently of one another from the group consisting of F, Cl, Br, I, OH, ═O and O—C₁₋₄-alkyl; wherein the remaining substituents R¹⁰⁵, R¹⁰⁶, R¹⁰⁷, R¹⁰⁸, R¹¹⁰, R¹¹¹, R¹¹² and R¹¹³ in each case have the meaning given above.

Preferably, the radicals R¹⁰⁵, R¹⁰⁶, R¹⁰⁷, R¹⁰⁸, R¹¹⁰, R¹¹¹, R¹¹² and R¹¹³ each independently of the others represents H; F; Cl; Br; I; NO₂; CF₃; CN; OH; OCF₃; SH; SCF₃; methyl; ethyl; n-propyl; isopropyl; n-butyl; sec-butyl; tert-butyl; O-methyl; O-ethyl; O—(CH₂)₂—O—CH₃; O—(CH₂)₂—OH; S-methyl; S-ethyl; cyclopropyl; cyclobutyl; cyclopentyl; cyclohexyl;

or R¹⁰⁵ and R¹⁰⁶ or R¹⁰⁷ and R¹⁰⁸ or R¹¹⁰ and R¹¹¹ or R¹¹² and R¹¹³ or R¹⁰⁵ and R¹¹¹ or R¹⁰⁵ and R¹⁰⁷ or R¹⁰⁵ and R¹¹³ or R¹⁰⁷ and R¹¹³ or R¹⁰⁷ and R¹¹¹ or R¹¹¹ and R¹¹³, together with the carbon atom(s) joining them, form a cyclopropyl, cyclobutyl, cyclopentyl or cyclohexyl, in each case unsubstituted; wherein the remaining substituents R¹⁰⁵, R¹⁰⁶, R¹⁰⁷, R¹⁰⁸, R¹¹⁰, R¹¹¹, R¹¹² and R¹¹³ in each case have the meaning given above.

Particularly preferably, the radicals R¹⁰⁵, R¹⁰⁶, R¹⁰⁷, R¹⁰⁸, R¹¹⁰, R¹¹¹, R¹¹² and R¹¹³ each independently of the others represents H; F; Cl; Br; I; CN; CF₃; OCF₃; SCF₃; methyl; ethyl; n-propyl, isopropyl; cyclopropyl; O-methyl; S-methyl;

or R¹⁰⁵ and R¹⁰⁷ form a cyclopropyl, cyclobutyl, cyclopentyl or cyclohexyl ring substituted as desired by H, F, Cl, Me, Et, OMe, preferably an unsubstituted cyclopentyl or cyclohexyl ring.

Most particularly preferably, the radicals R¹⁰⁵, R¹⁰⁶, R¹⁰⁷, R¹⁰⁸, R¹¹⁰, R¹¹¹, R¹¹² and R¹¹³ each independently of the others represents F; Cl; H; methyl; ethyl; n-propyl, isopropyl; cyclopropyl; in particular F; H; methyl; or ethyl.

In particular, the radicals R¹¹² and R¹¹³ each independently of the other represents H, F or methyl.

In a further preferred embodiment,
the radical R¹⁰⁹ represents C₃₋₁₀-cycloalkyl or heterocyclyl, in each case saturated or unsaturated, unsubstituted or mono- or poly-substituted by one or more substituents each selected independently of one another from the group consisting of F, Cl, Br, I, NO₂, CN, OH, ═O, O—C₁₋₄-alkyl, OCF₃, C₁₋₄-alkyl, CF₃, SH, S—C₁₋₄-alkyl and SCF₃; aryl or heteroaryl, in each case unsubstituted or mono- or poly-substituted by one or more substituents each selected independently of one another from the group consisting of F, Cl, Br, I, NO₂, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₄-alkyl and SCF₃; or represents CR^{1c}R^{1d},
   wherein R^{1c} and R^{1d} each independently of the other denotes C₁₋₄-alkyl, saturated or unsaturated, branched or unbranched, unsubstituted or mono- or poly-substituted by one or more substituents each selected independently of one another from the group consisting of F, Cl, Br, I, OH, O—C₁₋₄-alkyl, CF₃, OCF₃ and SCF₃.
Preferably, R¹⁰⁹ represents C₃₋₇-cycloalkyl, saturated or unsaturated, pyrrolidinyl, piperazinyl, 4-methylpiperazinyl, piperidinyl, morpholinyl, in each case unsubstituted or mono- or poly-substituted by one or more substituents each selected independently of one another from the group consisting of F, Cl, Br, I, OH, O—C₁₋₄-alkyl, OCF₃, C₁₋₄-alkyl, CF₃, SH, S—C₁₋₄-alkyl and SCF₃; phenyl, naphthyl, pyridyl or thienyl, in each case unsubstituted or mono- or poly-substituted by one or more substituents each selected independently of one another from the group consisting of F, Cl, Br, I, NO₂, CN, OH, O—C₁₋₄-alkyl, OCF₃, C₁₋₄-alkyl, CF₃, SH, S—C₁₋₄-alkyl and SCF₃; or represents CR^{1c}R^{d},
   wherein R^{1c} and R^{1d} each independently of the other denotes C₁₋₄-alkyl, saturated or unsaturated, branched or unbranched, unsubstituted or mono- or poly-substituted by one or more substituents each selected independently of one another from the group consisting of F, Cl, Br, I, OH and O—C₁₋₄-alkyl.

Particularly preferably, the radical R¹⁰⁹ is selected from the group consisting of phenyl, pyridyl or thienyl, in each case unsubstituted or mono- or poly-substituted by one or more substituents each selected independently of one another from the group consisting of F, Cl, Br, I, CN, OH, O—C₁₋₄-alkyl, OCF₃, C₁₋₄-alkyl, CF₃, SH, S—C₁₋₄-alkyl and SCF₃.

Most particularly preferably, R¹⁰⁹ represents phenyl, pyridyl and thienyl, unsubstituted or mono- or poly-substituted by one or more substituents each selected independently of one another from the group consisting of F, Cl, Br, I, CN, methyl, ethyl, n-propyl, isopropyl, n-butyl, sec-butyl, tert-butyl, O-methyl, O-ethyl, O-n-propyl, O-isopropyl, O-butyl, O-sec-butyl, O-tert-butyl, OH, OCF₃, CF₃, SH, S—C₁₋₄-alkyl and SCF₃.

In particular, R¹⁰⁹ represents phenyl, unsubstituted or mono- or poly-substituted by one or more substituents each selected independently of one another from the group consisting of F, Cl, Br, I, methyl, ethyl, n-propyl, isopropyl, n-butyl, sec-butyl, tert-butyl, O-methyl, O-ethyl, OCF₃, CF₃ and SCF₃.

In a further, particularly preferred embodiment

A¹⁰¹ represents S and

A¹⁰² represents CR¹¹²R¹¹³,

R¹⁰¹ represents the partial structure (1-T1-1)

—(CR^{114a}R^{114b})ₘ₁—B¹ (1-T1-1),

wherein

R^{114a} and R^{114b} each independently of the other represents H; F; Cl; Br; I; methyl; ethyl; n-propyl; isopropyl; n-butyl; sec-butyl; tert-butyl; OH; O-methyl; O-ethyl; O—(CH₂)₂—O—CH₃; or O—(CH₂)₂—OH;

m1 represents 0, 1 or 2;

B¹ represents methyl; ethyl; n-propyl; isopropyl; n-butyl; sec-butyl; tert-butyl; cyclopropyl; cyclobutyl; cyclopentyl; cyclohexyl; cycloheptyl; adamantyl; bicyclo[2.2.1]heptyl; bicyclo[2.2.2]octyl; phenyl, pyridyl, thienyl, in each case unsubstituted or mono-, di- or tri-substituted by one, two or three substituents each selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH;

R¹⁰², R¹⁰³ and R¹⁰⁴ each independently of the others represents H; F; Cl; methyl; ethyl; CF₃ or O-methyl;

R¹⁰⁵, R¹⁰⁶, R¹⁰⁷, R¹⁰⁸, R¹¹⁰, R¹¹¹, R¹¹² and R¹¹³ each independently of the others represents H; F; Cl; methyl; ethyl; n-propyl, isopropyl; cyclopropyl;

R¹⁰⁹ represents phenyl, pyridyl or thienyl, unsubstituted or mono- or poly-substituted by one or more substituents each selected independently of one another from the group consisting of F, Cl, Br, I, CN, methyl, ethyl, n-propyl, isopropyl, n-butyl, sec-butyl, tert-butyl, O-methyl, O-ethyl, O-n-propyl, O-isopropyl, O-butyl, O-sec-butyl, O-tert-butyl, OH, OCF₃, CF₃, SH, S—C₁₋₄-alkyl and SCF₃.

Another embodiment of the present invention relates to a compound of general formula (I), wherein the compound is selected from a compound according to general formula (2-I).

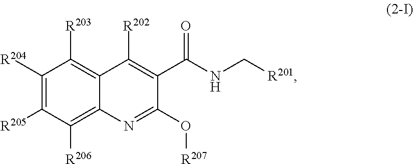

wherein
R²⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R²⁰² represents H; F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R²⁰³, R²⁰⁴, R²⁰⁵ and R²⁰⁶ each independently of one another represent H; F; Cl; Br; I; CN; CF₃; C(═O)H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂—C₁₋₄-aliphatic residue, a N(C₁₋₄ aliphatic residue)-C(═O)—C₁₋₄ aliphatic residue, or a N(C₁₋₄ aliphatic residue)-S(═O)₂—C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R²⁰⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R²⁰⁷ denotes a 3 to 10 membered heterocycloaliphatic residue, the 3 to 10 membered heterocycloaliphatic residue is linked via a carbon atom,

in which an “aliphatic group” and “aliphatic residue” can in each case be branched or unbranched, saturated or unsaturated,

in which a “cycloaliphatic residue” and a “heterocycloaliphatic residue” can in each case be saturated or unsaturated,

in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C_{1- 4}-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

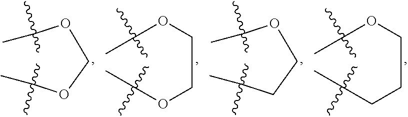 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

The compounds according to general formula (2-I) are known from WO 2012/025236 A1, which is fully included by reference.

Preferred embodiments of the compound according to general formula (2-I) have general formulae (2-Ia), (2-Ib), (2-Ic) or (2-Id):

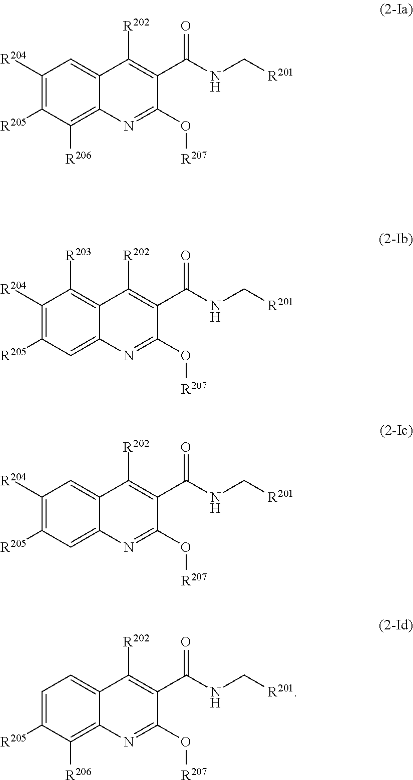

Another preferred embodiment of present invention is a compound according to general formula (2-I), wherein
R²⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

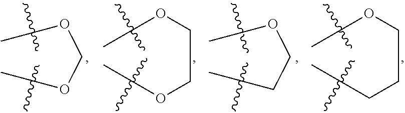 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH,
R²⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄ aliphatic residue may be in each case be unsubstituted or mono- or polysubstituted;
a C₃₋₆-cycloaliphatic residue or a 3 to 6 membered heterocycloaliphatic residue, in each case unsubstituted or mono- or polysubstituted with at least one substituent
   selected from the group consisting of F, Cl, Br, I, ═O, OH, a C₁₋₄-aliphatic residue and an O—C₁₋₄-aliphatic residue,
      wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group,
   which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
R²⁰³, R²⁰⁴, R²⁰⁵ and R²⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, CI, Br, I, ═O, OH, a C₁₋₄-aliphatic residue and a O—C₁₋₄-aliphatic residue, and in each case optionally bridged via an unsubstituted C₁₋₄ aliphatic group,

preferably on the condition that at least one of R²⁰³, R²⁰⁴, R²⁰⁵ and R²⁰⁶ is ≠H,
R²⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
on the condition that if R²⁰⁷ denotes a 3 to 10 membered heterocycloaliphatic residue, the 3 to 10 membered heterocycloaliphatic residue is linked via a carbon atom.

In a preferred embodiment of the compound according to general formula (2-I), the residue
R²⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

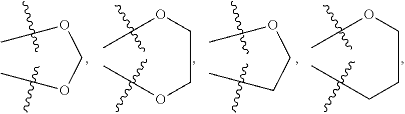 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH.

In a further preferred embodiment of the compound according to general formula (2-I), the residue
R²⁰¹ represents the partial structure (2-T1)

—(CR^{208a}R^{208b})ₘ₂—R^{208c} (2-T1),

wherein
m2 denotes 0, 1, 2, 3 or 4, preferably denotes 0, 1, or 2,
R^{208a} and R^{208b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O,
   preferably each independently of one another represent H, F, Cl, Br, I, NH₂, a NH(C₁₋₄ aliphatic residue), a N(C₁₋₄ aliphatic residue)₂, OH, O—C₁₋₄ aliphatic residue or a C₁₋₄ aliphatic residue,
   more preferably each independently of one another represent H, F, Cl, Br, I, an O—C₁₋₄ aliphatic residue or a C₁₋₄ aliphatic residue,
   even more preferably each independently of one another represent H, F, an O—C₁₋₄ aliphatic residue or a C₁₋₄ aliphatic residue, and
R^{208c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes a C₃₋₁₀-cycloaliphatic residue or a 3 to 10 membered heterocycloaliphatic residue, preferably when m2 is #0, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes—preferably when m2 is =0—an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

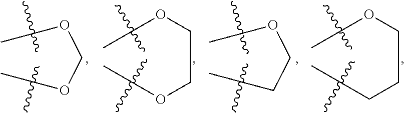 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R²⁰¹ represents the partial structure (2-T1),
   wherein
   m2 denotes 0, 1, or 2,
   R^{208a} and R^{208b} each independently of one another represent H, F, Cl, Br, I, an O—C₁₋₄ aliphatic residue or a C₁₋₄ aliphatic residue,
      preferably each independently of one another represent H, F, a O—C₁₋₂ aliphatic residue or a C₁₋₂ aliphatic residue, and
   R^{208c} denotes a C₁₋₄ aliphatic residue, unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes—preferably when m2 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl or oxazolyl,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein benzyl, phenyl, thienyl, pyridyl, furyl, thiazolyl and oxazolyl may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH.

More preferably,
R²⁰¹ represents the partial structure (2-T1),
   wherein
m2 denotes 0, 1, or 2,
R^{208a} and R^{208b} each independently of one another represent H, F, Cl, Br, I, an O—C₁₋₄ aliphatic residue or a C₁₋₄ aliphatic residue,
preferably each independently of one another represent H, F, a O—C₁₋₂ aliphatic residue or a C₁₋₂ aliphatic residue, and
R^{208c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes—preferably when m2 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl,
   wherein benzyl, phenyl, thienyl and pyridyl, may in each case may be unsubstituted or mono- or polysubstituted, preferably unsubstituted or mono- or disubstituted with at least one substituent selected from the group consisting of F, CI, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH.

In a further preferred embodiment of the compound according to general formula (2-I), the residue
R²⁰¹ represents the partial structure (2-T1),
   wherein
m2 is 0, 1 or 2 and
R^{208a} and R^{208b} each independently of one another represent H, F, a O—C₁₋₄ aliphatic residue or a C₁₋₄ aliphatic residue; preferably H, F, CH₃ or OCH₃;
R^{208c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
   wherein
m2 is 0,
R^{208a} and R^{208b} each independently of one another represent H, F, a O—C₁₋₄ aliphatic residue or a C₁₋₄ aliphatic residue; preferably H, F, CH₃ or OCH₃; and
R^{208c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃.

Particularly preferred is a compound according to general formula (2-I) which has the following general formula (2-Ie):

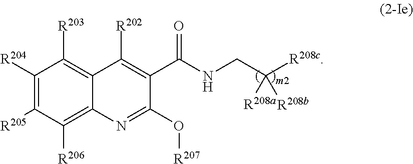

In a preferred embodiment of the compound according to general formula (2-I), the residue
R²⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted.

Preferably,
R²⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

More preferably,
R²⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
cyclopropyl, cyclobutyl, cyclopentyl, cyclohexyl, pyrrolidinyl, piperazinyl, 4-methylpiperazinyl, morpholinyl, or piperidinyl, preferably cyclopropyl, cyclobutyl, cyclopentyl or cyclohexyl, in each case unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
   and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted O—C₁₋₄-aliphatic residue.

Even more preferably,
R²⁰² represents H; F; Cl; Br; I; CN; CF₃; NO₂; OCF₃; SCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; S-Ethyl; cyclopropyl, cyclobutyl, cyclopentyl, and cyclohexyl.

Still more preferably,
R²⁰² is selected from the group consisting of H; F; Cl; CF₃; CN; SCF₃; OCF₃; CH₃; C₂H₅; n-propyl; iso-propyl; t-butyl; cyclopropyl; O—CH₃ and O—C₂H₅;

In particular,
R²⁰² is selected from the group consisting of H; F; Cl; CF₃; CH₃; C₂H₅, iso-propyl; cyclopropyl; and O—CH₃;

In a particular preferred embodiment of the compound according to general formula (2-I), the residue

R²⁰² is ≠H.

In a preferred embodiment of the compound according to general formula (2-I), the residues
R²⁰³, R²⁰⁴, R²⁰⁵ and R²⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group,

preferably on the condition that at least one of R²⁰³, R²⁰⁴, R²⁰⁵ and R²⁰⁶ is ≠H.

Preferably,
R²⁰³, R²⁰⁴, R²⁰⁵ and R²⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a O—C(═O)—C₁₋₄-aliphatic residue, a S—C₁₋₄-aliphatic residue, a S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, preferably on the condition that at least one of R²⁰³, R²⁰⁴, R²⁰⁵ and R²⁰⁶ is ≠H.

More preferably,
R²⁰³, R²⁰⁴, R²⁰⁵ and R²⁰⁶ each independently of one another represent H; F; Cl; Br; I; CN; CF₃; OCF₃; SCF₃; C(═O)H; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a S—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue, unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group,

preferably on the condition that at least one of R²⁰³, R²⁰⁴, R²⁰⁵ and R²⁰⁶ is ≠H.

In a further preferred embodiment of the present invention
R²⁰³, R²⁰⁴, R²⁰⁵ and R²⁰⁶ each independently of one another are selected from the group consisting of H; F; Cl; Br; I; NO₂; CF₃; CN; OCF₃; SCF₃; a (C═O)—C₁₋₄ aliphatic residue, a C₁₋₄ aliphatic residue, O—C₁₋₄ aliphatic residue, a S—C₁₋₄ aliphatic residue, wherein the C₁₋₄-aliphatic residue in each case may be unsubstituted or mono- or polysubstituted with at least one substituent selected from the group consisting of F, Cl, and O—CH₃;

preferably on the condition that at least one of R²⁰³, R²⁰⁴, R²⁰⁵ and R²⁰⁶ is ≠H.

Preferably,
R²⁰³, R²⁰⁴, R²⁰⁵ and R²⁰⁶ each independently of one another are selected from the group consisting of H; F; Cl; Br; I; NO₂; CF₃; CN; OCF₃; SCF₃; methyl; ethyl; n-propyl; iso-propyl; n-butyl; sec.-butyl; tert.-butyl; cyclopropyl; C(═O)-methyl; C(═O)-ethyl; (C═O)-isopropyl; (C═O)-t-butyl; O-methyl; O-ethyl; O-isopropyl; O-t-butyl; O—(CH₂)₂—O—CH₃; S-Methyl; S-Ethyl;

preferably on the condition that at least one of R²⁰³, R²⁰⁴, R²⁰⁵ and R²⁰⁶ is ≠H.

In particular,
R²⁰³, R²⁰⁴, R²⁰⁵ and R²⁰⁶ are each independently of one another are selected from the group consisting of H; F; Cl; Br; I; NO₂; CF₃; CN; (C═O)-methyl; (C═O)-ethyl; (C═O)-isopropyl; (C═O)-t-butyl; methyl; ethyl; isopropyl; t-butyl; O-methyl; O-Ethyl; O-isopropyl; O-t-butyl; OCF₃; S-methyl; S-ethyl; and SCF₃;

preferably on the condition that at least one of R²⁰³, R²⁰⁴, R²⁰⁵ and R²⁰⁶ is ≠H.

More particularly,
R²⁰³, R²⁰⁴, R²⁰⁵ and R²⁰⁶ are each independently of one another selected from the group consisting of H; F; Cl; Br; CF₃; CN; OCF₃ and NO₂;

preferably on the condition that at least one of R²⁰³, R²⁰⁴, R²⁰⁵ and R²⁰⁶ is ≠H.

Most preferred,
R²⁰³, R²⁰⁴ and R²⁰⁶ each independently of one another are selected from the group consisting of H and F; and
R²⁰⁵ denotes F; Br; CF₃; OCF₃; CN; or NO₂.

In a particular preferred embodiment of the compound according to general formula (2-I) at least one of the residues R²⁰³, R²⁰⁴, R²⁰⁵ and R²⁰⁶ is ≠H.

In a preferred embodiment of the compound according to general formula (2-I), the residue
R²⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R²⁰⁷ denotes a 3 to 10 membered heterocycloaliphatic residue, the 3 to 10 membered heterocycloaliphatic residue is linked via a carbon atom.

In a further preferred embodiment of the compound according to general formula (2-I), the residue
R²⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue.
on the condition that if R²⁰⁷ denotes a 3 to 10 membered heterocycloaliphatic residue, the 3 to 10 membered heterocycloaliphatic residue is linked via a carbon atom.

Preferably,
R²⁰⁷ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue.
on the condition that if R²⁰⁷ denotes a 3 to 10 membered heterocycloaliphatic residue, the 3 to 10 membered heterocycloaliphatic residue is linked via a carbon atom.

More preferably,
R²⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, CF₃, a C(═O)—O—C₁₋₄-aliphatic residue, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue is bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue.

Even more preferably,
R²⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue is in each case bridged via a unsubstituted C₁₋₈ aliphatic group, preferably an unsubstituted C₁₋₄ aliphatic group.

Still more preferably,
R²⁰⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue,
   wherein the C₁₋₄-aliphatic residue in each case is unsubstituted,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with OH or an unsubstituted O—C₁₋₄-aliphatic residue,
and wherein the C₃₋₁₀-cycloaliphatic residue or the 3 to 10 membered heterocycloaliphatic residue is in each case bridged via a unsubstituted C₁₋₄ aliphatic group.

In particular,
R²⁰⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue,
   wherein the C₁₋₄-aliphatic residue in each case is unsubstituted.

Most preferred,
R²⁰⁷ denotes a C₁₋₆-aliphatic residue, unsubstituted or mono- or polysubstituted with at least one substituent selected from the group consisting of F, CF₃, Cl, OH, and O-methyl.

Preferred is also a compound according to general formula (2-I), wherein
R²⁰¹ represents the partial structure (2-T1),

—(CR^{208a}R^{208b})ₘ₂—R^{208c} (2-T1),
wherein
m2 is 0, 1 or 2 and
R^{208a} and R^{208b} each independently of one another represent H, F, a O—C₁₋₄ aliphatic residue or a C₁₋₄ aliphatic residue; preferably H, F, CH₃ or OCH₃;
R^{208c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
wherein
m2 is 0,
R^{208a} and R^{208b} each independently of one another represent H, F, a O—C₁₋₄ aliphatic residue or a C₁₋₄ aliphatic residue; preferably H, F, CH₃ or OCH₃; and
R^{208c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
R²⁰² is selected from the group consisting of H; F; Cl; CF₃; CH₃; C₂H₅, iso-propyl; cyclopropyl; and O—CH₃;
R²⁰³, R²⁰⁴, R²⁰⁵ and R²⁰⁶ are each independently of one another selected from the group consisting of H; F; Cl; Br; CF₃; CN; OCF₃ and NO₂;

preferably on the condition that at least one of R²⁰³, R²⁰⁴, R²⁰⁵ and R²⁰⁶ is ≠H,
R²⁰⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue
   wherein the C₁₋₄-aliphatic residue in each case is unsubstituted.

Particularly preferred is also a compound according to general formula (2-I), wherein
R²⁰¹ represents aryl, preferably phenyl, or heteroaryl, preferably pyridyl or thienyl, in each case unsubstituted or mono- or disubstituted with at least one substituent selected from the group consisting of F, Cl, Br, I, OH, OCH₃, OCF₃, CF₃, CN, and CH₃, preferably represents phenyl, unsubstituted or mono- or disubstituted with at least one substituent selected from the group consisting of F, Cl, Br, I, OH, OCH₃, OCF₃, CF₃, CN, and CH₃;
R²⁰² is selected from the group consisting of H, F, Cl, CF₃, CH₃, C₂H₅, iso-propyl, cyclopropyl, and O—CH₃; preferably is selected from the group consisting of CH₃, C₂H₅, OCH₃ and CF₃;
R²⁰³, R²⁰⁴, R²⁰⁵ and R²⁰⁶ are each independently of one another selected from the group consisting of H, F, Cl, Br, CF₃, CN, OCF₃ and NO₂;
preferably on the condition that at least one of R²⁰³, R²⁰⁴, R²⁰⁵ and R²⁰⁶ is ≠H, more preferably on the condition that R²⁰⁵ is ≠H;
R²⁰⁷ denotes a saturated C₁₋₄-aliphatic residue, unsubstituted or mono- or disubstituted with at least one substituent selected from the group consisting of F, Cl, Br, I, OH, O—CH₃, OCF₃, SCF₃, and CF₃.

Another embodiment of the present invention therefore relates to a compound of general formula (I), wherein the compound is selected from a compound according to general formula (3-I),

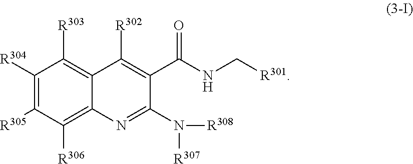

wherein
R³⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R³⁰² represents H; F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R³⁰³, R³⁰⁴, R³⁰⁵ and R³⁰⁶ each independently of one another represent H; F; Cl; Br; I; CN; CF₃; C(═O)H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂—C₁₋₄-aliphatic residue, a N(C₁₋₄ aliphatic residue)-C(═O)—C₁₋₄ aliphatic residue, or a N(C₁₋₄ aliphatic residue)-S(═O)₂—C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
on the condition that at least one of R³⁰³, R³⁰⁴, R³⁰⁵ and R³⁰⁶ is ≠H,
R³⁰⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R³⁰⁷ denotes a 3 to 10 membered heterocycloaliphatic residue, the binding is carried out via a carbon atom of the 3 to 10 membered heterocycloaliphatic residue;
R³⁰⁸ denotes H or a C₁₋₁₀-aliphatic residue, preferably a C₁₋₄-aliphatic residue, unsubstituted or mono- or polysubstituted; or
R³⁰⁷ and R³⁰⁸ form together with the nitrogen atom connecting them a 3 to 10 membered heterocycloaliphatic residue, preferably a 3 to 6 membered heterocycloaliphatic residue, unsubstituted or mono- or polysubstituted;

in which an “aliphatic group” and “aliphatic residue” can in each case be branched or unbranched, saturated or unsaturated,

in which a “cycloaliphatic residue” and a “heterocycloaliphatic residue” can in each case be saturated or unsaturated,

in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C_{1- 4}-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

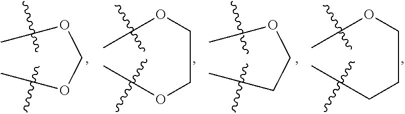 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

The compounds according to general formula (3-I) are known from WO 2012/025237 A1, which is fully included by reference.

Preferred embodiments of the compound according to general formula (3-I) have general formulae (3-Ia), (3-Ib), (3-Ic) or (3-Id):

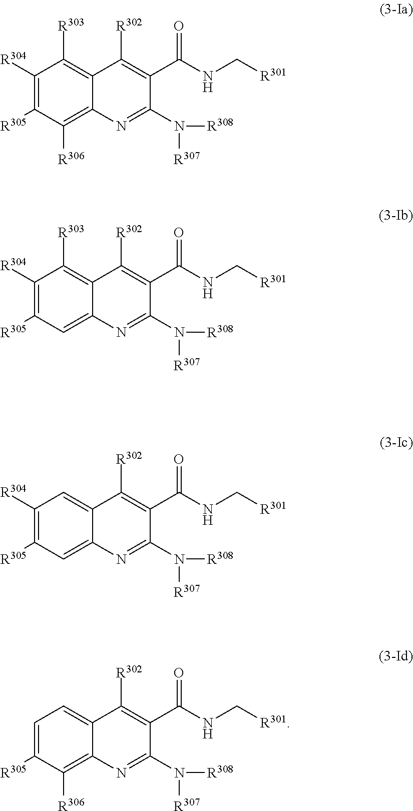

Another preferred embodiment of present invention is a compound according to general formula (I), wherein
R³⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

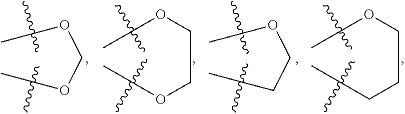 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH,
R³⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, preferably represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and an O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
R³⁰³, R³⁰⁴, R³⁰⁵ and R³⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R³⁰³, R³⁰⁴, R³⁰⁵ and R³⁰⁶ is ≠H,
R³⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R³⁰⁷ denotes a 3 to 10 membered heterocycloaliphatic residue, the 3 to 10 membered heterocycloaliphatic residue is linked via a carbon atom, and
R³⁰⁸ denotes H or a C₁₋₁₀-aliphatic residue, preferably a C₁₋₄-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or
R³⁰⁷ and R³⁰⁸ form together with the nitrogen atom connecting them a 3 to 10 membered heterocycloaliphatic residue, preferably a 3 to 6 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In a preferred embodiment of the compound according to general formula (3-I), the residue
R³⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

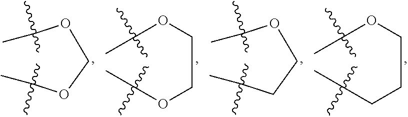 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or poly-substituted with at least one substituent selected from the group consisting of F, CI, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH.

In a further preferred embodiment of the compound according to general formula (I), the residue
R³⁰¹ represents the partial structure (3-T1)

—(CR^{308a}R^{308b})ₘ₃—R^{308c} (3-T1),

wherein
m3 denotes 0, 1, 2, 3 or 4, preferably denotes 0, 1, or 2,
R^{308a} and R^{308b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, preferably each independently of one another represent H, F, Cl, Br, I, NH₂, a NH(C₁₋₄ aliphatic residue), a N(C₁₋₄ aliphatic residue)₂, OH, O—C₁₋₄ aliphatic residue or a C₁₋₄ aliphatic residue,
more preferably each independently of one another represent H, F, Cl, Br, I, an O—C₁₋₄ aliphatic residue or a C₁₋₄ aliphatic residue,
even more preferably each independently of one another represent H, F, an O—C₁₋₄ aliphatic residue or a C₁₋₄ aliphatic residue, and
R^{308c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes a C₃₋₁₀-cycloaliphatic residue or a 3 to 10 membered heterocycloaliphatic residue, preferably when m is ≠0, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes—preferably when m3 is =0—an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

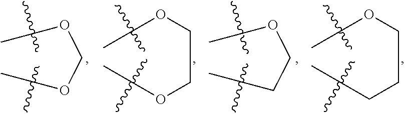 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R³⁰¹ represents the partial structure (3-T1),
   wherein
   m3 denotes 0, 1, or 2,
   R^{308a} and R^{308b} each independently of one another represent H, F, Cl, Br, I, an O—C₁₋₄ aliphatic residue or a C₁₋₄ aliphatic residue,
   preferably each independently of one another represent H, F, a O—C₁₋₂ aliphatic residue or a C₁₋₂ aliphatic residue, and
   R^{308c} denotes a C₁₋₄ aliphatic residue, unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes—preferably when m3 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl or oxazolyl,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein benzyl, phenyl, thienyl, pyridyl, furyl, thiazolyl and oxazolyl may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

More preferably,
R³⁰¹ represents the partial structure (3-T1),
   wherein
   m3 denotes 0, 1, or 2,
   R^{308a} and R^{308b} each independently of one another represent H, F, Cl, Br, I, an O—C₁₋₄ aliphatic residue or a C₁₋₄ aliphatic residue,
   preferably each independently of one another represent H, F, a O—C₁₋₂ aliphatic residue or a C₁₋₂ aliphatic residue, and
   R^{308c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
   or denotes—preferably when m3 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl,
      wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In a further preferred embodiment of the compound according to general formula (3-I), the residue
R³⁰¹ represents the partial structure (3-T1),
   wherein
   m3 is 0, 1 or 2 and
   R^{308a} and R^{308b} each independently of one another represent H, F, a O—C₁₋₄ aliphatic residue or a C₁₋₄ aliphatic residue; preferably H, F, CH₃ or OCH₃;
   R^{308c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
   wherein
   m3 is 0,
   R^{308a} and R^{308b} each independently of one another represent H, F, a O—C₁₋₄ aliphatic residue or a C₁₋₄ aliphatic residue; preferably H, F, CH₃ or OCH₃; and
   R^{308c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
      wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃.

Particularly preferred is a compound according to general formula (3-I) which has the following general formula (3-Ie):

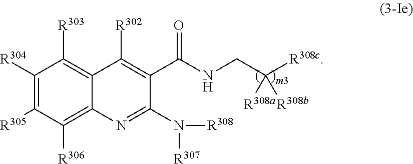

In a preferred embodiment of the compound according to general formula (3-I), the residue
R³⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted.

Preferably,
R³⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

More preferably,
R³⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
cyclopropyl, cyclobutyl, cyclopentyl, cyclohexyl, pyrrolidinyl, piperazinyl, 4-methylpiperazinyl, morpholinyl, or piperidinyl, preferably cyclopropyl, cyclobutyl, cyclopentyl or cyclohexyl, in each case unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue, and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted O—C₁₋₄-aliphatic residue.

Even more preferably,
R³⁰² represents H; F; Cl; Br; I; CN; CF₃; NO₂; OCF₃; SCF₃; methyl; ethyl; n-propyl; iso-propyl; n-butyl; sec-butyl; tert-butyl; O-methyl; O-ethyl; O—(CH₂)₂—O—CH₃; O—(CH₂)₂—OH; S-Methyl; S-Ethyl; cyclopropyl, cyclobutyl, cyclopentyl, and cyclohexyl.

Still more preferably,
R³⁰² is selected from the group consisting of H; F; Cl; Br; CF₃; CN; SCF₃; OCF₃; CH₃; C₂H₅; n-propyl; iso-propyl; t-butyl; cyclopropyl; O—CH₃ and O—C₂H₅.

In particular,
R³⁰² is selected from the group consisting of H; F; Cl; Br; CF₃; CH₃; C₂H₅, iso-propyl; cyclopropyl; and O—CH₃.

In a particular preferred embodiment of the compound according to general formula (3-I), the residue
R³⁰² is ≠H.

In another preferred embodiment of the compound according to general formula (3-I), the residues
R³⁰³, R³⁰⁴, R³⁰⁵ and R³⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R³⁰³, R³⁰⁴, R³⁰⁵ and R³⁰⁶ is ≠H.

Preferably,
R³⁰³, R³⁰⁴, R³⁰⁵ and R³⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a O—C(═O)—C₁₋₄-aliphatic residue, a S—C₁₋₄-aliphatic residue, a S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R³⁰³, R³⁰⁴, R³⁰⁵ and R³⁰⁶ is ≠H.

More preferably,
R³⁰³, R³⁰⁴, R³⁰⁵ and R³⁰⁶ each independently of one another represent H; F; Cl; Br; I; CN; CF₃; OCF₃; SCF₃; C(═O)H; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a S—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue, unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group,
on the condition that at least one of R³⁰³, R³⁰⁴, R³⁰⁵ and R³⁰⁶ is ≠H.

In a further preferred embodiment of the present invention
R³⁰³, R³⁰⁴, R³⁰⁵ and R³⁰⁶ each independently of one another are selected from the group consisting of H; F; Cl; Br; I; NO₂; CF₃; CN; OCF₃; SCF₃; a (C═O)—C₁₋₄aliphatic residue, a C₁₋₄aliphatic residue, O—C₁₋₄ aliphatic residue, a S—C₁₋₄aliphatic residue, wherein the C₁₋₄-aliphatic residue in each case may be unsubstituted or mono- or polysubstituted with at least one substituent selected from the group consisting of F, Cl, and O—CH₃;
on the condition that at least one of R³⁰³, R³⁰⁴, R³⁰⁵ and R³⁰⁶ is ≠H.

Preferably,
R³⁰³, R³⁰⁴, R³⁰⁵ and R³⁰⁶ each independently of one another are selected from the group consisting of H; F; Cl; Br; I; NO₂; CF₃; CN; OCF₃; SCF₃; methyl; ethyl; n-propyl; iso-propyl; n-butyl; sec.-butyl; tert.-butyl; cyclopropyl; C(═O)-methyl; C(═O)-ethyl; (C═O)-isopropyl; (C═O)-t-butyl; O-methyl; O-ethyl; O-isopropyl; O-t-butyl; O—(CH₂)₂—O—CH₃; S-Methyl; S-Ethyl;
on the condition that at least one of R³⁰³, R³⁰⁴, R³⁰⁵ and R³⁰⁶ is ≠H.

In particular,
R³⁰³, R³⁰⁴, R³⁰⁵ and R³⁰⁶ are each independently of one another are selected from the group consisting of H; F; Cl; Br; I; NO₂; CF₃; CN; (C═O)-methyl; (C═O)-ethyl; (C═O)-isopropyl; (C═O)-t-butyl; methyl; ethyl; isopropyl; t-butyl; O-methyl; O-Ethyl; O-isopropyl; O-t-butyl; OCF₃; S-methyl; S-ethyl; and SCF₃;
on the condition that at least one of R³⁰³, R³⁰⁴, R³⁰⁵ and R³⁰⁶ is ≠H.

More particularly,
R³⁰³, R³⁰⁴, R³⁰⁵ and R³⁰⁶ are each independently of one another selected from the group consisting of H; F; Cl; Br; CF₃; CN; OCF₃ and NO₂;
on the condition that at least one of R³⁰³, R³⁰⁴, R³⁰⁵ and R³⁰⁶ is ≠H.

Most preferred,
R³⁰³, R³⁰⁴ and R³⁰⁶ each independently of one another represent H or F; and
R³⁰⁵ denotes F; CF₃; OCF₃; CN; or NO₂.

In a preferred embodiment of the compound according to general formula (3-I), the residue
R³⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O) OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R³⁰⁷ denotes a 3 to 10 membered heterocycloaliphatic residue, the 3 to 10 membered heterocycloaliphatic residue is linked via a carbon atom. and
R³⁰⁸ denotes H or a C₁₋₁₀-aliphatic residue, preferably a C₁₋₆-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or
R³⁰⁷ and R³⁰⁸ form together with the nitrogen atom connecting them a 3 to 10 membered heterocycloaliphatic residue, preferably a 3 to 6 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In a further preferred embodiment of the compound according to general formula (3-I), the residue
R³⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue, on the condition that if R³⁰⁷ denotes a 3 to 10 membered heterocycloaliphatic residue, the 3 to 10 membered heterocycloaliphatic residue is linked via a carbon atom, and
R³⁰⁸ denotes H or a C₁₋₁₀-aliphatic residue, preferably a C₁₋₆-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or
R³⁰⁷ and R³⁰⁸ form together with the nitrogen atom connecting them a 3 to 10 membered heterocycloaliphatic residue, preferably a 3 to 6 membered heterocycloaliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, a C₁₋₄-aliphatic residue, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue.

Preferably,
R³⁰⁷ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
on the condition that if R³⁰⁷ denotes a 3 to 10 membered heterocycloaliphatic residue, the 3 to 10 membered heterocycloaliphatic residue is linked via a carbon atom, and
R³⁰⁸ denotes H or a C₁₋₁₀-aliphatic residue, preferably a C₁₋₆-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or
R³⁰⁷ and R³⁰⁸ form together with the nitrogen atom connecting them a 3 to 10 membered heterocycloaliphatic residue, preferably a 3 to 6 membered heterocycloaliphatic residue, more preferably selected from the group consisting of morpholinyl, piperidinyl, pyrrolidinyl, azetidinyl and piperazinyl, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue.

More preferably,
R³⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, CF₃, a C(═O)—O—C₁₋₄-aliphatic residue, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue is in each case bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue, and
R³⁰⁸ denotes H or a C₁₋₆-aliphatic residue, preferably a C₁₋₄-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or
R³⁰⁷ and R³⁰⁸ form together with the nitrogen atom connecting them a 3 to 6 membered heterocycloaliphatic residue, preferably selected from the group consisting of morpholinyl, piperidinyl, pyrrolidinyl, azetidinyl and piperazinyl, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue.

Even more preferably,
R³⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue is in each case bridged via a unsubstituted C₁₋₈ aliphatic group, preferably an unsubstituted C₁₋₄ aliphatic group, and
R³⁰⁸ denotes H or an unsubstituted C₁₋₄-aliphatic residue, preferably selected from the group consisting of methyl, ethyl, n-propyl, 2-propyl, n-butyl, isobutyl, sec.-butyl and tert.-butyl, more preferably selected from the group consisting of methyl and ethyl or
R³⁰⁷ and R³⁰⁸ form together with the nitrogen atom connecting them a 3 to 6 membered heterocycloaliphatic residue, preferably selected from the group consisting of morpholinyl, piperidinyl, pyrrolidinyl, and azetidinyl, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue.

Still more preferably,
R³⁰⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue,
   wherein the C₁₋₄-aliphatic residue in each case is unsubstituted,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with OH or an unsubstituted O—C₁₋₄-aliphatic residue.
and wherein the C₃₋₁₀-cycloaliphatic residue or the 3 to 10 membered heterocycloaliphatic residue is in each case bridged via a unsubstituted C₁₋₄ aliphatic group,
R³⁰⁸ denotes H or an unsubstituted C₁₋₄-aliphatic residue, preferably H, methyl, ethyl, n-propyl, 2-propyl, n-butyl, isobutyl, sec.-butyl or tert.-butyl, or
R³⁰⁷ and R³⁰⁸ form together with the nitrogen atom connecting them a morpholinyl, piperidinyl, pyrrolidinyl, or azetidinyl, in each case unsubstituted or mono- or polysubstituted with at least one substituent selected from the group consisting of F, Cl, Br, I, OH, an O—C₁₋₄ aliphatic residue, and a C₁₋₄-aliphatic residue,
   wherein the C₁₋₄-aliphatic residue in each case may be unsubstituted or mono- or polysubstituted with OH or an unsubstituted O—C₁₋₄-aliphatic residue.

Most preferred,
R³⁰⁷ denotes a C₁₋₆-aliphatic residue, unsubstituted or mono- or polysubstituted with at least one substituent selected from the group consisting of F, Cl, OH, and O-methyl, preferably unsubstituted or mono- or polysubstituted with at least one substituent selected from the group consisting of F, Cl, and O-methyl, more preferably unsubstituted or mono- or polysubstituted with at least one substituent selected from the group consisting of F and O-methyl,
R³⁰⁸ denotes H, methyl, ethyl, n-propyl, 2-propyl, n-butyl, isobutyl, sec.-butyl or tert.-butyl, preferably methyl or ethyl, or
R³⁰⁷ and R³⁰⁸ form together with the nitrogen atom connecting them a morpholinyl, piperidinyl, pyrrolidinyl, or azetidinyl, in each case unsubstituted or mono- or polysubstituted with at least one substituent selected from the group consisting of F, Cl, Br, I, OH, an O—C₁₋₄ aliphatic residue, and a C₁₋₄-aliphatic residue, more preferably unsubstituted or mono- or polysubstituted with at least one substituent selected from the group consisting of F, Cl and a O—C₁₋₄ aliphatic residue, preferably O-methyl.

Particularly preferred is also a compound according to general formula (I), wherein
R³⁰¹ represents the partial structure (3-T1),

—(CR^{308a}R^{308b})ₘ₃—R^{308c} (3-T1),
wherein
m3 is 0, 1 or 2 and
R^{308a} and R^{308b} each independently of one another represent H, F, a O—C₁₋₄ aliphatic residue or a C₁₋₄ aliphatic residue; preferably H, F, CH₃ or OCH₃;
R^{308c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
wherein
m3 is 0,
R^{308a} and R^{308b} each independently of one another represent H, F, a O—C₁₋₄ aliphatic residue or a C₁₋₄ aliphatic residue; preferably H, F, CH₃ or OCH₃; and
R^{308c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
R³⁰² is selected from the group consisting of H; F; Cl; CF₃; CH₃; C₂H₅, iso-propyl; cyclopropyl; and O—CH₃,
R³⁰³, R³⁰⁴, R³⁰⁵ and R³⁰⁶ are each independently of one another selected from the group consisting of H; F; Cl; CF₃; CN; OCF₃ and NO₂;
on the condition that at least one of R³⁰³, R³⁰⁴, R³⁰⁵ and R³⁰⁶ is ≠H,
R³⁰⁷ denotes a C₁₋₆-aliphatic residue, unsubstituted or mono- or polysubstituted with at least one substituent selected from the group consisting of F, Cl, OH, and O-methyl, preferably unsubstituted or mono- or polysubstituted with at least one substituent selected from the group consisting of F, Cl, and O-methyl, more preferably unsubstituted or mono- or polysubstituted with at least one substituent selected from the group consisting of F and O-methyl,
R³⁰⁸ denotes H, methyl, ethyl, n-propyl, 2-propyl, n-butyl, isobutyl, sec.-butyl or tert.-butyl, preferably methyl or ethyl, or
R³⁰⁷ and R³⁰⁸ form together with the nitrogen atom connecting them a morpholinyl, piperidinyl, pyrrolidinyl, or azetidinyl, in each case unsubstituted or mono- or polysubstituted with at least one substituent selected from the group consisting of F, Cl, Br, I, OH, an O—C₁₋₄ aliphatic residue, and a C₁₋₄-aliphatic residue, more preferably unsubstituted or mono- or polysubstituted with at least one substituent selected from the group consisting of F, CI and a O—C₁₋₄ aliphatic residue, preferably O-methyl.

Another embodiment of the present invention therefore relates to a compound of general formula (I), wherein the compound is selected from a compound according to general formula (4-I),

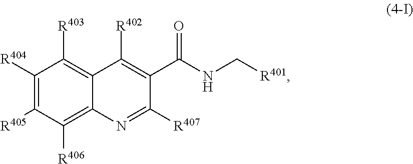

wherein
R⁴⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted,
R⁴⁰² represents H; F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted,
R⁴⁰³, R⁴⁰⁴, R⁴⁰⁵ and R⁴⁰⁶ each independently of one another represent H; F; Cl; Br; I; CN; CF₃; C(═O)H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂—C₁₋₄-aliphatic residue, a N(C₁₋₄ aliphatic residue)-C(═O)—C₁₋₄ aliphatic residue, or a N(C₁₋₄ aliphatic residue)-S(═O)₂—C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
on the condition that at least one of R⁴⁰³, R⁴⁰⁴, R⁴⁰⁵ and R⁴⁰⁶ is ≠H,
R⁴⁰⁷ represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
on the condition that if R⁴⁰⁷ denotes a 3 to 10 membered heterocycloaliphatic residue, the binding is carried out via a carbon atom of the 3 to 10 membered heterocycloaliphatic residue,

in which an “aliphatic group” and “aliphatic residue” can in each case be branched or unbranched, saturated or unsaturated,

in which a “cycloaliphatic residue” and a “heterocycloaliphatic residue” can in each case be saturated or unsaturated,

in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

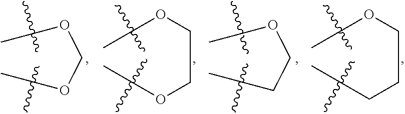 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

The compounds according to general formula (4-I) are known from WO 2012/205238 A1, which is fully included by reference.

Preferred embodiments of the compound according to general formula (I) have general formulae (4-Ia), (4-Ib), (4-Ic) or (4-Id),

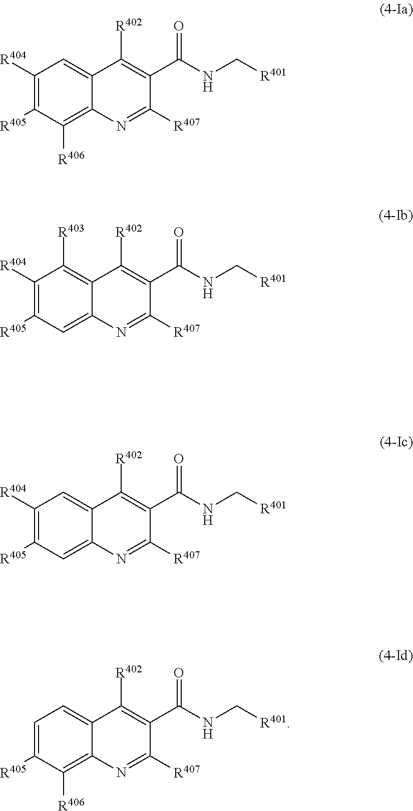

Another preferred embodiment of present invention is a compound according to general formula (4-I), wherein
R⁴⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

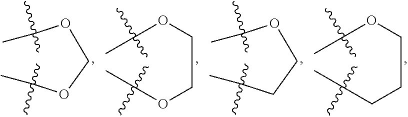 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH,
R⁴⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, preferably represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and an O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
R⁴⁰³, R⁴⁰⁴, R⁴⁰⁵ and R⁴⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R⁴⁰³, R⁴⁰⁴, R⁴⁰⁵ and R⁴⁰⁶ is ≠H,
R⁴⁰⁷ denotes a C₂₋₁₀-aliphatic residue, preferably a C₂₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
on the condition that if R⁴⁰⁷ denotes a 3 to 10 membered heterocycloaliphatic residue, the 3 to 10 membered heterocycloaliphatic residue is linked via a carbon atom.

In a preferred embodiment of the compound according to general formula (4-I), the residue
R⁴⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

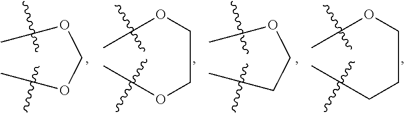 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH.

In a further preferred embodiment of the compound according to general formula (4-I), the residue
R⁴⁰¹ represents the partial structure (4-T1)

—(CR^{408a}R^{408b})ₘ₄—R^{408c} (4-T1),

wherein
m4 denotes 0, 1, 2, 3 or 4, preferably denotes 0, 1, or 2,
R^{408a} and R^{408b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O,
preferably each independently of one another represent H, F, Cl, Br, I, NH₂, a NH(C₁₋₄ aliphatic residue), a N(C₁₋₄ aliphatic residue)₂, OH, O—C₁₋₄ aliphatic residue or a C₁₋₄ aliphatic residue,
more preferably each independently of one another represent H, F, Cl, Br, I, an O—C₁₋₄ aliphatic residue or a C₁₋₄ aliphatic residue,
even more preferably each independently of one another represent H, F, an O—C₁₋₄ aliphatic residue or a C₁₋₄ aliphatic residue, and
R^{408c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes a C₃₋₁₀-cycloaliphatic residue or a 3 to 10 membered heterocycloaliphatic residue, preferably when m4 is 0, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   or denotes—preferably when m is =0—an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

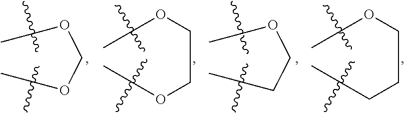 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R⁴⁰¹ represents the partial structure (4-T1),

wherein
m4 denotes 0, 1, or 2,
R^{408a} and R^{408b} each independently of one another represent H, F, Cl, Br, I, an O—C₁₋₄ aliphatic residue or a C₁₋₄ aliphatic residue,
preferably each independently of one another represent H, F, a O—C₁₋₂ aliphatic residue or a C₁₋₂ aliphatic residue, and
R^{408c} denotes a C₁₋₄ aliphatic residue, unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
or denotes—preferably when m4 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl or oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

More preferably,
R⁴⁰¹ represents the partial structure (4-T1),

wherein
m4 denotes 0, 1, or 2,
R^{408a} and R^{408b} each independently of one another represent H, F, Cl, Br, I, an O—C₁₋₄ aliphatic residue or a C₁₋₄ aliphatic residue,
preferably each independently of one another represent H, F, a O—C₁₋₂ aliphatic residue or a C₁₋₂ aliphatic residue, and
R^{408c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes—preferably when m4 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl,
   wherein benzyl, phenyl, thienyl and pyridyl, may in each case may be unsubstituted or mono- or polysubstituted, preferably unsubstituted or mono- or disubstituted with at least one substituent selected from the group consisting of F, CI, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In a further preferred embodiment of the compound according to general formula (4-I), the residue
R⁴⁰¹ represents the partial structure (4-T1),

wherein
m4 is 0, 1 or 2 and
R^{408a} and R^{408b} each independently of one another represent H, F, a O—C₁₋₄ aliphatic residue or a C₁₋₄ aliphatic residue; preferably H, F, CH₃ or OCH₃;
R^{408c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or

wherein
m4 is 0,
R^{408a} and R^{408b} each independently of one another represent H, F, a O—C₁₋₄ aliphatic residue or a C₁₋₄ aliphatic residue; preferably H, F, CH₃ or OCH₃; and
R^{408c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃.

Particularly preferred is a compound according to general formula (4-I) which has the following general formula (4-Ie),

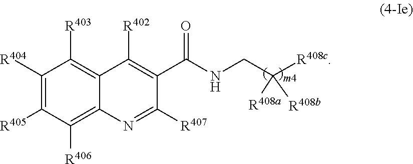

In a preferred embodiment of the compound according to general formula (4-I), the residue
R⁴⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted.

Preferably,
R⁴⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

More preferably,
R⁴⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
cyclopropyl, cyclobutyl, cyclopentyl, cyclohexyl, pyrrolidinyl, piperazinyl, 4-methylpiperazinyl, morpholinyl, or piperidinyl, preferably cyclopropyl, cyclobutyl, cyclopentyl or cyclohexyl, in each case unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted O—C₁₋₄-aliphatic residue.

Even more preferably,
R⁴⁰² represents H; F; Cl; Br; I; CN; CF₃; NO₂; OCF₃; SCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; S-Ethyl; cyclopropyl, cyclobutyl, cyclopentyl, and cyclohexyl.

Still more preferably,
R⁴⁰² is selected from the group consisting of H; F; Cl; CF₃; CN; SCF₃; OCF₃; CH₃; C₂H₅; n-propyl; iso-propyl; t-butyl; cyclopropyl; O—CH₃ and O—C₂H₅.

In particular,
R⁴⁰² is selected from the group consisting of H; F; Cl; CF₃; CH₃; C₂H₅, iso-propyl; cyclopropyl; and O—CH₃; preferably selected from the group consisting of H; F; Cl; CF₃; CH₃; and O—CH₃.

In a particular preferred embodiment of the compound according to general formula (4-I), the residue
R⁴⁰² is ≠H.

In a preferred embodiment of the compound according to general formula (4-I), the residues
R⁴⁰³, R⁴⁰⁴, R⁴⁰⁵ and R⁴⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R⁴⁰³, R⁴⁰⁴, R⁴⁰⁵ and R⁴⁰⁶ is ≠H.

Preferably,
R⁴⁰³, R⁴⁰⁴, R⁴⁰⁵ and R⁴⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a O—C(═O)—C₁₋₄-aliphatic residue, a S—C₁₋₄-aliphatic residue, a S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R⁴⁰³, R⁴⁰⁴, R⁴⁰⁵ and R⁴⁰⁶ is ≠H.

More preferably,
R⁴⁰³, R⁴⁰⁴, R⁴⁰⁵ and R⁴⁰⁶ each independently of one another represent H; F; Cl; Br; I; CN; CF₃; OCF₃; SCF₃; C(═O)H; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a S—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue, unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R⁴⁰³, R⁴⁰⁴, R⁴⁰⁵ and R⁴⁰⁶ is ≠H.

In a further preferred embodiment of the present invention
R⁴⁰³, R⁴⁰⁴, R⁴⁰⁵ and R⁴⁰⁶ each independently of one another are selected from the group consisting of H; F; Cl; Br; I; NO₂; CF₃; CN; OCF₃; SCF₃; a (C═O)—C₁₋₄aliphatic residue, a C₁₋₄aliphatic residue, O—C₁₋₄aliphatic residue, a S—C₁₋₄aliphatic residue, wherein the C₁₋₄-aliphatic residue in each case may be unsubstituted or mono- or polysubstituted with at least one substituent selected from the group consisting of F, Cl, and O—CH₃; on the condition that at least one of R⁴⁰³, R⁴⁰⁴, R⁴⁰⁵ and R⁴⁰⁶ is ≠H.

Preferably,
R⁴⁰³, R⁴⁰⁴, R⁴⁰⁵ and R⁴⁰⁶ each independently of one another are selected from the group consisting of H; F; Cl; Br; I; NO₂; CF₃; CN; OCF₃; SCF₃; methyl; ethyl; n-propyl; iso-propyl; n-butyl; sec.-butyl; tert.-butyl; cyclopropyl; C(═O)-methyl; C(═O)-ethyl; (C═O)-isopropyl; (C═O)-t-butyl; O-methyl; O-ethyl; O-isopropyl; O-t-butyl; O—(CH₂)₂—O—CH₃; S-Methyl; S-Ethyl;
on the condition that at least one of R⁴⁰³, R⁴⁰⁴, R⁴⁰⁵ and R⁴⁰⁶ is ≠H.

In particular,
R⁴⁰³, R⁴⁰⁴, R⁴⁰⁵ and R⁴⁰⁶ are each independently of one another are selected from the group consisting of H; F; Cl; Br; I; NO₂; CF₃; CN; (C═O)-methyl; (C═O)-ethyl; (C═O)-isopropyl; (C═O)-t-butyl; methyl; ethyl; isopropyl; t-butyl; O-methyl; O-Ethyl; O-isopropyl; O-t-butyl; OCF₃; S-methyl; S-ethyl; and SCF₃;
on the condition that at least one of R⁴⁰³, R⁴⁰⁴, R⁴⁰⁵ and R⁴⁰⁶ is ≠H.

More particularly,
R⁴⁰³, R⁴⁰⁴, R⁴⁰⁵ and R⁴⁰⁶ are each independently of one another selected from the group consisting of H; F; Cl; Br; CH₃; CF₃; CN; OCF₃ and NO₂;
on the condition that at least one of R⁴⁰³, R⁴⁰⁴, R⁴⁰⁵ and R⁴⁰⁶ is ≠H.

Most preferred,
R⁴⁰³, R⁴⁰⁴ and R⁴⁰⁶ each independently are selected from the group consisting of H and F; and
R⁴⁰⁵ denotes F; Cl; Br; CH₃; CF₃; OCF₃; CN; or NO₂; preferably denotes F, Br, CF₃ or CN.

In a particular preferred embodiment of the compound according to general formula (4-I) at least one of the residues R⁴⁰³, R⁴⁰⁴, R⁴⁰⁵ and R⁴⁰⁶ is ≠H.

In a preferred embodiment of the compound according to general formula (4-I), the residue
R⁴⁰⁷ denotes a C₂₋₁₀-aliphatic residue, preferably a C₂₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R⁴⁰⁷ denotes a 3 to 10 membered heterocycloaliphatic residue, the 3 to 10 membered heterocycloaliphatic residue is linked via a carbon atom.

In a further preferred embodiment of the compound according to general formula (4-I), the residue
R⁴⁰⁷ denotes a C₂₋₁₀-aliphatic residue, preferably a C₂₋₈-aliphatic residue, unsubstituted or mono- or polysubstituted with at least one substituent selected from the group consisting of F, Cl, Br, I, NO₂, a NH(C₁₋₄ aliphatic residue), a N(C₁₋₄ aliphatic residue)₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue.
on the condition that if R⁴⁰⁷ denotes a 3 to 10 membered heterocycloaliphatic residue, the 3 to 10 membered heterocycloaliphatic residue is linked via a carbon atom.

Preferably,
R⁴⁰⁷ denotes a C₂₋₈-aliphatic residue, unsubstituted or mono- or polysubstituted with at least one substituent selected from the group consisting of F, Cl, Br, I, OH, ═O, an O—C₁₋₄-aliphatic residue, OCF₃, a NH(C₁₋₄ aliphatic residue), a N(C₁₋₄ aliphatic residue)₂, SH, SCF₃, a S—C₁₋₄-aliphatic residue, a C(═O)—O—C₁₋₄-aliphatic residue, CF₃, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue.
on the condition that if R⁴⁰⁷ denotes a 3 to 10 membered heterocycloaliphatic residue, the 3 to 10 membered heterocycloaliphatic residue is linked via a carbon atom.

More preferably,
R⁴⁰⁷ denotes a C₂₋₈-aliphatic residue, preferably a C₂₋₆-aliphatic residue, unsubstituted or mono- or polysubstituted with at least one substituent selected from the group consisting of F, Cl, Br, I, OH, ═O, an O—C₁₋₄-aliphatic residue, OCF₃, a NH(C₁₋₄ aliphatic residue), a N(C₁₋₄ aliphatic residue)₂, SH, SCF₃, a S—C₁₋₄-aliphatic residue, CF₃, a C(═O)—O—C₁₋₄-aliphatic residue, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
on the condition that if R⁴⁰⁷ denotes a 3 to 10 membered heterocycloaliphatic residue, the 3 to 10 membered heterocycloaliphatic residue is linked via a carbon atom.

Even more preferably,
R⁴⁰⁷ denotes a C₂₋₈-aliphatic residue, preferably a C₂₋₆-aliphatic residue, unsubstituted or mono- or polysubstituted with at least one substituent selected from the group consisting of F, Cl, Br, I, OH, ═O, an O—C₁₋₄-aliphatic residue, OCF₃, an NH(C₁₋₄ aliphatic residue), an N(C₁₋₄ aliphatic residue)₂, SH, SCF₃, a S—C₁₋₄-aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue may in each case optionally bridged via a unsubstituted C₁₋₈ aliphatic group, preferably an unsubstituted C₁₋₄ aliphatic group,
on the condition that if R⁴⁰⁷ denotes a 3 to 10 membered heterocycloaliphatic residue, the 3 to 10 membered heterocycloaliphatic residue is linked via a carbon atom.

Still more preferably,
R⁴⁰⁷ denotes a C₂₋₆-aliphatic residue, unsubstituted or mono- or polysubstituted with at least one substituent selected from the group consisting of F, Cl, Br, I, OH, ═O, an O—C₁₋₄-aliphatic residue, an NH(C₁₋₄ aliphatic residue), an N(C₁₋₄ aliphatic residue)₂, a C(═O)—O—C₁₋₄-aliphatic residue, OCF₃, SH, SCF₃, a S—C₁₋₄-aliphatic residue, CF₃, and a C₁₋₄-aliphatic residue,
   wherein the C₁₋₄-aliphatic residue in each case is unsubstituted,
or denotes a C₃₋₁₀-cycloaliphatic residue, preferably a C₃₋₆-cycloaliphatic residue, or a 3 to 10 membered heterocycloaliphatic residue, preferably a 3 to 6 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with OH or an unsubstituted O—C₁₋₄-aliphatic residue,
and wherein the C₃₋₁₀-cycloaliphatic residue or the 3 to 10 membered heterocycloaliphatic residue may in each case optionally bridged via a unsubstituted C₁₋₄ aliphatic group,
on the condition that if R⁴⁰⁷ denotes a 3 to 10 membered heterocycloaliphatic residue, the 3 to 10 membered heterocycloaliphatic residue is linked via a carbon atom.

In particular,
R⁴⁰⁷ denotes a C₂₋₆-aliphatic residue, preferably selected from the group consisting of ethyl, n-propyl, 2-propyl, n-butyl, isobutyl, sec.-butyl, tert.-butyl, n-pentyl, isopentyl, neopentyl, n-hexyl, ethenyl and propenyl (—CH₂CH═CH₂, —CH═CH—CH₃, —C(═CH₂)—CH₃), unsubstituted or mono- or polysubstituted with at least one substituent selected from the group consisting of F, Cl, Br, I, OH, ═O, an O—C₁₋₄-aliphatic residue, an NH(C₁₋₄ aliphatic residue), an N(C₁₋₄ aliphatic residue)₂, a C(═O)—O—C₁₋₄-aliphatic residue, OCF₃, SH, SCF₃, a S—C₁₋₄-aliphatic residue, CF₃, and a C₁₋₄-aliphatic residue, preferably in each case unsubstituted or mono- or polysubstituted with at least one substituent selected from the group consisting of F, Cl, OH, an O—C₁₋₄-aliphatic residue, an NH(C₁₋₄ aliphatic residue), an N(C₁₋₄ aliphatic residue)₂, CF₃, and a C₁₋₄-aliphatic residue, more preferably in each case unsubstituted or mono- or polysubstituted with at least one substituent selected from the group consisting of F, Cl, OH, an O—C₁₋₄-aliphatic residue and an N(C₁₋₄ aliphatic residue)₂, even more preferably in each case unsubstituted or mono- or polysubstituted with at least one substituent selected from the group consisting of O-methyl, OH and N(methyl)₂,
   wherein the C₁₋₄-aliphatic residue in each case is unsubstituted,
or denotes a C₃₋₆-cycloaliphatic residue, preferably selected from the group consisting of cyclopropyl, cyclobutyl, cyclopentyl and cyclohexyl, or a 3 to 6 membered heterocycloaliphatic residue, preferably selected from the group consisting of piperidinyl (preferably piperidin-4-yl or piperidin-3-yl), tetrahydrofuranyl, and tetrahydropyranyl, in each case unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue, preferably in each case unsubstituted or mono- or polysubstituted with at least one substituent selected from the group consisting of F, Cl, OH, an O—C₁₋₄-aliphatic residue, CF₃, and a C₁₋₄-aliphatic residue, more preferably in each case unsubstituted or mono- or polysubstituted with at least one substituent selected from the group consisting of F, Cl, and an O—C₁₋₄-aliphatic residue, preferably O-methyl,
   wherein the C₁₋₄-aliphatic residue in each case is unsubstituted,
and wherein the C₃₋₆-cycloaliphatic residue or the 3 to 6 membered heterocycloaliphatic residue may in each case optionally bridged via an unsubstituted C₁₋₄ aliphatic group, preferably via an unsubstituted C₁₋₂ aliphatic group,
on the condition that if R⁴⁰⁷ denotes a 3 to 6 membered heterocycloaliphatic residue, the 3 to 6 membered heterocycloaliphatic residue is linked via a carbon atom.

Most preferred,
R⁴⁰⁷ denotes ethyl, n-propyl, 2-propyl, n-butyl, isobutyl, sec.-butyl, tert.-butyl, n-pentyl, isopentyl, neopentyl, n-hexyl, ethenyl or propenyl (—CH₂CH═CH₂, —CH═CH—CH₃, —C(═CH₂)—CH₃), in each case unsubstituted or mono- or polysubstituted with at least one substituent selected from the group consisting of F, Cl, OH, O—CH₃, CF₃, and N(CH₃)₂, preferably in each case unsubstituted or mono- or polysubstituted with at least one substituent selected from the group consisting of F, Cl, OH, O—CH₃, and N(CH₃)₂, more preferably in each case unsubstituted or mono- or polysubstituted with at least one substituent selected from the group consisting of O—CH₃, OH and N(CH₃)₂,
or denotes cyclopropyl, cyclobutyl, cyclopentyl and cyclohexyl, piperidinyl (preferably piperidin-4-yl or piperidin-3-yl), tetrahydrofuranyl, or tetrahydropyranyl, in each case unsubstituted or mono- or polysubstituted with at least one substituent selected from the group consisting of F, Cl, OH, an O—C₁₋₄-aliphatic residue, CF₃, and a C₁₋₄-aliphatic residue, preferably in each case unsubstituted or mono- or polysubstituted with at least one substituent selected from the group consisting of F, Cl, and an O—C₁₋₄-aliphatic residue, preferably O-methyl,
   wherein the C₁₋₄-aliphatic residue in each case is unsubstituted,
and wherein cyclopropyl, cyclobutyl, cyclopentyl and cyclohexyl, piperidinyl (preferably piperidin-4-yl or piperidin-3-yl), tetrahydrofuranyl, and tetrahydropyranyl may in each case be optionally bridged, preferably are bridged, via an unsubstituted C₁₋₄ aliphatic group, preferably via an unsubstituted C₁₋₂ aliphatic group,
on the condition that if R⁴⁰⁷ denotes piperidinyl tetrahydrofuranyl, or tetrahydropyranyl, piperidinyl tetrahydrofuranyl, or tetrahydropyranyl, each of these residues is linked via a carbon atom.

Particularly preferred is also a compound according to general formula (4-I), wherein
R⁴⁰¹ represents the partial structure (4-T1),

—(CR^{408a}R^{408b})ₘ₄—R^{408c} (4-T1),

wherein
m4 is 0, 1 or 2 and
R^{408a} and R^{408b} each independently of one another represent H, F, a O—C₁₋₄ aliphatic residue or a C₁₋₄ aliphatic residue; preferably H, F, CH₃ or OCH₃;
R^{408c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or

wherein
m4 is 0,
R^{408a} and R^{408b} each independently of one another represent H, F, a O—C₁₋₄ aliphatic residue or a C₁₋₄ aliphatic residue; preferably H, F, CH₃ or OCH₃; and
R^{408c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
R⁴⁰² is selected from the group consisting of H; F; Cl; CF₃; CH₃; C₂H₅, iso-propyl; cyclopropyl; and O—CH₃,
R⁴⁰³, R⁴⁰⁴, R⁴⁰⁵ and R⁴⁰⁶ are each independently of one another selected from the group consisting of H; F; Cl; CF₃; CN; OCF₃ and NO₂,
on the condition that at least one of R⁴⁰³, R⁴⁰⁴, R⁴⁰⁵ and R⁴⁰⁶ is ≠H,
R⁴⁰⁷ denotes ethyl, n-propyl, 2-propyl, n-butyl, isobutyl, sec.-butyl, tert.-butyl, n-pentyl, isopentyl, neopentyl, n-hexyl, ethenyl or propenyl (—CH₂CH═CH₂, —CH═CH—CH₃, —C(═CH₂)—CH₃), in each case unsubstituted or mono- or polysubstituted with at least one substituent selected from the group consisting of F, Cl, OH, O—CH₃, CF₃, and N(CH₃)₂, preferably in each case unsubstituted or mono- or polysubstituted with at least one substituent selected from the group consisting of F, Cl, OH, O—CH₃, and N(CH₃)₂, more preferably in each case unsubstituted or mono- or polysubstituted with at least one substituent selected from the group consisting of O—CH₃, OH and N(CH₃)₂,
or denotes cyclopropyl, cyclobutyl, cyclopentyl and cyclohexyl, piperidinyl (preferably piperidin-4-yl or piperidin-3-yl), tetrahydrofuranyl, or tetrahydropyranyl, in each case unsubstituted or mono- or polysubstituted with at least one substituent selected from the group consisting of F, Cl, OH, an O—C₁₋₄-aliphatic residue, CF₃, and a C₁₋₄-aliphatic residue, preferably in each case unsubstituted or mono- or polysubstituted with at least one substituent selected from the group consisting of F, Cl, and an O—C₁₋₄-aliphatic residue, preferably O-methyl,
   wherein the C₁₋₄-aliphatic residue in each case is unsubstituted,
and wherein cyclopropyl, cyclobutyl, cyclopentyl and cyclohexyl, piperidinyl (preferably piperidin-4-yl or piperidin-3-yl), tetrahydrofuranyl, and tetrahydropyranyl may in each case be optionally bridged via an unsubstituted C₁₋₄ aliphatic group, preferably via an unsubstituted C₁₋₂ aliphatic group,
on the condition that if R⁴⁰⁷ denotes piperidinyl, tetrahydrofuranyl, or tetrahydropyranyl, each of these residues is linked via a carbon atom.

Another embodiment of the present invention therefore relates to a compound of general formula (I), wherein the compound is selected from a compound according to general formula (5-I).

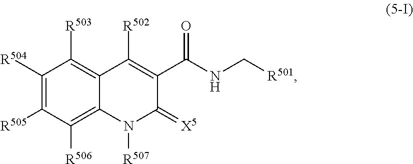

wherein
X⁵ denotes O or S, preferably O,
R⁵⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R⁵⁰² represents F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁵⁰³, R⁵⁰⁴, R⁵⁰⁵ and R⁵⁰⁶ each independently of one another represent H; F; Cl; Br; I; CN; CF₃; C(═O)H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄-aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂—C₁₋₄-aliphatic residue, a N(C₁₋₄ aliphatic residue)-C(═O)—C₁₋₄ aliphatic residue, or a N(C₁₋₄ aliphatic residue)-S(═O)₂—C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
preferably on the condition that at least one of R⁵⁰³, R⁵⁰⁴, R⁵⁰⁵ and R⁵⁰⁶ is ≠H,
R⁵⁰⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
on the condition that if R⁵⁰⁷ denotes a 3 to 10 membered heterocycloaliphatic residue, the 3 to 10 membered heterocycloaliphatic residue is linked via a carbon atom,

in which an “aliphatic group” and “aliphatic residue” can in each case be branched or unbranched, saturated or unsaturated,

in which a “cycloaliphatic residue” and a “heterocycloaliphatic residue” can in each case be saturated or unsaturated,

in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), a NH—C(═O)—C₁₋₄ aliphatic residue, a NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C_{1- 4}-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

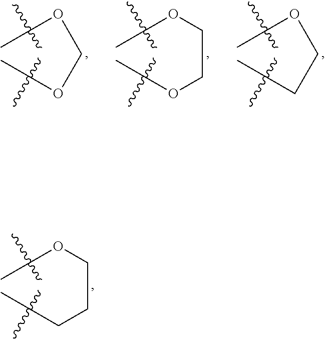 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

The compounds according to general formula (5-I) are known from WO 2012/025239 A1, which is fully included by reference.

Preferred embodiments of the compound according to general formula (5-I) have general formulae (5-I-I), and/or (5-I-II),

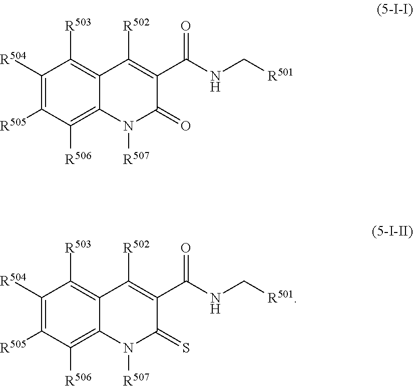

Particularly preferred is a compound according to general formula (5-I-I), i.e. a compound according to general formula (5-I), wherein X⁵ denotes O.

Further preferred embodiments of the compound according to general formula (I) have general formulae (5-Ia), (5-Ib), (5-Ic) and/or (5-Id),

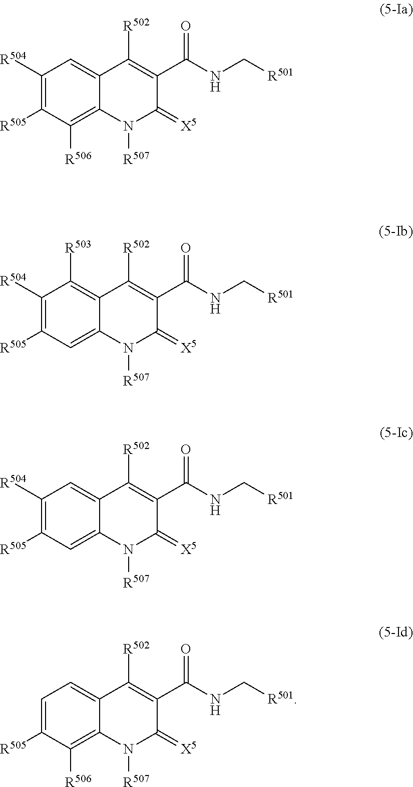

Another preferred embodiment of present invention is a compound according to general formula (5-I), wherein
R⁵⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄-aliphatic residue, OCF₃, SH, SCF₃, a S—C₁₋₄-aliphatic residue, CF₃, CN, a C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

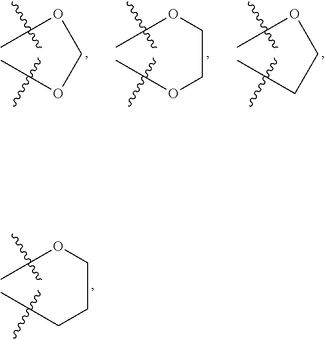 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), OH, an O—C₁₋₄ aliphatic residue, OCF₃, SH, SCF₃, a S—C₁₋₄ aliphatic residue, CF₃, CN and C(═O)—OH,
X⁵ represents O or S,
R⁵⁰² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, preferably represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and an O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, OH, an unsubstituted C₁₋₄-aliphatic residue and an unsubstituted O—C₁₋₄-aliphatic residue,
R⁵⁰³, R⁵⁰⁴, R⁵⁰⁵ and R⁵⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, a C₁₋₄-aliphatic residue and a O—C₁₋₄-aliphatic residue, and in each case optionally bridged via an unsubstituted C₁₋₄ aliphatic group, preferably on the condition that at least one of R⁵⁰³, R⁵⁰⁴, R⁵⁰⁵ and R⁵⁰⁶ is ≠H,
R⁵⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), OH, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁵⁰⁷ denotes a 3 to 10 membered heterocycloaliphatic residue, the 3 to 10 membered heterocycloaliphatic residue is linked via a carbon atom.

In a preferred embodiment of the compound according to general formula (5-I), the residue
R⁵⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or poly-substituted with at least one substituent selected from the group consisting of F, CI, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

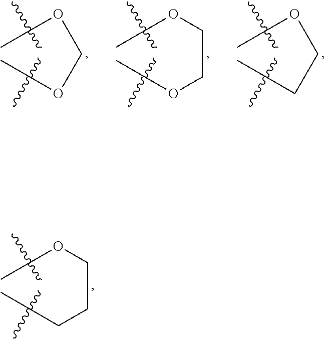 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), OH, an O—C₁₋₄ aliphatic residue, OCF₃, SH, SCF₃, a S—C₁₋₄ aliphatic residue, CF₃, CN and C(═O)—OH.

In a further preferred embodiment of the compound according to general formula (5-I), the residue
R⁵⁰¹ represents the partial structure (5-T1),

—(CR^{508a}R^{508b})ₘ₅—R^{508c} (5-T1),

wherein
m5 denotes 0, 1, 2, 3 or 4, preferably denotes 0, 1, or 2,
R^{508a} and R^{508b} each independently of one another represent H, F, Cl, Br, I, NO₂, NH₂, a NH(C₁₋₄ aliphatic residue), OH, an O—C₁₋₄ aliphatic residue, OCF₃, SH, SCF₃, a S—C₁₋₄ aliphatic residue, CF₃, CN, a C₁₋₄ aliphatic residue or C(═O)—OH,
preferably each independently of one another represent H, F, Cl, Br, I, NH₂, a NH(C₁₋₄ aliphatic residue), OH, O—C₁₋₄ aliphatic residue or a C₁₋₄ aliphatic residue,
more preferably each independently of one another represent H, F, Cl, Br, I, an O—C₁₋₄ aliphatic residue or a C₁₋₄ aliphatic residue,
even more preferably each independently of one another represent H, F, an O—C₁₋₄ aliphatic residue or a C₁₋₄ aliphatic residue, and
R^{508c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes a C₃₋₁₀-cycloaliphatic residue or a 3 to 10 membered heterocycloaliphatic residue, preferably when m is 0, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes—preferably when m5 is =0—an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

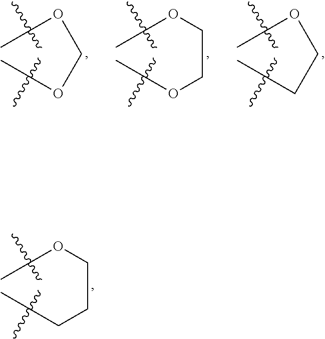 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R⁵⁰¹ represents the partial structure (5-T1),

wherein
m5 denotes 0, 1, or 2,
R^{508a} and R^{508b} each independently of one another represent H, F, Cl, Br, I, an O—C₁₋₄ aliphatic residue or a C₁₋₄ aliphatic residue,
preferably each independently of one another represent H, F, a O—C₁₋₂ aliphatic residue or a C₁₋₂ aliphatic residue, and
R^{508c} denotes a C₁₋₄ aliphatic residue, unsubstituted or mono- or polysubstituted with at least one substituent selected from the group consisting of F, Cl, Br, I, OH, an O—C₁₋₄ aliphatic residue, OCF₃, CF₃, a C₁₋₄-aliphatic residue and C(═O)—OH,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
or denotes—preferably when m5 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl or oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

More preferably,
R⁵⁰¹ represents the partial structure (5-T1),

wherein
m5 denotes 0, 1, or 2,
R^{508a} and R^{508b} each independently of one another represent H, F, Cl, Br, I, an O—C₁₋₄ aliphatic residue or a C₁₋₄ aliphatic residue,
preferably each independently of one another represent H, F, a O—C₁₋₂ aliphatic residue or a C₁₋₂ aliphatic residue, and
R^{508c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes—preferably when m5 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl,
   wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In a further preferred embodiment of the compound according to general formula (5-I), the residue
R⁵⁰¹ represents the partial structure (5-T1),

wherein
m5 is 0, 1 or 2 and
R^{508a} and R^{508b} each independently of one another represent H, F, a O—C₁₋₄ aliphatic residue or a C₁₋₄ aliphatic residue; preferably H, F, CH₃ or OCH₃;
R^{508c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or

wherein
m5 is 0,
R^{508a} and R^{508b} each independently of one another represent H, F, a O—C₁₋₄ aliphatic residue or a C₁₋₄ aliphatic residue; preferably H, F, CH₃ or OCH₃; and
R^{508c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃.

Particularly preferred is a compound according to general formula (5-I) which has the following general formula (5-le):

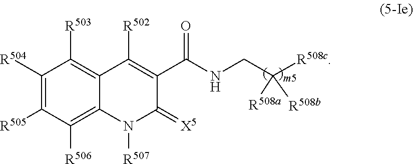

In particular,
R⁵⁰¹ represents aryl, preferably phenyl, or heteroaryl, preferably pyridyl or thienyl, in each case unsubstituted or mono- or disubstituted with at least one substituent selected from the group consisting of F, Cl, Br, I, OH, OCH₃, OCF₃, CF₃, CN, and CH₃,
preferably represents phenyl, unsubstituted or mono- or disubstituted with at least one substituent selected from the group consisting of F, Cl, Br, I, OH, OCH₃, OCF₃, CF₃, CN, and CH₃,

or represents an unsubstituted C₁₋₆-aliphatic residue.

In a further preferred embodiment of the compound according to general formula (5-I), the residue X⁵ denotes O.

In another preferred embodiment of the compound according to general formula (5-I), the residue X⁵ denotes S.

In a preferred embodiment of the compound according to general formula (5-I), the residue
R⁵⁰² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted.

Preferably,
R⁵⁰² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue,
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

More preferably,
R⁵⁰² represents F; Cl; Br; I; CN; CF₃; NO₂; OCF₃; SCF₃; a C₁₋₄-aliphatic residue, a S—C₁₋₄-aliphatic residue, or a O—C₁₋₄-aliphatic residue,
   wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue,
cyclopropyl, cyclobutyl, cyclopentyl, cyclohexyl, pyrrolidinyl, piperazinyl, 4-methylpiperazinyl, morpholinyl, or piperidinyl, preferably cyclopropyl, cyclobutyl, cyclopentyl or cyclohexyl, in each case unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted O—C₁₋₄-aliphatic residue.

Even more preferably,
R⁵⁰² represents F; Cl; Br; I; CN; CF₃; NO₂; OCF₃; SCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; S-Ethyl; cyclopropyl, cyclobutyl, cyclopentyl, and cyclohexyl.

Still more preferably,
R⁵⁰² is selected from the group consisting of F; Cl; Br; CF₃; CN; SCF₃; OCF₃; CH₃; C₂H₅; n-propyl; iso-propyl; t-butyl; cyclopropyl; O—CH₃ and O—C₂H₅.

In particular,
R⁵⁰² is selected from the group consisting of F; Cl; Br; CF₃; CH₃; C₂H₅, iso-propyl; cyclopropyl; and O—CH₃.

In a preferred embodiment of the compound according to general formula (5-I), the residues
R⁵⁰³, R⁵⁰⁴, R⁵⁰⁵ and R⁵⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, preferably on the condition that at least one of R⁵⁰³, R⁵⁰⁴, R⁵⁰⁵ and R⁵⁰⁶ is ≠H.

Preferably,
R⁵⁰³, R⁵⁰⁴, R⁵⁰⁵ and R⁵⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a O—C(═O)—C₁₋₄-aliphatic residue, a S—C₁₋₄-aliphatic residue, a S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, preferably on the condition that at least one of R⁵⁰³, R⁵⁰⁴, R⁵⁰⁵ and R⁵⁰⁶ is ≠H.

More preferably,
R⁵⁰³, R⁵⁰⁴, R⁵⁰⁵ and R⁵⁰⁶ each independently of one another represent H; F; Cl; Br; I; CN; CF₃; OCF₃; SCF₃; C(═O)H; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a S—C₁₋₄-aliphatic residue, wherein the C₁₋₄-aliphatic residue in each case may be unsubstituted or mono- or polysubstituted with at least one substituent selected from the group consisting of F, Cl, Br, I, OH, and a O—C₁₋₄-aliphatic residue; a C₃₋₆-cycloaliphatic residue, unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group,
preferably on the condition that at least one of R⁵⁰³, R⁵⁰⁴, R⁵⁰⁵ and R⁵⁰⁶ is ≠H.

In a further preferred embodiment of the present invention
R⁵⁰³, R⁵⁰⁴, R⁵⁰⁵ and R⁵⁰⁶ each independently of one another are selected from the group consisting of H; F; Cl; Br; I; NO₂; CF₃; CN; OCF₃; SCF₃; a (C═O)—C₁₋₄aliphatic residue, a C₁₋₄aliphatic residue, O—C₁₋₄ aliphatic residue, a S—C₁₋₄aliphatic residue, wherein the C₁₋₄-aliphatic residue in each case may be unsubstituted or mono- or polysubstituted with at least one substituent selected from the group consisting of F, Cl, OH, and O—CH₃;
preferably on the condition that at least one of R⁵⁰³, R⁵⁰⁴, R⁵⁰⁵ and R⁵⁰⁶ is ≠H.

Preferably,
R⁵⁰³, R⁵⁰⁴, R⁵⁰⁵ and R⁵⁰⁶ each independently of one another are selected from the group consisting of H; F; Cl; Br; I; NO₂; CF₃; CN; OCF₃; SCF₃; methyl; ethyl; n-propyl; iso-propyl; n-butyl; sec.-butyl; tert-butyl; cyclopropyl; C(═O)-methyl; C(═O)-ethyl; (C═O)-isopropyl; (C═O)-t-butyl; O-methyl; O-ethyl; O-isopropyl; O-t-butyl; O—(CH₂)₂—O—CH₃; S-Methyl; S-Ethyl;
preferably on the condition that at least one of R⁵⁰³, R⁵⁰⁴, R⁵⁰⁵ and R⁵⁰⁶ is ≠H.

In particular,
R⁵⁰³, R⁵⁰⁴, R⁵⁰⁵ and R⁵⁰⁶ are each independently of one another are selected from the group consisting of H; F; Cl; Br; I; NO₂; CF₃; CN; (C═O)-methyl; (C═O)-ethyl; (C═O)-isopropyl; (C═O)-t-butyl; methyl; ethyl; isopropyl; t-butyl; O-methyl; O-Ethyl; O-isopropyl; O-t-butyl; OCF₃; S-methyl; S-ethyl; and SCF₃;
preferably on the condition that at least one of R⁵⁰³, R⁵⁰⁴, R⁵⁰⁵ and R⁵⁰⁶ is ≠H.

More particularly,
R⁵⁰³, R⁵⁰⁴, R⁵⁰⁵ and R⁵⁰⁶ are each independently of one another selected from the group consisting of H; F; Cl; Br; CF₃; CN; OCF₃ and NO₂;
preferably on the condition that at least one of R⁵⁰³, R⁵⁰⁴, R⁵⁰⁵ and R⁵⁰⁶ is ≠H.

Most preferred,
R⁵⁰³, R⁵⁰⁴ and R⁵⁰⁶ each independently of one another represent H or F, preferably each denotes H; and
R⁵⁰⁵ denotes H; F; Cl; Br; CF₃; OCF₃; CN; or NO₂; preferably denotes F; Cl; Br; CF₃; OCF₃; or CN.

In a particular preferred embodiment of the compound according to general formula (5-I) at least one of the residues R⁵⁰³, R⁵⁰⁴, R⁵⁰⁵ and R⁵⁰⁶ is ≠H.

In another particular preferred embodiment of the compound according to general formula (5-I) at least two of the residues R⁵⁰³, R⁵⁰⁴, R⁵⁰⁵ and R⁵⁰⁶ denote H, preferably at least two of R⁵⁰³, R⁵⁰⁴ and R⁵⁰⁶ denote H.

In a preferred embodiment of the compound according to general formula (5-I), the residue
R⁵⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R⁵⁰⁷ denotes a 3 to 10 membered heterocycloaliphatic residue, the 3 to 10 membered heterocycloaliphatic residue is linked via a carbon atom.

In a further preferred embodiment of the compound according to general formula (5-I), the residue
R⁵⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, OH, an O—C₁₋₄-aliphatic residue, OCF₃, SH, SCF₃, a S—C₁₋₄-aliphatic residue, a C(═O)—O—C₁₋₄-aliphatic residue, COOH, CF₃, CN, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
and wherein the C₃₋₁₀-cycloaliphatic residue or the 3 to 10 membered heterocycloaliphatic residue may in each case be optionally bridged, preferably is bridged, via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue,
on the condition that if R⁵⁰⁷ denotes a 3 to 10 membered heterocycloaliphatic residue, the 3 to 10 membered heterocycloaliphatic residue is linked via a carbon atom.

Preferably,
R⁵⁰⁷ denotes a C₁₋₈-aliphatic residue, unsubstituted or mono- or polysubstituted with at least one substituent selected from the group consisting of F, Cl, Br, I, OH, an O—C₁₋₄-aliphatic residue, OCF₃, SH, SCF₃, a S—C₁₋₄-aliphatic residue, a C(═O)—O—C₁₋₄-aliphatic residue, COOH, CF₃, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
on the condition that if R⁵⁰⁷ denotes a 3 to 10 membered heterocycloaliphatic residue, the 3 to 10 membered heterocycloaliphatic residue is linked via a carbon atom.

More preferably,
R⁵⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, an O—C₁₋₄-aliphatic residue, OCF₃, SH, SCF₃, a S—C₁₋₄-aliphatic residue, COOH, CF₃, a C(═O)—O—C₁₋₄-aliphatic residue, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue in each case may be bridged, preferably is bridged, via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
on the condition that if R⁵⁰⁷ denotes a 3 to 10 membered heterocycloaliphatic residue, the 3 to 10 membered heterocycloaliphatic residue is linked via a carbon atom.

Even more preferably,
R⁵⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, an O—C₁₋₄-aliphatic residue, OCF₃, SH, SCF₃, a S—C₁₋₄-aliphatic residue, COOH, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue may in each case be bridged, preferably is bridged, via a unsubstituted C₁₋₈ aliphatic group, preferably an unsubstituted C₁₋₄ aliphatic group,
on the condition that if R⁵⁰⁷ denotes a 3 to 10 membered heterocycloaliphatic residue, the 3 to 10 membered heterocycloaliphatic residue is linked via a carbon atom.

Still more preferably,
R⁵⁰⁷ denotes a C₁₋₆-aliphatic residue, unsubstituted or mono- or polysubstituted with at least one substituent selected from the group consisting of F, Cl, Br, I, OH, an O—C₁₋₄-aliphatic residue, COOH, a C(═O)—O—C₁₋₄-aliphatic residue, OCF₃, SH, SCF₃, a S—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with OH or an unsubstituted O—C₁₋₄-aliphatic residue.
and wherein the C₃₋₁₀-cycloaliphatic residue or the 3 to 10 membered heterocycloaliphatic residue in each case may be bridged, preferably is bridged, via a unsubstituted C₁₋₄ aliphatic group,
on the condition that if R⁵⁰⁷ denotes a 3 to 10 membered heterocycloaliphatic residue, the 3 to 10 membered heterocycloaliphatic residue is linked via a carbon atom.

In particular,
R⁵⁰⁷ denotes a C₁₋₆-aliphatic residue, unsubstituted or mono- or polysubstituted with at least one substituent selected from the group consisting of F, Cl, Br, I, OH, an O—C₁₋₄-aliphatic residue, COOH, a C(═O)—O—C₁₋₄-aliphatic residue, OCF₃, SH, SCF₃, a S—C₁₋₄-aliphatic residue, CF₃, and a C₁₋₄-aliphatic residue, preferably unsubstituted or mono- or polysubstituted with at least one substituent selected from the group consisting of F, Cl, OH, an O—C₁₋₄-aliphatic residue, OCF₃, CF₃, and a C₁₋₄-aliphatic residue,
   wherein the C₁₋₄-aliphatic residue in each case may be unsubstituted or mono- or polysubstituted with at least one substituent selected from the group consisting of OH, and an unsubstituted O—C₁₋₄-aliphatic residue,
or denotes a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
on the condition that if R⁵⁰⁷ denotes a 3 to 6 membered heterocycloaliphatic residue, the 3 to 6 membered heterocycloaliphatic residue is linked via a carbon atom.

Most preferred,
R⁵⁰⁷ denotes a C₁₋₆-aliphatic residue, unsubstituted or mono- or polysubstituted with at least one substituent selected from the group consisting of F, Cl, OH, a C(═O)—O—C₁₋₄-aliphatic residue, COOH and an O—C₁₋₄-aliphatic residue, preferably at least one substituent selected from the group consisting of OH, a C(═O)—O—C₁₋₄-aliphatic residue, COOH and an O—C₁₋₄-aliphatic residue,
   wherein the C₁₋₄-aliphatic residue in each case may be unsubstituted or mono- or polysubstituted with at least one substituent selected from the group consisting of OH, and an unsubstituted O—C₁₋₄-aliphatic residue,
or denotes a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, ═O, an O—C₁₋₄ aliphatic residue, a C(═O)—O—C₁₋₄-aliphatic residue, CF₃, and a C₁₋₄-aliphatic residue,
on the condition that if R⁵⁰⁷ denotes a 3 to 6 membered heterocycloaliphatic residue, the 3 to 6 membered heterocycloaliphatic residue is linked via a carbon atom.

In particular most preferred
R⁵⁰⁷ is selected from the group consisting of CH₃, C₂H₅, C₃H₇, C₄H₉, C₅H₁₁, CH(CH₃)₂, C₂H₄—CH(CH₃)₂, C₃H₆—CH(CH₃)₂, CH₂OH, C₂H₄OH, CH₂—OCH₃, C₂H₄—OCH₃, C₃H₆—OCH₃, CH₂—O—C₂H₅, C₂H₄—O—C₂H₅, CH₂—CH(CH₃)(OCH₃), CH₂—CH(C₂H₅)(OCH₃), CH(CH₃)(CH₂—OCH₃), CH(C₂H₄—OCH₃)₂, CH(C₂H₅)(CH₂—OCH₃), C₂H₄—O—C₂H₄—O—CH₃, CH₂—O—C₂H₄—O—CH₃, C₂H₄—O—C₂H₄—OH, CH₂—O—C₂H₄—OH, CH₂—C(═O)O—CH₃, C₂H₄—C(═O)OCH₃, C₂H₄—C(═O)OH, CH₂—C(═O)OH,
or denotes an unsubstituted C₃₋₆-cycloaliphatic residue or an unsubstituted 3 to 6 membered heterocycloaliphatic residue, preferably tetrahydropyranyl or piperidinyl.

Particularly preferred is also a compound according to general formula (I), wherein
R⁵⁰¹ represents the partial structure (5-T1),

—(CR^{508a}R^{508b})ₘ₅—R^{508c} (5-T1),

wherein
m5 is 0, 1 or 2 and
R^{508a} and R^{508b} each independently of one another represent H, F, a O—C₁₋₄ aliphatic residue or a C₁₋₄ aliphatic residue; preferably H, F, CH₃ or OCH₃;
R^{508c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or

wherein
m5 is 0,
R^{508a} and R^{508b} each independently of one another represent H, F, a O—C₁₋₄ aliphatic residue or a C₁₋₄ aliphatic residue; preferably H, F, CH₃ or OCH₃; and
R^{508c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
X⁵ represents O or S, preferably O,
R⁵⁰² is selected from the group consisting of F; Cl; Br; CF₃; CH₃; C₂H₅, iso-propyl; cyclopropyl; and O—CH₃,
R⁵⁰³, R⁵⁰⁴, R⁵⁰⁵ and R⁵⁰⁶ are each independently of one another selected from the group consisting of H; F; Cl; Br; CF₃; CN; OCF₃ and NO₂;
preferably on the condition that at least one of R⁵⁰³, R⁵⁰⁴, R⁵⁰⁵ and R⁵⁰⁶ is ≠H,
R⁵⁰⁷ denotes a C₁₋₆-aliphatic residue, unsubstituted or mono- or polysubstituted with at least one substituent selected from the group consisting of F, Cl, Br, I, OH, an O—C₁₋₄-aliphatic residue, COOH, a C(═O)—O—C₁₋₄-aliphatic residue, OCF₃, SH, SCF₃, a S—C₁₋₄-aliphatic residue, CF₃, and a C₁₋₄-aliphatic residue, preferably unsubstituted or mono- or polysubstituted with at least one substituent selected from the group consisting of F, Cl, OH, an O—C₁₋₄-aliphatic residue, OCF₃, CF₃, and a C₁₋₄-aliphatic residue,
   wherein the C₁₋₄-aliphatic residue in each case may be unsubstituted or mono- or polysubstituted with at least one substituent selected from the group consisting of OH, and an unsubstituted O—C₁₋₄-aliphatic residue,
or denotes an unsubstituted C₃₋₆-cycloaliphatic residue or an unsubstituted 3 to 6 membered heterocycloaliphatic residue,
on the condition that if R⁵⁰⁷ denotes a 3 to 6 membered heterocycloaliphatic residue, the 3 to 6 membered heterocycloaliphatic residue is linked via a carbon atom.

Particularly preferred is also a compound according to general formula (5-I), wherein
R⁵⁰¹ represents aryl, preferably phenyl, or heteroaryl, preferably pyridyl or thienyl, in each case unsubstituted or mono- or disubstituted with at least one substituent selected from the group consisting of F, Cl, Br, I, OH, OCH₃, OCF₃, CF₃, CN, and CH₃,
preferably represents phenyl, unsubstituted or mono- or disubstituted with at least one substituent selected from the group consisting of F, Cl, Br, I, OH, OCH₃, OCF₃, CF₃, CN, and CH₃;
X⁵ is O;
R⁵⁰² is selected from the group consisting of F, Cl, CF₃, CH₃, C₂H₅, iso-propyl, cyclopropyl, and O—CH₃; preferably is selected from the group consisting of CH₃, C₂H₅, OCH₃ and CF₃;
R⁵⁰³, R⁵⁰⁴, R⁵⁰⁵ and R⁵⁰⁶ are each independently of one another selected from the group consisting of H, F, Cl, Br, CF₃, CN, OCF₃ and NO₂;
preferably on the condition that at least one of R⁵⁰³, R⁵⁰⁴, R⁵⁰⁵ and R⁵⁰⁶ is ≠H, more
preferably on the condition that R⁵⁰⁵ is ≠H;
R⁵⁰⁷ denotes a saturated C₁₋₆-aliphatic residue, unsubstituted or mono- or disubstituted with at least one substituent selected from the group consisting of F, Cl, Br, I, OH, O—CH₃, O—C₂H₅, OCF₃, OC₂H₄—O—CH₃, COOH, C(═O)OCH₃, SCF₃, and CF₃,
preferably denotes a saturated C₁₋₆-aliphatic residue, unsubstituted or mono- or disubstituted with at least one substituent selected from the group consisting of OH, O—CH₃, O—C₂H₅, OCF₃, OC₂H₄—O—CH₃, COOH, and C(═O)OCH₃.

Another embodiment of the present invention therefore relates to a compound of general formula (I), wherein the compound is selected from a compound according to general formula (6-I).

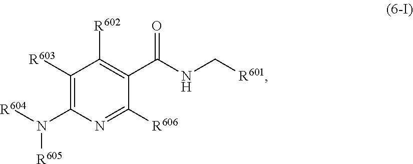

wherein
R⁶⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R⁶⁰² represents F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁶⁰³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁶⁰⁴ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged, preferably in each case bridged, via a C₁₋₈ aliphatic group, which in turn may be unsubstituted or mono- or polysubstituted;
on the condition that if R⁶⁰⁴ denotes a 3 to 10 membered heterocycloaliphatic residue or a heteroaryl, the 3 to 10 membered heterocycloaliphatic residue or the heteroaryl is linked via a carbon atom;
R⁶⁰⁵ denotes H or a C₁₋₁₀-aliphatic residue, preferably a C₁₋₄-aliphatic residue, unsubstituted or mono- or polysubstituted; or
R⁶⁰⁴ and R⁶⁰⁵ form together with the nitrogen atom connecting them a 3 to 10 membered heterocycloaliphatic residue, preferably a 4 to 7 membered heterocycloaliphatic residue, unsubstituted or mono- or polysubstituted, which may optionally be condensed with aryl or heteroaryl, preferably selected from the group consisting of phenyl, pyridyl and thienyl, wherein the aryl or heteroaryl residues condensed in this way can for their part be respectively unsubstituted or mono- or polysubstituted;
R⁶⁰⁶ represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁶⁰⁶ denotes a 3 to 10 membered heterocycloaliphatic residue, the 3 to 10 membered heterocycloaliphatic residue is linked via a carbon atom, or
denotes S—R⁶⁰⁷, O—R⁶⁰⁸ or N(R⁶⁰⁹R⁶¹⁰),
wherein
   R⁶⁰⁷ and R⁶⁰⁸ in each case represent a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
   on the condition that if R⁶⁰⁷ or R⁶⁰⁸ denotes a 3 to 10 membered heterocycloaliphatic residue, the 3 to 10 membered heterocycloaliphatic residue is linked via a carbon atom,
   R⁶⁰⁹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
   on the condition that if R⁶⁰⁹ denotes a 3 to 10 membered heterocycloaliphatic residue, the 3 to 10 membered heterocycloaliphatic residue is linked via a carbon atom;
   R⁶¹⁰ denotes H or a C₁₋₁₀-aliphatic residue, preferably a C₁₋₄-aliphatic residue, unsubstituted or mono- or polysubstituted; or
   R⁶⁰⁹ and R⁶¹⁰ form together with the nitrogen atom connecting them a 3 to 10 membered heterocycloaliphatic residue, preferably a 4 to 7 membered heterocycloaliphatic residue, unsubstituted or mono- or polysubstituted; which may optionally be condensed with aryl or heteroaryl, wherein the aryl or heteroaryl residues condensed in this way can for their part be respectively unsubstituted or mono- or polysubstituted;

in which an “aliphatic group” and an “aliphatic residue” can in each case be branched or unbranched, saturated or unsaturated,

in which a “cycloaliphatic residue” and a “heterocycloaliphatic residue” can in each case be saturated or unsaturated,

in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C_{1- 4}-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to “aryl” and a “heteroaryl” relates, with respect to the corresponding residues, to the substitution of one or more hydrogen atoms each independently of one another by at least one substituent selected from the group consisting of F, C, Br, NO₂, NH₂,

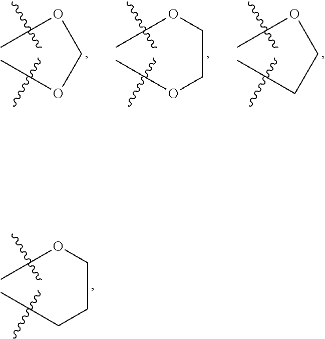 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

In an particularly preferred embodiment of the compound according to general formula (6-I) radicals R⁶⁰¹, R⁶⁰², R⁶⁰³, R⁶⁰⁴, R⁶⁰⁵ and R⁶⁰⁶ have the meanings described herein in connection with the compounds according to the invention and preferred embodiments thereof, with the proviso that R⁶⁰¹ comprises at least 4 atoms selected from the group consisting of carbon and heteroatoms, preferably at least 4 atoms selected from the group consisting of carbon atoms and heteroatoms selected from the group consisting of N, O and S.

The compounds according to general formula (6-I) are known from WO 2012/052167 A1, which is fully included by reference.

The present invention further relates to a substituted compound of general formula (6-I),

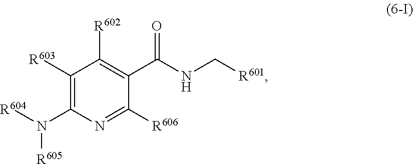

wherein
R⁶⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R⁶⁰² represents F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁶⁰³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁶⁰⁴ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged, preferably in each case bridged, via a C₁₋₈ aliphatic group, which in turn may be unsubstituted or mono- or polysubstituted;
on the condition that if R⁶⁰⁴ denotes a 3 to 10 membered heterocycloaliphatic residue or a heteroaryl, the 3 to 10 membered heterocycloaliphatic residue or the heteroaryl is linked via a carbon atom;
R⁶⁰⁵ denotes H or a C₁₋₁₀-aliphatic residue, preferably a C₁₋₄-aliphatic residue, unsubstituted or mono- or polysubstituted; or
R⁶⁰⁴ and R⁶⁰⁵ form together with the nitrogen atom connecting them a 3 to 10 membered heterocycloaliphatic residue, preferably a 4 to 7 membered heterocycloaliphatic residue, unsubstituted or mono- or polysubstituted,
R⁶⁰⁶ represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
on the condition that if R⁶⁰⁶ denotes a 3 to 10 membered heterocycloaliphatic residue, the 3 to 10 membered heterocycloaliphatic residue is linked via a carbon atom, or
denotes S—R⁶⁰⁷, O—R⁶⁰⁸ or N(R⁶⁰⁹R⁶¹⁰),
wherein
   R⁶⁰⁷ and R⁶⁰⁸ in each case represent a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
   on the condition that if R⁶⁰⁷ or R⁶⁰⁸ denotes a 3 to 10 membered heterocycloaliphatic residue, the 3 to 10 membered heterocycloaliphatic residue is linked via a carbon atom,
   R⁶⁰⁹ represents a C₁₋₁₀-aliphatic residue, unsubstituted or mono- or poly-substituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group,
   which in turn may be unsubstituted or mono- or polysubstituted;
   on the condition that if R⁶⁰⁹ denotes a 3 to 10 membered heterocycloaliphatic residue, the 3 to 10 membered heterocycloaliphatic residue is linked via a carbon atom;
   R⁶¹⁰ denotes a C₁₋₁₀-aliphatic residue, preferably a C₁₋₄-aliphatic residue, unsubstituted or mono- or polysubstituted; or
   R⁶⁰⁹ and R⁶¹⁰ form together with the nitrogen atom connecting them a 3 to 10 membered heterocycloaliphatic residue, preferably a 4 to 7 membered heterocycloaliphatic residue, unsubstituted or mono- or polysubstituted;

in which an “aliphatic group” and an “aliphatic residue” can in each case be branched or unbranched, saturated or unsaturated,

in which a “cycloaliphatic residue” and a “heterocycloaliphatic residue” can in each case be saturated or unsaturated,

in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C_{1- 4}-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

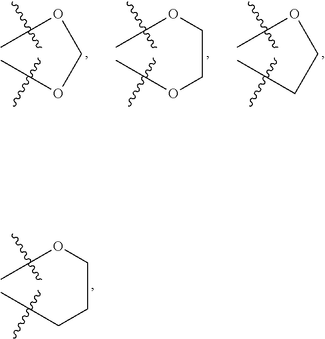 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

In another preferred embodiment of the compound according to formula (6-I), preferred substituents of “cycloaliphatic residue” and “heterocycloaliphatic residue” are selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, CH₂OH, CH₂—OCH₃, C₂H₄—OH, C₂H₄—OCH₃ CH₂—CF₃, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂.

In another preferred embodiment of the compound according to formula (6-I), preferred substituents of “aryl” and “heteroaryl” are selected from the group consisting of F, Cl, Br, I, NO₂, NH₂,

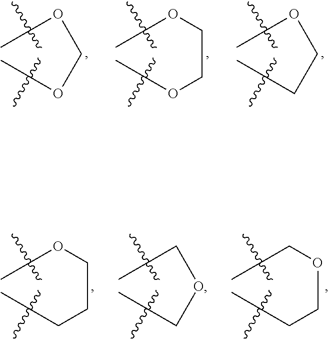 [SMILES: *OCO* ; *OCCO* ; *CCO* ; *CCCO* ; *COC* ; *CCOC*]

an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCFH₂, OCF₂H, OCF₃, a O—C₁₋₄-aliphatic residue, SH, SCF₃, a S—C₁₋₄-aliphatic residue, S(═O)₂OH, a S(═O)₂—C₁₋₄-aliphatic residue, a S(═O)₂—NH—C₁₋₄-aliphatic residue, CN, CF₃, CF₂H, CHF₂, a C₁₋₄-aliphatic residue, CH₂OH, CH₂—OCH₃, C₂H₄—OH, C₂H₄—OCH₃, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, CONH₂, a C(═O)—NH(C₁₋₄ aliphatic residue), a C(═O)—N(C₁₋₄ aliphatic residue)₂, aryl, preferably phenyl, or benzyl, in each case unsubstituted or mono- or polysubstituted with at least one substituent selected from the group consisting of F, Cl, Br, I, CN, CF₃, CH₃, C₂H₅, iso-propyl, tert.-butyl, C(═O)—OH, C(═O)—CH₃, C(═O)—C₂H₅, C(═O)—O—CH₃ and C(═O)—O—C₂H₅, O—CH₃, OCF₃, O—CH₂—OH, O—CH₂—O—CH₃, SH, S—CH₃, SCF₃, NO₂, NH₂, N(CH₃)₂, N(CH₃)(C₂H₅) and N(C₂H₅)₂, heteroaryl, preferably pyridyl, thienyl, furyl, thiazolyl or oxazolyl, in each case unsubstituted or mono- or polysubstituted with at least one substituent selected from the group consisting of F, Cl, Br, I, CN, CF₃, CH₃, C₂H₅, iso-propyl, tert.-butyl, C(═O)—OH, C(═O)—CH₃, C(═O)—C₂H₅, C(═O)—O—CH₃ and C(═O)—O—C₂H₅, O—CH₃, OCF₃, O—CH₂—OH, O—CH₂—O—CH₃, SH, S—CH₃, SCF₃, NO₂, NH₂, N(CH₃)₂, N(CH₃)(C₂H₅) and N(C₂H₅)₂.

In yet another preferred embodiment of the compound according to general formula (6-I) the particular radicals R⁶⁰¹-R⁶⁰⁵ have the meanings described herein in connection with the compounds according to the invention and preferred embodiments thereof and
R⁶⁰⁶ represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁶⁰⁶ denotes a 3 to 10 membered heterocycloaliphatic residue, the 3 to 10 membered heterocycloaliphatic residue is linked via a carbon atom, or
denotes S—R⁶⁰⁷ or O—R⁶⁰⁸
wherein R⁶⁰⁷ and R⁶⁰⁸ in each case represent a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
on the condition that if R⁶⁰⁷ or R⁶⁰⁸ denotes a 3 to 10 membered heterocycloaliphatic residue, the 3 to 10 membered heterocycloaliphatic residue is linked via a carbon atom.

In another preferred embodiment of the present invention the compound according to general formula (6-I) has the general formula (6-I-a)

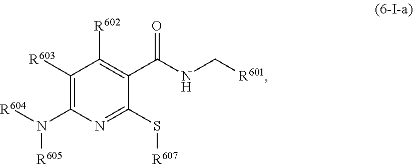

wherein

the particular radicals R⁶⁰¹-R⁶⁰⁵ and R⁶⁰⁷ have the meanings described herein in connection with the compounds according to the invention and preferred embodiments thereof.

In another preferred embodiment of the present invention the compound according to general formula (6-I) has the general formula (6-I-b)

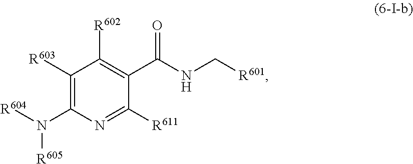

wherein the particular radicals R⁶⁰¹-R⁶⁰⁵ have the meanings described herein in connection with the compounds according to the invention and preferred embodiments thereof, and wherein
R⁶¹¹ represents O—R⁶⁰⁸ or N(R⁶⁰⁹R⁶¹⁰),
   wherein R⁶⁰⁸, R⁶⁰⁹ and R⁶¹⁰ have the meanings described herein in connection with the compounds according to the invention and preferred embodiments thereof,
or represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
on the condition that if R⁶¹¹ denotes a 3 to 10 membered heterocycloaliphatic residue, the binding is carried out via a carbon atom of the 3 to 10 membered heterocycloaliphatic residue.

In a particular preferred embodiment of the present invention, radical R⁶¹¹ in general formula (6-I-b) and radical R⁶⁰⁶ in general formula (6-I) represents O—R⁶⁰⁸, wherein R⁶⁰⁸ has the meanings described herein in connection with the compounds according to the invention and preferred embodiments thereof.

In another particular preferred embodiment of the present invention, radical R⁶¹¹ in general formula (6-I-b) and radical R⁶⁰⁶ in general formula (6-I) represents N(R⁶⁰⁹R⁶¹⁰), wherein R⁶⁰⁹ and R⁶¹⁰ have the meanings described herein in connection with the compounds according to the invention and preferred embodiments thereof.

In yet another particular preferred embodiment of the present invention, radical R⁶¹¹ in general formula (6-I-b) and radical R⁶⁰⁶ in general formula (6-I) represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted,

on the condition that if R⁶¹¹ denotes a 3 to 10 membered heterocycloaliphatic residue, the binding is carried out via a carbon atom of the 3 to 10 membered heterocycloaliphatic residue.

Another preferred embodiment of the compound according to general formula (6-I) has the general formula (6-I-c),

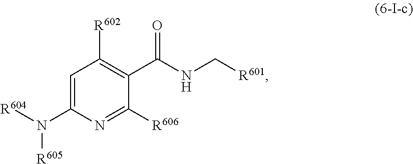

wherein the particular radicals R⁶⁰¹, R⁶⁰², R⁶⁰⁴, R⁶⁰⁵ and R⁶⁰⁶ have the meanings described herein in connection with the compounds according to the invention and preferred embodiments thereof.

Another preferred embodiment of the compound according to general formula (6-I) has the general formula (6-I-e) or (6-I-f),

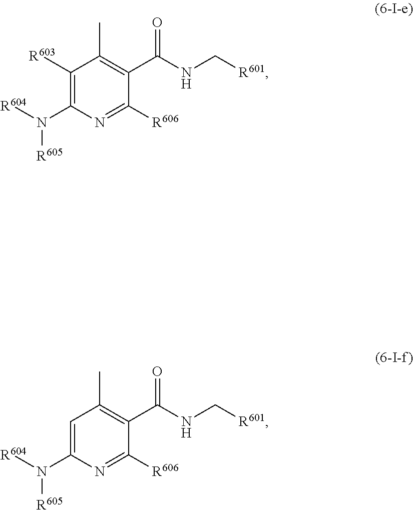

wherein the particular radicals R⁶⁰¹, R⁶⁰³, R⁶⁰⁴, R⁶⁰⁵ and R⁶⁰⁶ or R⁶⁰¹, R⁶⁰⁴, R⁶⁰⁵ and R⁶⁰⁶, respectively, have the meanings described herein in connection with the compounds according to the invention and preferred embodiments thereof.

In yet another preferred embodiment of the compound according to general formula (6-I) radicals R⁶⁰², R⁶⁰³, R⁶⁰⁴, R⁶⁰⁵ and R⁶⁰⁶ have the meanings described herein in connection with the compounds according to the invention and preferred embodiments thereof, and R⁶⁰¹ represents aryl or heteroaryl, in each case unsubstituted or mono- or polysubstituted.

In case R⁶⁰⁴ and R⁶⁰⁵ of the compound of general formula (6-I) form together with the nitrogen atom connecting them a 3 to 10 membered heterocycloaliphatic residue, preferably a 4 to 7 membered heterocycloaliphatic residue, unsubstituted or mono- or polysubstituted, said heterocycloaliphatic residue may optionally be condensed with aryl or heteroaryl or with a C₃₋₁₀ cycloaliphatic residue or with a 3 to 10 membered heterocycloaliphatic residue, wherein the aryl, heteroaryl, C₃₋₁₀ cycloaliphatic or 3 to 10 membered heterocycloaliphatic residues condensed in this way can for their part be respectively unsubstituted or mono- or polysubstituted. Preferably, said heterocycloaliphatic residue formed by R⁶⁰⁴ and R⁶⁰⁵ of the compound of general formula (6-I) together with the nitrogen atom connecting them may optionally be condensed with aryl or heteroaryl, wherein the aryl, or heteroaryl, preferably selected from the group consisting of phenyl, pyridyl and thienyl condensed in this way can for their part be respectively unsubstituted or mono- or polysubstituted.

Particularly preferably, in case R⁶⁰⁴ and R⁶⁰⁵ form together with the nitrogen atom connecting them a 3 to 10 membered heterocycloaliphatic residue, preferably a 4 to 7 membered heterocycloaliphatic residue, unsubstituted or mono- or polysubstituted, said heterocycloaliphatic residue may optionally be condensed with aryl or heteroaryl, preferably selected from the group consisting of phenyl, pyridyl and thienyl, wherein the aryl or heteroaryl residues condensed in this way can for their part be respectively unsubstituted or mono- or polysubstituted.

In case R⁶⁰⁹ and R⁶¹⁰ of the compound of general formula (6-I) form together with the nitrogen atom connecting them a 3 to 10 membered heterocycloaliphatic residue, preferably a 4 to 7 membered heterocycloaliphatic residue, unsubstituted or mono- or polysubstituted, said heterocycloaliphatic residue may optionally be condensed with aryl or heteroaryl or with a C₃₋₁₀ cycloaliphatic residue or with a 3 to 10 membered heterocycloaliphatic residue, wherein the aryl, heteroaryl, C₃₋₁₀ cycloaliphatic or 3 to 10 membered heterocycloaliphatic residues condensed in this way can for their part be respectively unsubstituted or mono- or polysubstituted. Preferably, said heterocycloaliphatic residue formed by R⁶⁰⁹ and R⁶¹⁰ of the compound of general formula (6-1) together with the nitrogen atom connecting them may optionally be condensed with aryl or heteroaryl, wherein the aryl, or heteroaryl, preferably selected from the group consisting of phenyl, pyridyl and thienyl condensed in this way can for their part be respectively unsubstituted or mono- or polysubstituted.

Particularly preferably, in case R⁶⁰⁹ and R⁶¹⁰ form together with the nitrogen atom connecting them a 3 to 10 membered heterocycloaliphatic residue, preferably a 4 to 7 membered heterocycloaliphatic residue, unsubstituted or mono- or polysubstituted, said heterocycloaliphatic residue may optionally be condensed with aryl or heteroaryl, preferably selected from the group consisting of phenyl, pyridyl and thienyl, wherein the aryl or heteroaryl residues condensed in this way can for their part be respectively unsubstituted or mono- or polysubstituted.

Yet another preferred embodiment of present invention is a compound according to general formula (6-I), wherein
R⁶⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, a S(═O)₂—C₁₋₄-aliphatic residue, CF₃, CN, a C₁₋₄-aliphatic residue and C(═O)—OH, preferably denotes a C₁₋₁₀-aliphatic residue, more preferably a C₁₋₈-aliphatic residue, unsubstituted or mono- or polysubstituted with at least one substituent selected from the group consisting of F, CI, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, a S(═O)₂—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

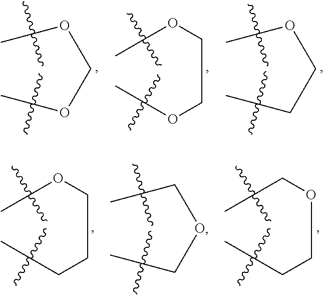 [SMILES: *OCO* ; *OCCO* ; *CCO* ; *CCCO* ; *COC* ; *CCOC*]

benzyl, phenyl, thienyl, pyridyl, furyl, thiazolyl and oxazolyl, preferably in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

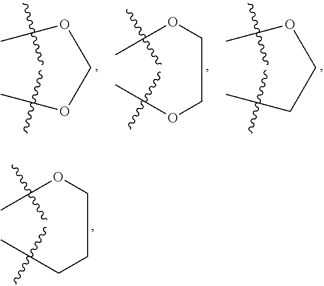 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH,
R⁶⁰² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted,
preferably represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and an O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
R⁶⁰³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue,
   wherein the C₁₋₄ aliphatic residue may be in each case be unsubstituted or mono- or polysubstituted with at least one substituent selected from the group consisting of F, Cl, Br, I, ═O, OH, and an unsubstituted O—C₁₋₄-aliphatic residue;
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
R⁶⁰⁴ denotes a C₁₋₁₀-aliphatic residue, preferably a C₁₋₈-aliphatic residue,
unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R⁶⁰⁴ denotes a 3 to 10 membered heterocycloaliphatic residue, the 3 to 10 membered heterocycloaliphatic residue is linked via a carbon atom,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

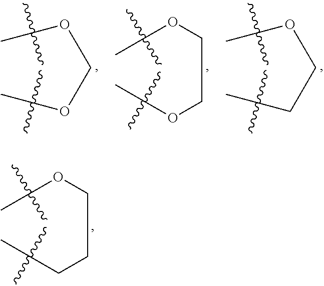 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or poly-substituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged, preferably in each case is bridged, via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH,
R⁶⁰⁵ denotes H or a C₁₋₁₀-aliphatic residue, preferably a C₁₋₄-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or
R⁶⁰⁴ and R⁶⁰⁵ form together with the nitrogen atom connecting them a 3 to 10 membered heterocycloaliphatic residue, preferably a 4 to 7 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, ═O, OCF₃, CF₃ and an unsubstituted O—C₁₋₄-aliphatic residue, preferably selected from the group consisting of F, Cl, Br, I, OH, OCF₃, CF₃ and an unsubstituted O—C₁₋₄-aliphatic residue and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the 3 to 10 membered heterocycloaliphatic residue formed by R⁴ and R⁵ together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue

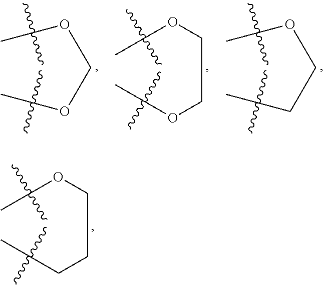 [SMILES: *OCO* ; *OCCO* ; *CCO* ; *CCCO*]

benzyl, phenyl, thienyl, pyridyl, furyl, thiazolyl and oxazolyl, and wherein the 3 to 10 membered heterocycloaliphatic residue formed by R⁴ and R⁵ together with the nitrogen atom connecting them may optionally be condensed with a C₃₋₁₀ cycloaliphatic residue or a 3 to 10 membered heterocycloaliphatic residue, wherein the C₃₋₁₀ cycloaliphatic residue or the 3 to membered heterocycloaliphatic residue condensed in this way can for their part be respectively unsubstituted or mono- or polysubstituted with at least one substituent selected from the group consisting of F, Cl, Br, I, NO₂, NH₂, and NH(C₁₋₄ aliphatic residue), an N(C₁₋₄ aliphatic residue)₂, ═O, OH, an O—C₁₋₄ aliphatic residue, OCF₃, SH, SCF₃, a S—C₁₋₄ aliphatic residue, CF₃, CN, a C₁₋₄ aliphatic residue, C(═O)—OH, C(═O)—CH₃, C(═O)—C₂H₅, C(═)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
R⁶⁰⁶ denotes a C₂₋₁₀-aliphatic residue, preferably a C₂₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
on the condition that if R⁶⁰⁶ denotes a 3 to 10 membered heterocycloaliphatic residue, the 3 to 10 membered heterocycloaliphatic residue is linked via a carbon atom, or
R⁶⁰⁶ denotes S—R⁶⁰⁷, O—R⁶⁰⁸ or N(R⁶⁰⁹R⁶¹⁰),
wherein
   R⁶⁰⁷ and R⁶⁰⁸ in each case represent a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
   or in each case represent a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁶⁰⁷ or R⁶⁰⁸ denotes a 3 to 10 membered heterocycloaliphatic residue, the 3 to 10 membered heterocycloaliphatic residue is linked via a carbon atom of the 3 to 10 membered heterocycloaliphatic residue,
   R⁶⁰⁹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
   on the condition that if R⁶⁰⁹ denotes a 3 to 10 membered heterocycloaliphatic residue, the 3 to 10 membered heterocycloaliphatic residue is linked via a carbon atom of the 3 to 10 membered heterocycloaliphatic residue,
   R⁶¹⁰ denotes H or a C₁₋₁₀-aliphatic residue, preferably a C₁₋₄-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH; preferably denotes a C₁₋₁₀-aliphatic residue, more preferably a C₁₋₄-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or
   R⁶⁰⁹ and R⁶¹⁰ form together with the nitrogen atom connecting them a 3 to 10 membered heterocycloaliphatic residue, preferably a 3 to 6 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the 3 to 10 membered heterocycloaliphatic residue formed by R⁶⁰⁹ and R⁶¹⁰ together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

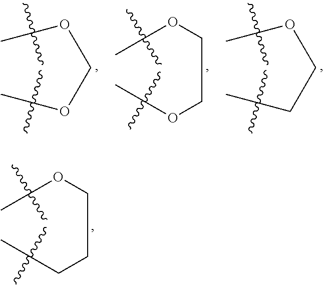 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In a preferred embodiment of the compound according to general formula (6-I), the residue
R⁶⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, a S(═O)₂—C₁₋₄-aliphatic residue, CF₃, CN, a C₁₋₄-aliphatic residue and C(═O)—OH, preferably denotes a C₁₋₁₀-aliphatic residue, more preferably a C₁₋₈-aliphatic residue, unsubstituted or mono- or polysubstituted with at least one substituent selected from the group consisting of F, CI, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or poly-substituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, a S(═O)₂—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

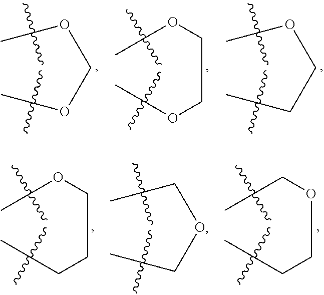 [SMILES: *OCO* ; *OCCO* ; *CCO* ; *CCCO* ; *COC* ; *CCOC*]

benzyl, phenyl, thienyl, pyridyl, furyl, thiazolyl and oxazolyl, preferably in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

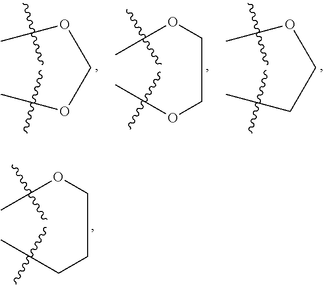 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH.

In a further preferred embodiment of the compound according to general formula (6-I), the residue
R⁶⁰¹ represents the partial structure (6-T1)

—(CR^{612a}R^{612b})ₘ₆—R^{612c} (6-T1),
wherein
m6 denotes 0, 1, 2, 3 or 4, preferably denotes 0, 1, 2 or 3, more preferably denotes 0, 1, or 2,
R^{612a} and R^{612b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, preferably each independently of one another represent H, F, Cl, Br, I, NH₂, a NH(C₁₋₄ aliphatic residue), a N(C₁₋₄ aliphatic residue)₂, OH, O—C₁₋₄ aliphatic residue or a C₁₋₄ aliphatic residue, or together denote ═O,
more preferably each independently of one another represent H, F, Cl, Br, I, OH, an O—C₁₋₄ aliphatic residue or a C₁₋₄ aliphatic residue, or together denote ═O,
even more preferably each independently of one another represent H, F, OH, an O—C₁₋₄ aliphatic residue or a C₁₋₄ aliphatic residue, or together denote ═O, and
R^{612c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, a S(═O)₂—C₁₋₄ aliphatic residue, CF₃, CN, a C₁₋₄-aliphatic residue and C(═O)—OH, preferably denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue, preferably when m6 is 0,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes—preferably when m6 is 0 or 2, more preferably when m6 is 0—an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, a S(═O)₂—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

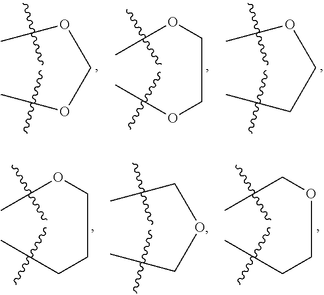 [SMILES: *OCO* ; *OCCO* ; *CCO* ; *CCCO* ; *COC* ; *CCOC*]

benzyl, phenyl, thienyl, pyridyl, furyl, thiazolyl and oxazolyl, preferably denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

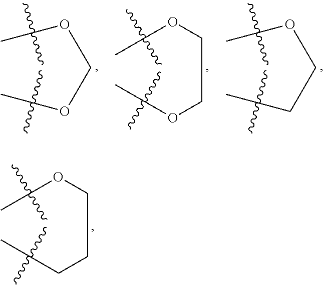 [SMILES: *OCO* ; *OCCO* ; *CCO* ; *CCCO*]

benzyl, phenyl, thienyl, pyridyl, furyl, thiazolyl and oxazolyl, preferably when m6 is ═O,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R⁶⁰¹ represents the partial structure (6-T1),
   wherein
   m6 denotes 0, 1, 2 or 3, preferably denotes 0, 1 or 2,
   R^{612a} and R^{612b} each independently of one another represent H, F, Cl, Br, I, OH, an O—C₁₋₄ aliphatic residue or a C₁₋₄ aliphatic residue, or together denote ═O,
      preferably each independently of one another represent H, F, OH, a O—C₁₋₂ aliphatic residue or a C₁₋₂ aliphatic residue, or together denote ═O, and
   R^{612c} denotes a C₁₋₄ aliphatic residue, unsubstituted or mono- or polysubstituted with at least one substituent selected from the group consisting of F, Cl, Br, I, OH, ═O, an O—C₁₋₄ aliphatic residue, OCF₃, CF₃, CN, a C₁₋₄-aliphatic residue, a S(═O)₂—C₁₋₄-aliphatic residue and C(═O)—OH, preferably
   denotes a C₁₋₄ aliphatic residue, unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes—preferably when m6 is 0 or 2, more preferably when m6 is 0—an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, a S(═O)₂—C₁₋₄ aliphatic residue, NO₂, N(C₁₋₄ aliphatic residue)₂, CF₃, CN, a C₁₋₄-aliphatic residue, C(═O)—OH, C(═O)—CH₃, C(═O)—C₂H₅, C(═O)—O—CH₃, C(═O)—O—C₂H,

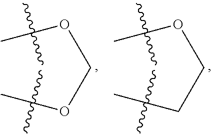 [SMILES: *OCO* ; *CCO*]

a C₃₋₆ cycloaliphatic residue, a 3 to 6 membered heterocycloaliphatic residue, benzyl, phenyl, thienyl, pyridyl, furyl, thiazolyl or oxazolyl, preferably denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃, C(═O)—O—C₂H₅, a C₃₋₆ cycloaliphatic residue, a 3 to 6 membered heterocycloaliphatic residue, benzyl, phenyl, thienyl, pyridyl, furyl, thiazolyl or oxazolyl, preferably when m6 is 0,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

More preferably,
R⁶⁰¹ represents the partial structure (6-T1),
   wherein
   m6 denotes 0, 1, 2 or 3, preferably denotes 0, 1 or 2,
   R^{612a} and R^{612b} each independently of one another represent H, F, Cl, Br, I, OH, an O—C₁₋₄ aliphatic residue or a C₁₋₄ aliphatic residue, or together denote ═O, preferably each independently of one another represent H, F, OH, a O—C₁₋₂ aliphatic residue or a C₁₋₂ aliphatic residue, or together denote ═O, and
   R^{612c} denotes a C₁₋₄ aliphatic residue, unsubstituted or mono- or polysubstituted with at least one substituent selected from the group consisting of F, Cl, Br, I, OH, an O—C₁₋₄ aliphatic residue, CF₃, CN, a S(═O)₂—C₁₋₄-aliphatic residue and a C₁₋₄-aliphatic residue, preferably denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
   or denotes—preferably when m6 is 0 or 2, more preferably when m6 is 0—an aryl or heteroaryl, in each case unsubstituted or mono- or polysubstituted with at least one substituent selected from the group consisting of F, Cl, Br, I, OH, an O—C₁₋₄ aliphatic residue, OCF₂H, OCF₃, CF₃, CN, a C₁₋₄-aliphatic residue, C(═O)—CH₃, C(═O)—C₂H₅, CH₂—OH, CH₂—OCH₃, S(═O)₂—CH₃, SCF₃, NO₂, N(C₁₋₄ aliphatic residue)₂,

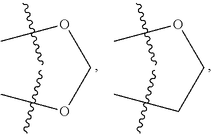 [SMILES: *OCO* ; *CCO*]

C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, preferably denotes—preferably when m6 is 0 or 2, more preferably when m6 is 0—an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl,
   wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In a further preferred embodiment of the compound according to general formula (6-I), the residue
R⁶⁰¹ represents the partial structure (6-T1),
   wherein
   m6 is 0, 1 or 2, preferably 0 or 2, more preferably 2, and
   R^{612a} and R^{612b} each independently of one another represent H, F, OH, a O—C₁₋₄ aliphatic residue or a C₁₋₄ aliphatic residue or together denote ═O; preferably H, F, OH, CH₃ or OCH₃ or together denote ═O;
   R^{612c} denotes a C₁₋₄ aliphatic residue, unsubstituted or mono- or polysubstituted with at least one substituent selected from the group consisting of F, Cl, Br, I, CN, OH, an unsubstituted O—C₁₋₄ aliphatic residue, an unsubstituted S(═O)₂—C₁₋₄ aliphatic residue, CF₃, and an unsubstituted C₁₋₄-aliphatic residue, preferably denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
   wherein
   m6 is 0 or 2, more preferably 0, and
   R^{612a} and R^{612b} each independently of one another represent H, F, OH, a O—C₁₋₄ aliphatic residue or a C₁₋₄ aliphatic residue; preferably H, F, OH, CH₃ or OCH₃; and
   R^{612c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, OCF₂H, CH₂—OH, CH₂—OCH₃, S(═O)₂—CH₃, SCF₃, NO₂, N(C₁₋₄ aliphatic residue)₂,

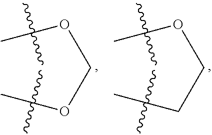 [SMILES: *OCO* ; *CCO*]

CF₃, CN, a C₁₋₄-aliphatic residue, C(═O)—CH₃, C(═O)—C₂H₅, C(═O)—O—CH₃, C(═O)—O—C₂H₅ and phenyl, preferably denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃.

Preferably,
R⁶⁰¹ represents the partial structure (6-T1),
   wherein
   m6 is 0, 1 or 2, preferably 0 or 2, more preferably 2, and
   R^{612a} and R^{612b} each independently of one another represent H, F, OH, CH₃ or OCH₃ or together denote ═O, more preferably H, F, OH or CH₃, even more preferably H,
   R^{612c} denotes a C₁₋₄ aliphatic residue, preferably methyl, ethyl, n-propyl, 2-propyl, n-butyl, isobutyl, sec-butyl, or tert-butyl, unsubstituted or mono- or polysubstituted with at least one substituent selected from the group consisting of F, Cl, Br, I, CN, OH, S(═O)₂—CH₃, an unsubstituted O—C₁₋₄ aliphatic residue, preferably O-methyl and O-tert.-butyl, and CF₃, preferably denotes a C₁₋₄ aliphatic residue, preferably methyl, ethyl, n-propyl, 2-propyl, n-butyl, isobutyl, sec.-butyl, or tert.-butyl, unsubstituted or mono- or polysubstituted with at least one substituent selected from the group consisting of F, Cl, Br, I, an unsubstituted O—C₁₋₄ aliphatic residue, preferably O-methyl and O-tert-butyl, and CF₃,
   or denotes a C₃₋₁₀-cycloaliphatic residue or a 3 to 10 membered heterocycloaliphatic residue, preferably cyclopropyl, cyclopentyl, cyclohexyl, morpholinyl, oxetanyl, or tetrahydropyranyl, in each case unsubstituted or mono- or polysubstituted with at least one substituent selected from the group consisting of F, Cl, Br, I, an unsubstituted O—C₁₋₄ aliphatic residue, preferably O-methyl and O-ethyl, CF₃, and an unsubstituted C₁₋₄-aliphatic residue, preferably methyl or ethyl, or
   wherein
   m6 is 0 or 2, more preferably 0, and
   R^{612a} and R^{612b} each independently of one another represent H, F, OH, CH₃ or OCH₃; preferably H, OH or CH₃, and
   R^{612c} denotes an aryl or heteroaryl, preferably phenyl or pyridyl, in each case unsubstituted or mono- or polysubstituted with at least one substituent selected from the group consisting of F, Cl, Br, I, OH, an O—C₁₋₄ aliphatic residue, preferably OCH₃, OCF₃, OCF₂H, CH₂—OH, CH₂—OCH₃, S(═O)₂—CH₃, SCF₃, NO₂, N(CH₃)₂,

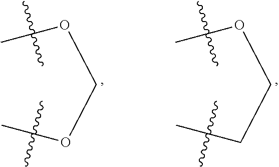 [SMILES: *OCO* ; *CCO*]

CF₃, CN, a C₁₋₄-aliphatic residue, C(═O)—CH₃, C(═O)—C₂H₅, C(═O)—O—CH₃, C(═O)—O—C₂H₅ and phenyl, preferably denotes an aryl or heteroaryl, preferably phenyl or pyridyl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃.

Particularly preferred is a compound according to general formula (I) which has the following general formula (6-I-d),

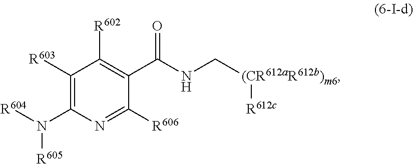

wherein the particular radicals and parameters have the meanings described herein in connection with the compounds according to the invention and preferred embodiments thereof.

In a preferred embodiment of the compound according to general formula (6-I), the residue
R⁶⁰² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted.

Preferably,
R⁶⁰² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

More preferably,
R⁶⁰² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
cyclopropyl, cyclobutyl, cyclopentyl, cyclohexyl, pyrrolidinyl, piperazinyl, 4-methylpiperazinyl, morpholinyl, or piperidinyl, preferably cyclopropyl, cyclobutyl, cyclopentyl or cyclohexyl, in each case unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
   and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted O—C₁₋₄-aliphatic residue.

Even more preferably,
R⁶⁰² represents F; Cl; Br; I; CN; CF₃; NO₂; OCF₃; SCF₃; methyl; ethyl; n-propyl; iso-propyl; n-butyl; sec.-butyl; tert.-butyl; CH₂—OH; CH₂—O—CH₃; CH₂—CH₂—OH; CH₂—CH₂—OCH₃; O-methyl; O-ethyl; O—(CH₂)₂—O—CH₃; O—(CH₂)₂—OH; S-Methyl; S-ethyl; cyclopropyl, cyclobutyl, cyclopentyl, and cyclohexyl; preferably represents F; Cl; Br; I; CN; CF₃; NO₂; OCF₃; SCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; S-ethyl; cyclopropyl, cyclobutyl, cyclopentyl, and cyclohexyl.

Still more preferably,
R⁶⁰² is selected from the group consisting of F; Cl; CF₃; CN; SCF₃; OCF₃; CH₃; C₂H₅; n-propyl; iso-propyl; t-butyl; CH₂—OH; CH₂—O—CH₃; cyclopropyl; O—CH₃ and O—C₂H₅; preferably is selected from the group consisting of F; Cl; CF₃; CN; SCF₃; OCF₃; CH₃; C₂H₅; n-propyl; iso-propyl; t-butyl; cyclopropyl; O—CH₃ and O—C₂H₅.

In particular,
R⁶⁰² is selected from the group consisting of F; Cl; CF₃; CH₃; C₂H₅, iso-propyl; CH₂—O—CH₃; cyclopropyl; and O—CH₃; preferably is selected from the group consisting of F; Cl; CF₃; CH₃; C₂H₅, iso-propyl; cyclopropyl; and O—CH₃.

More particular,
R⁶⁰² is selected from the group consisting of CF₃; CH₃; C₂H₅, iso-propyl; CH₂—O—CH₃; and O—CH₃; preferably is selected from the group consisting of CH₃; C₂H₅, iso-propyl; CH₂—O—CH₃; and O—CH₃.

In a particular preferred embodiment of the compound according to general formula (6-I), the residue
R⁶⁰² denotes CH₃ or CF₃, most preferably R⁶⁰² denotes CH₃.

In a further preferred embodiment of the compound according to general formula (6-I), the residue
R⁶⁰³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue,
   wherein the C₁₋₄ aliphatic residue may be in each case be unsubstituted or mono- or polysubstituted with at least one substituent selected from the group consisting of F, Cl, Br, I, ═O, OH, and an unsubstituted O—C₁₋₄-aliphatic residue;
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

Preferably,
R⁶⁰³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue,
   wherein the C₁₋₄ aliphatic residue may be in each case be unsubstituted or mono- or polysubstituted with at least one substituent selected from the group consisting of F, Cl, Br, I, ═O, OH, and an unsubstituted O—C₁₋₄-aliphatic residue.

More preferably,
R⁶⁰³ represents H; F; Cl; Br; I; CN; CF₃; SCF₃; NO₂; OCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; or S-Ethyl.

Even more preferably
R⁶⁰³ represents H; F; Cl; Br; I; CN; CF₃; SCF₃; OCF₃; methyl; ethyl; O-methyl; or O-ethyl, preferably represents H; F; Cl; Br; I; CF₃; SCF₃; OCF₃; methyl; ethyl; O-methyl; or O-ethyl.

Still more preferably
R⁶⁰³ represents H; F; Cl; Br; CN; CF₃; SCF₃; OCF₃; O-methyl or methyl, preferably represents H; F; Cl; CF₃; SCF₃; OCF₃; O-methyl or methyl.

In particular
R⁶⁰³ represents H; F; Cl; Br; CN; or methyl, preferably H, F, Cl, Br or CN, more preferably H, Cl or Br, most preferably H.

In a further preferred embodiment of the compound according to general formula (6-I), the residue
R⁶⁰⁴ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R⁶⁰⁴ denotes a 3 to 10 membered heterocycloaliphatic residue, the 3 to 10 membered heterocycloaliphatic residue is linked via a carbon atom,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

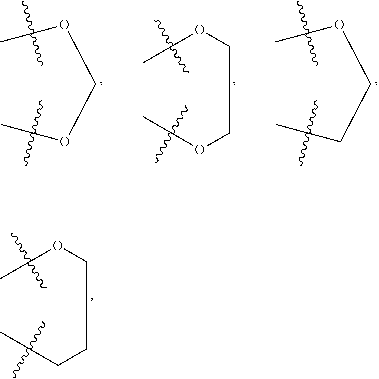 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged, preferably in each case is bridged, via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH,
R⁶⁰⁵ denotes H or a C₁₋₁₀-aliphatic residue, preferably a C₁₋₄-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or
R⁶⁰⁴ and R⁶⁰⁵ form together with the nitrogen atom connecting them a 3 to 10 membered heterocycloaliphatic residue, preferably a 4 to 7 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, ═O, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the 3 to 10 membered heterocycloaliphatic residue formed by R⁴ and R⁵ together with the nitrogen atom connecting them may optionally be condensed with aryl or heteroaryl, preferably with phenyl, pyridyl or thienyl, wherein the aryl or heteroaryl residues condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

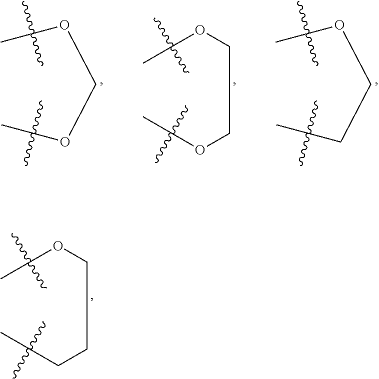 [SMILES: *OCO* ; *OCCO* ; *CCO* ; *CCCO*]

benzyl, phenyl, thienyl, pyridyl, furyl, thiazolyl and oxazolyl,
and wherein the 3 to 10 membered heterocycloaliphatic residue formed by R⁴ and R⁵ together with the nitrogen atom connecting them may optionally be condensed with a C₃₋₁₀ cycloaliphatic residue or a 3 to 10 membered heterocycloaliphatic residue, wherein the C₃₋₁₀ cycloaliphatic residue or a 3 to 10 membered heterocycloaliphatic residue condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, ═O, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In a further preferred embodiment of the compound according to general formula (6-I), the residue
R⁶⁰⁴ represents the partial structure (6-T2)

—(CR^{613a}R^{613b})ₙ₆—R^{613c} (6-T2),
wherein
n6 denotes 0, 1, 2, or 3, preferably denotes 1, 2 or 3, more preferably denotes 1 or 2, even more preferably denotes 1,
R^{613a} and R^{613b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O,
preferably each independently of one another represent H, F, Cl, Br, I, NH₂, a NH(C₁₋₄ aliphatic residue), a N(C₁₋₄ aliphatic residue)₂, OH, O—C₁₋₄ aliphatic residue or a C₁₋₄ aliphatic residue or together denote ═O,
more preferably each independently of one another represent H, F, Cl, Br, I, an O—C₁₋₄ aliphatic residue or a C₁₋₄ aliphatic residue or together denote ═O, even more preferably each independently of one another represent H, F, an O—C₁₋₄ aliphatic residue or a C₁₋₄ aliphatic residue or together denote ═O, and
R^{613c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes—preferably when n6 is ≠0, more preferably when n is 1—a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes—preferably when n6 is ≠0, more preferably when n6 is 1, —an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

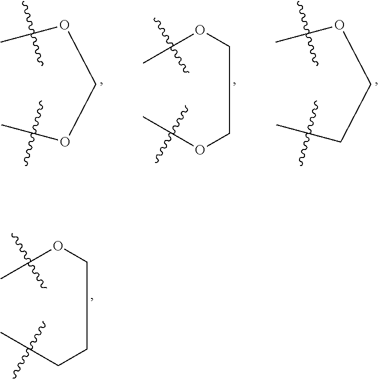 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
R⁶⁰⁵ denotes H or a C₁₋₁₀-aliphatic residue, preferably a C₁₋₆-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or
R⁶⁰⁴ and R⁶⁰⁵ form together with the nitrogen atom connecting them a 3 to 10 membered heterocycloaliphatic residue, preferably a 4 to 7 membered heterocycloaliphatic residue, or preferably selected from the group consisting of morpholinyl, piperidinyl, pyrrolidinyl, azetidinyl, piperazinyl, 4-methylpiperazinyl, oxazepanyl, thiomorpholinyl, azepanyl,

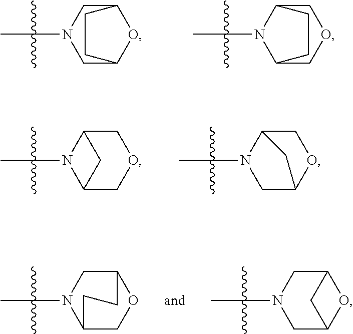

more preferably selected from the group consisting of morpholinyl, piperidinyl, pyrrolidinyl, azetidinyl, piperazinyl, 4-methylpiperazinyl, oxazepanyl, in each case unsubstituted or mono- or polysubstituted with at least one substituent selected from the group consisting of F, Cl, Br, I, NO₂, OH, ═O, C(═O)—OH, an O—C₁₋₄ aliphatic residue, OCF₃, SH, SCF₃, a S—C₁₋₄ aliphatic residue, CF₃, CN, and a C₁₋₄-aliphatic residue, a C₃₋₆ cycloaliphatic residue, preferably cyclopropyl, cyclobutyl or cyclopentyl, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, ═O, OH, CF₃ and an unsubstituted O—C₁₋₄-aliphatic residue, preferably selected from the group consisting of F, Cl, Br, I, OH, CF₃ and an unsubstituted O—C₁₋₄-aliphatic residue,
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the 3 to 10 membered heterocycloaliphatic residue formed by R⁴ and R⁵ together with the nitrogen atom connecting them may optionally be condensed with aryl or heteroaryl, preferably with phenyl, pyridyl or thienyl,
wherein the aryl or heteroaryl residues condensed in this way can for their part be respectively unsubstituted or mono- or polysubstituted with at least one substituent selected from the group consisting of F, Cl, Br, I, NO₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, a 3 to 6 membered heterocycloaliphatic residue,

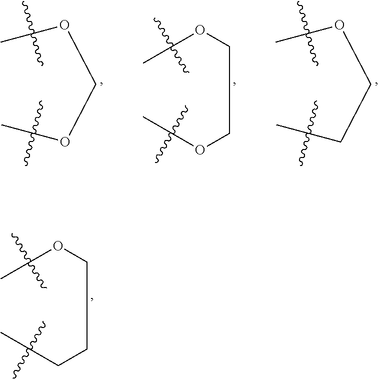 [SMILES: *OCO* ; *OCCO* ; *CCO* ; *CCCO*]

benzyl, phenyl, thienyl, and pyridyl,
and wherein the 3 to 10 membered heterocycloaliphatic residue formed by R⁴ and R⁵ together with the nitrogen atom connecting them may optionally be condensed with a C₃₋₁₀ cycloaliphatic residue, preferably cyclopropyl, cyclobutyl or cyclopentyl, or a 3 to 10 membered heterocycloaliphatic residue, preferably oxetanyl or oxiranyl, wherein the C₂₋₁₀ cycloaliphatic residue or the 3 to 10 membered heterocycloaliphatic residue condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, ═O, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, and pyridyl, may in each case may be unsubstituted or mono- or polysubstituted with at least one substituent selected from the group consisting of F, Cl, Br, I, NO₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, and C(═O)—OH, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R⁶⁰⁴ represents the partial structure (6-T2),
   wherein
   n6 denotes 0, 1, 2, or 3, preferably denotes 1, 2 or 3, more preferably denotes 1 or 2, even more preferably denotes 1,
   R^{613a} and R^{613b} each independently of one another represent H, F, Cl, Br, I, an O—C₁₋₄ aliphatic residue or a C₁₋₄ aliphatic residue or together denote ═O,
   preferably each independently of one another represent H, F, a O—C₁₋₂ aliphatic residue or a C₁₋₂ aliphatic residue or together denote ═O, and
   R^{613c} denotes a C₁₋₄ aliphatic residue, unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH, or denotes—preferably when n6 is ≠0, more preferably when n6 is 1—a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes—preferably when n6 is ≠0, more preferably when n6 is 1—an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl or oxazolyl,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein benzyl, phenyl, thienyl, pyridyl, furyl, thiazolyl and oxazolyl may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH,
R⁶⁰⁵ denotes H or a C₁₋₆-aliphatic residue, preferably a C₁₋₄-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or
R⁶⁰⁴ and R⁶⁰⁵ form together with the nitrogen atom connecting them a 3 to 10 membered heterocycloaliphatic residue, preferably a 4 to 7 membered heterocycloaliphatic residue, or preferably selected from the group consisting of morpholinyl, piperidinyl, pyrrolidinyl, azetidinyl, piperazinyl, 4-methylpiperazinyl, oxazepanyl, thiomorpholinyl, azepanyl,

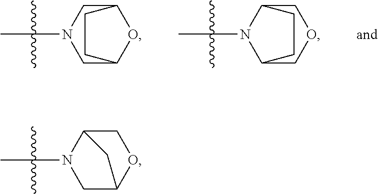 [SMILES: *N1CC2CCC(C1)O2 ; *N1C2CCC1COC2 ; "and" ; *N1CC2CC1CO2]

more preferably selected from the group consisting of morpholinyl, piperidinyl, pyrrolidinyl, azetidinyl, piperazinyl, 4-methylpiperazinyl, oxazepanyl, unsubstituted or mono- or polysubstituted with at least one substituent selected from the group consisting of F, Cl, Br, I, OH, ═O, C(═O)—OH, an O—C₁₋₄ aliphatic residue, OCF₃, SCF₃, a S—C₁₋₄ aliphatic residue, CF₃, and a C₁₋₄-aliphatic residue, and a C₃₋₆ cycloaliphatic residue, preferably cyclopropyl, cyclobutyl or cyclopentyl,
   wherein the C₁₋₄-aliphatic residue in each case may be unsubstituted or mono- or polysubstituted with at least one substituent selected from the group consisting of F, Cl, Br, I, OH, ═O, CF₃ and an unsubstituted O—C₁₋₄-aliphatic residue, preferably selected from the group consisting of F, Cl, Br, I, OH, CF₃ and an unsubstituted O—C₁₋₄-aliphatic residue,
   wherein the C₃₋₆ cycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, CF₃, CN, a C₁₋₄-aliphatic residue and C(═O)—OH,
and wherein the 3 to 10 membered heterocycloaliphatic residue formed by R⁶⁰⁴ and R⁶⁰⁵ together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, Br, I, OH, an O—C₁₋₄ aliphatic residue, OCF₃, SCF₃, a S—C₁₋₄ aliphatic residue, CF₃, a C₁₋₄-aliphatic residue, C(═O)—OH, a C₃₋₆ cycloaliphatic residue, benzyl, phenyl, thienyl, and pyridyl,
and wherein the 3 to 10 membered heterocycloaliphatic residue formed by R⁶⁰⁴ and R⁶⁰⁵ together with the nitrogen atom connecting them may optionally be condensed with a C₃₋₁₀ cycloaliphatic residue, preferably cyclopropyl, cyclobutyl or cyclopentyl, or a 3 to 10 membered heterocycloaliphatic residue, preferably oxetanyl or oxiranyl,
   wherein the C₃₋₁₀ cycloaliphatic residue or the 3 to 10 membered heterocycloaliphatic residue condensed in this way can for their part be respectively unsubstituted or mono- or polysubstituted with at least one substituent selected from the group consisting of F, Cl, Br, I, ═O, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, and pyridyl, may in each case may be unsubstituted or mono- or polysubstituted with at least one substituent selected from the group consisting of F, Cl, Br, I, OH, an O—C₁₋₄ aliphatic residue, OCF₃, O—CH₂—OH, O—CH₂—O—CH₃, SCF₃, a S—C₁₋₄ aliphatic residue, CF₃, CN, a C₁₋₄-aliphatic residue, and C(═O)—OH, and
   wherein the C₃₋₆ cycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, CF₃, a C₁₋₄-aliphatic residue and C(═O)—OH.

More preferably,
R⁶⁰⁴ represents the partial structure (6-T2),
   wherein
   n6 denotes 0, 1, 2 or 3, preferably denotes 1 or 2, more preferably denotes 1,
   R^{613a} and R^{613b} each independently of one another represent H, F, Cl, Br, I, an O—C₁₋₄ aliphatic residue or a C₁₋₄ aliphatic residue or together denote ═O,
   preferably each independently of one another represent H, F, a O—C₁₋₂ aliphatic residue or a C₁₋₂ aliphatic residue or together denote ═O, and
   R^{613c} denotes a C₁₋₄ aliphatic residue, unsubstituted or mono- or polysubstituted with at least one substituent selected from the group consisting of F, Cl, Br, I, ═O, an O—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
   or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl,
      wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH,
R⁶⁰⁵ denotes H or an unsubstituted C₁₋₄-aliphatic residue or a C₁₋₄-aliphatic residue monosubstituted with O-methyl, wherein the C₁₋₄-aliphatic residue is in each case preferably selected from the group consisting of methyl, ethyl, n-propyl, 2-propyl, n-butyl, isobutyl, sec.-butyl and tert.-butyl, more preferably selected from the group consisting of methyl and ethyl, or
R⁶⁰⁴ and R⁶⁰⁵ form together with the nitrogen atom connecting them a 3 to 10 membered heterocycloaliphatic residue, more preferably selected from the group consisting of morpholinyl, piperidinyl, pyrrolidinyl, azetidinyl, piperazinyl, 4-methylpiperazinyl, oxazepanyl, thiomorpholinyl, azepanyl,

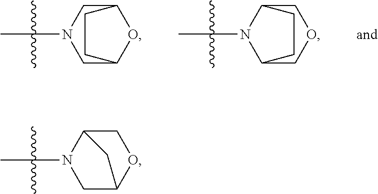 [SMILES: *N1CC2CCC(C1)O2 ; *N1C2CCC1COC2 ; "and" ; *N1CC2CC1CO2]

in each case unsubstituted or mono- or polysubstituted with at least one substituent selected from the group consisting of F, Cl, Br, I, OH, ═O, C(═O)—OH, an O—C₁₋₄ aliphatic residue, OCF₃, SCF₃, a S—C₁₋₄ aliphatic residue, CF₃, and a C₁₋₄-aliphatic residue, cyclopropyl, cyclobutyl and cyclopentyl,
   wherein the C₁₋₄-aliphatic residue is in each case unsubstituted or mono- or polysubstituted with at least one substituent selected from the group consisting of F, Cl, OH, ═O, CF₃ and an unsubstituted O—C₁₋₄-aliphatic residue, preferably is in each case unsubstituted,
and wherein the 3 to 10 membered heterocycloaliphatic residue formed by R⁶⁰⁴ and R⁶⁰⁵ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, Br, I, OH, an O—C₁₋₄ aliphatic residue, OCF₃, SCF₃, a S—C₁₋₄ aliphatic residue, CF₃, a C₁₋₄-aliphatic residue, C(═O)—OH, and a C₃₋₆ cycloaliphatic residue,
   wherein the C₁₋₄-aliphatic residue in each case may be unsubstituted or mono- or polysubstituted with at least one substituent selected from the group consisting of F, Cl, OH, OCF₃, CF₃ and an unsubstituted O—C₁₋₄-aliphatic residue, and
   wherein the C₃₋₆ cycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, CF₃, a C₁₋₄-aliphatic residue and C(═O)—OH,
and wherein the 3 to 10 membered heterocycloaliphatic residue formed by R⁶⁰⁴ and R⁶⁰⁵ together with the nitrogen atom connecting them may optionally be condensed with a C₃₋₆ cycloaliphatic residue, preferably cyclopropyl, cyclobutyl or cyclopentyl, or a 4 to 7 membered heterocycloaliphatic residue, preferably oxetanyl or oxiranyl, wherein the C₃₋₆ cycloaliphatic residue or the 4 to 7 membered heterocycloaliphatic residue condensed in this way can for their part be respectively unsubstituted or mono- or polysubstituted with at least one substituent selected from the group consisting of F, Cl, Br, I, ═O, OH, an O—C₁₋₄ aliphatic residue, OCF₃, SCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅.

Even more preferably,
R⁶⁰⁴ represents the partial structure (I-T2),
   wherein
   n6 denotes 0, 1, 2 or 3, preferably denotes 1 or 2, more preferably denotes 1,
   R^{613a} and R^{613b} each independently of one another represent H, F, a O—C₁₋₄ aliphatic residue or a C₁₋₄ aliphatic residue or together denote ═O; preferably each independently of one another represent H, F, CH₃ or OCH₃ or together denote ═O;
   R^{613c} denotes a C₁₋₄ aliphatic residue, unsubstituted or mono- or polysubstituted with at least one substituent selected from the group consisting of F, Cl, Br, I, ═O, an unsubstituted O—C₁₋₄ aliphatic residue, CF₃, and an unsubstituted C₁₋₄-aliphatic residue,
   or denotes a C₃₋₁₀-cycloaliphatic residue, preferably selected from the group consisting of cyclopropyl, cyclobutyl, cyclopentyl and cyclohexyl, or a 3 to 10 membered heterocycloaliphatic residue, preferably selected from the group consisting of pyrrolidinyl, morpholinyl, piperazinyl, piperidinyl and tetrahydropyranyl, more preferably tetrahydropyranyl, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
   or denotes an aryl or heteroaryl, preferably phenyl or pyridyl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
      wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
R⁶⁰⁵ denotes H or an unsubstituted C₁₋₄-aliphatic residue or a C₁₋₄-aliphatic residue, which is monosubstituted with OCH₃, preferably H, methyl, ethyl, n-propyl, 2-propyl, n-butyl, isobutyl, sec.-butyl or tert.-butyl or CH₂—OCH₃, C₂H₄—OCH₃ or C₃H₆—OCH₃, more preferably H, methyl or ethyl, preferably denotes H or an unsubstituted C₁₋₄-aliphatic residue, preferably H, methyl, ethyl, n-propyl, 2-propyl, n-butyl, isobutyl, sec.-butyl or tert.-butyl, more preferably H, methyl or ethyl, or
R⁶⁰⁴ and R⁶⁰⁵ form together with the nitrogen atom connecting them a morpholinyl, piperidinyl, pyrrolidinyl, azetidinyl, piperazinyl, 4-methylpiperazinyl, oxazepanyl, thiomorpholinyl, azepanyl,

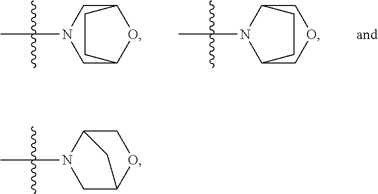 [SMILES: *N1CC2CCC(C1)O2 ; *N1C2CCC1COC2 ; "and" ; *N1CC2CC1CO2]

tetrahydroquinolinyl, tetrahydroisoquinolinyl, tetrahydroimidazo[1,2-a]pyrazinyl, octahydropyrrolo[1,2-a]pyrazinyl,

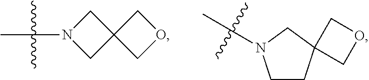 [SMILES: *N1CC2(COC2)C1 ; *N1CCC2(COC2)C1]

dihydroindolinyl, or dihydroisoindolyl, preferably a morpholinyl, piperidinyl, pyrrolidinyl, azetidinyl, piperazinyl, 4-methylpiperazinyl, oxazepanyl, tetrahydroquinolinyl, tetrahydroisoquinolinyl, dihydroindolinyl, or dihydroisoindolyl, in each case unsubstituted or mono- or polysubstituted with at least one substituent selected from the group consisting of F, Cl, Br, I, OH, ═O, C(═O)—OH, an O—C₁₋₄ aliphatic residue, OCF₃, SCF₃, a S—C₁₋₄ aliphatic residue, CF₃, and a C₁₋₄-aliphatic residue, cyclopropyl, cyclobutyl and cyclopentyl,
   wherein the C₁₋₄-aliphatic residue is in each case unsubstituted or mono- or polysubstituted with at least one substituent selected from the group consisting of F, OH, ═O, CF₃ and an unsubstituted O—C₁₋₄-aliphatic residue, preferably is in each case unsubstituted.

Still more preferably,
R⁶⁰⁴ represents the partial structure (6-T2),
   wherein
   n6 denotes 0, 1, 2 or 3, preferably denotes 1 or 2, more preferably denotes 1,
   R^{613a} and R^{613b} each independently of one another represent H, F, CH₃ or OCH₃ or together denote ═O, preferably each independently of one another represent H or CH₃, more preferably H,
   R^{613c} denotes a C₁₋₄ aliphatic residue, preferably methyl, ethyl, n-propyl, 2-propyl, n-butyl, isobutyl, sec-butyl, or tert-butyl, unsubstituted or mono- or polysubstituted with at least one substituent selected from the group consisting of F, Cl, Br, I, ═O, an unsubstituted O—C₁₋₄ aliphatic residue, and CF₃,
   or denotes cyclopropyl, cyclobutyl, cyclopentyl or cyclohexyl, pyrrolidinyl, morpholinyl, piperazinyl, piperidinyl and tetrahydropyranyl, more preferably tetrahydropyranyl or morpholinyl, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
   or denotes an aryl or heteroaryl, preferably phenyl or pyridyl, more preferably phenyl, in each case unsubstituted or mono- or polysubstituted with at least one substituent selected from the group consisting of F, Cl, Br, I, OH, an O—C₁₋₄ aliphatic residue, OCF₃, CF₃, CN, and a C₁₋₄-aliphatic residue,
R⁶⁰⁵ denotes H, methyl or ethyl or C₂H₄OCH₃ or C₃H₆OCH₃, more preferably H or methyl or ethyl, even more preferably methyl, or
R⁶⁰⁴ and R⁶⁰⁵ form together with the nitrogen atom connecting them a morpholinyl, piperidinyl, pyrrolidinyl, azetidinyl, oxazepanyl, tetrahydroquinolinyl, tetrahydroisoquinolinyl, thiomorpholinyl, azepanyl,

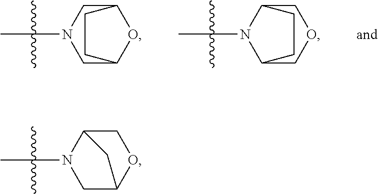 [SMILES: *N1CC2CCC(C1)O2 ; *N1C2CCC1COC2 ; "and" ; *N1CC2CC1CO2]

tetrahydroimidazo[1,2-a]pyrazinyl, octahydropyrrolo[1,2-a]pyrazinyl,

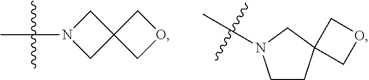 [SMILES: *N1CC2(COC2)C1 ; *N1CCC2(COC2)C1]

dihydroindolinyl, or dihydroisoindolyl, preferably a morpholinyl, piperidinyl, pyrrolidinyl, azetidinyl, oxazepanyl, tetrahydroquinolinyl, tetrahydroisoquinolinyl, dihydroindolinyl, or dihydroisoindolyl, more preferably a morpholinyl, piperidinyl, pyrrolidinyl, azetidinyl, oxazepanyl, tetrahydroquinolinyl, tetrahydroisoquinolinyl, more preferably a morpholinyl, oxazepanyl, tetrahydroquinolinyl, or tetrahydroisoquinolinyl, in each case unsubstituted or mono- or polysubstituted with at least one substituent selected from the group consisting of F, Cl, OH, ═O, C(═O)—OH, C(═O)—CH₃, C(═O)—OCH₃, O-methyl, O-ethyl, OCF₃, SCF₃, CF₃, methyl, CH₂CF₃, CH₂OH, CH₂—OCH₃, CH₂CH₂—OCH₃, ethyl, n-propyl, 2-propyl, cyclopropyl, and cyclobutyl, preferably selected from the group consisting of F, Cl, OH, ═O, C(═O)—OH, O-methyl, O-ethyl, OCF₃, SCF₃, CF₃, methyl, ethyl, n-propyl, 2-propyl, cyclopropyl, and cyclobutyl.

In a preferred embodiment of the compound according to general formula (6-I), the residue
R⁶⁰⁶ denotes a C₂₋₁₀-aliphatic residue, preferably a C₂₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
on the condition that if R⁶⁰⁶ denotes a 3 to 10 membered heterocycloaliphatic residue, the binding is carried out via a carbon atom of the 3 to 10 membered heterocycloaliphatic residue, or
R⁶⁰⁶ denotes S—R⁶⁰⁷, O—R⁶⁰⁸ or N(R⁶⁰⁹R⁶¹⁰),
   wherein
   R⁶⁰⁷ and R⁶⁰⁸ in each case represent a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
   or in each case represent a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   on the condition that if R⁶⁰⁷ or R⁶⁰⁸ denotes a 3 to 10 membered heterocycloaliphatic residue, the binding is carried out via a carbon atom of the 3 to 10 membered heterocycloaliphatic residue,
   R⁶⁰⁹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
   on the condition that if R⁶⁰⁹ denotes a 3 to 10 membered heterocycloaliphatic residue, the binding is carried out via a carbon atom of the 3 to 10 membered heterocycloaliphatic residue,
   R⁶¹⁰ denotes H or a C₁₋₁₀-aliphatic residue, preferably a C₁₋₄-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, preferably denotes a C₁₋₁₀-aliphatic residue, more preferably a C₁₋₄-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or
   R⁶⁰⁹ and R⁶¹⁰ form together with the nitrogen atom connecting them a 3 to 10 membered heterocycloaliphatic residue, preferably a 3 to 6 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   and wherein the 3 to 10 membered heterocycloaliphatic residue formed by R⁶⁰⁹ and R⁶¹⁰ together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

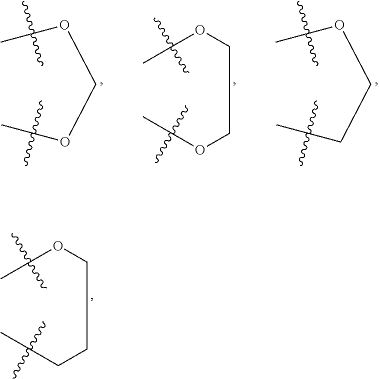 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R⁶⁰⁶ denotes a C₂₋₁₀-aliphatic residue, preferably a C₂₋₈-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue.
on the condition that if R⁶⁰⁶ denotes a 3 to 10 membered heterocycloaliphatic residue, the 3 to 10 membered heterocycloaliphatic residue is linked via a carbon atom, or
   R⁶⁰⁶ denotes S—R⁶⁰⁷ or O—R⁶⁰⁸,
   wherein
   R⁶⁰⁷ and R⁶⁰⁸ in each case represent a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   or in each case denote a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue.
   on the condition that if R⁶⁰⁷ or R⁶⁰⁸ denotes a 3 to 10 membered heterocycloaliphatic residue, the 3 to 10 membered heterocycloaliphatic residue is linked via a carbon atom, or
R⁶⁰⁶ denotes N(R⁶⁰⁹R⁶¹⁰),
   wherein
   R⁶⁰⁹ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
   on the condition that if R⁶⁰⁹ denotes a 3 to 10 membered heterocycloaliphatic residue, the 3 to 10 membered heterocycloaliphatic residue is linked via a carbon atom, and
   R⁶¹⁰ denotes H or a C₁₋₁₀-aliphatic residue, preferably a C₁₋₆-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, preferably denotes a C₁₋₁₀-aliphatic residue, more preferably a C₁₋₆-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or
   R⁶⁰⁹ and R⁶¹⁰ form together with the nitrogen atom connecting them a 3 to 10 membered heterocycloaliphatic residue, preferably a 3 to 6 membered heterocycloaliphatic residue, more preferably selected from the group consisting of morpholinyl, piperidinyl, pyrrolidinyl, azetidinyl and piperazinyl, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   and wherein the 3 to 10 membered heterocycloaliphatic residue formed by R⁶⁰⁹ and R⁶¹⁰ together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, Br, I, NO₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, a 3 to 6 membered heterocycloaliphatic residue,

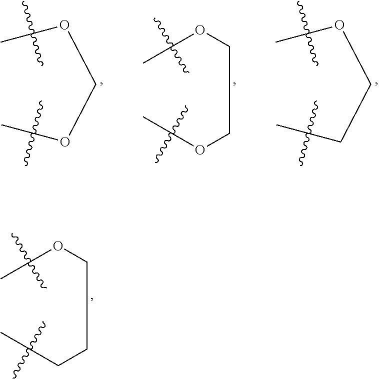 [SMILES: *OCO* ; *OCCO* ; *CCO* ; *CCCO*]

benzyl, phenyl, thienyl, and pyridyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, and pyridyl, may in each case may be unsubstituted or mono- or polysubstituted with at least one substituent selected from the group consisting of F, Cl, Br, I, NO₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, and C(═O)—OH, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

More preferably,
R⁶⁰⁶ denotes a C₂₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
   on the condition that if R⁶⁰⁶ denotes a 3 to 10 membered heterocycloaliphatic residue, the 3 to 10 membered heterocycloaliphatic residue is linked via a carbon atom, or
R⁶⁰⁶ denotes S—R⁶⁰⁷ or O—R⁶⁰⁸,
   wherein
   R⁶⁰⁷ and R⁶⁰⁸ in each case denote a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, CF₃, a C(═O)—O—C₁₋₄-aliphatic residue, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   or in each case denote a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₁₀-cycloaliphatic residue or the 3 to 10 membered heterocycloaliphatic residue may be bridged, preferably is bridged, via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
   on the condition that if R⁶⁰⁷ or R⁶⁰⁸ denotes a 3 to 10 membered heterocycloaliphatic residue, the 3 to 10 membered heterocycloaliphatic residue is linked via a carbon atom, or
R⁶⁰⁶ denotes N(R⁶⁰⁹R⁶¹⁰),
   wherein
   R⁶⁰⁹ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, CF₃, a C(═O)—O—C₁₋₄-aliphatic residue, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₁₀-cycloaliphatic residue or the 3 to 10 membered heterocycloaliphatic residue may in each case be bridged, preferably is bridged, via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue,
   on the condition that if R⁶⁰⁹ denotes a 3 to 10 membered heterocycloaliphatic residue, the 3 to 10 membered heterocycloaliphatic residue is linked via a carbon atom, and
   R⁶¹⁰ denotes H or a C₁₋₆-aliphatic residue, preferably a C₁₋₄-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, preferably denotes a C₁₋₆-aliphatic residue, more preferably a C₁₋₄-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or
   R⁶⁰⁹ and R⁶¹⁰ form together with the nitrogen atom connecting them a 3 to 10 membered heterocycloaliphatic residue, preferably selected from the group consisting of morpholinyl, piperidinyl, pyrrolidinyl, azetidinyl and piperazinyl, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   and wherein the 3 to 10 membered heterocycloaliphatic residue formed by R⁶⁰⁹
   and R⁶¹⁰ together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, Br, I, NO₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, residue,

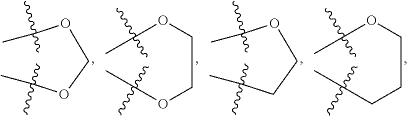 [SMILES: *OCO* ; *OCCO* ; *CCO* ; *CCCO*]

benzyl, phenyl, thienyl, and pyridyl,
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, and
   wherein benzyl, phenyl, thienyl, and pyridyl, may in each case may be unsubstituted or mono- or polysubstituted with at least one substituent selected from the group consisting of F, Cl, Br, I, NO₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, and C(═O)—OH.

Even more preferably,
R⁶⁰⁶ denotes a C₂₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue
   wherein the C₁₋₄-aliphatic residue in each case is unsubstituted,
or denotes a C₃₋₁₀-cycloaliphatic residue, preferably a C₃₋₆-cycloaliphatic residue, or a 3 to 10 membered heterocycloaliphatic residue, preferably a 3 to 6 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with OH or an unsubstituted O—C₁₋₄-aliphatic residue,
and wherein the C₃₋₁₀-cycloaliphatic residue or the 3 to 10 membered heterocycloaliphatic residue may in each case optionally bridged via a unsubstituted C₁₋₄ aliphatic group,
on the condition that if R⁶⁰⁶ denotes a 3 to 10 membered heterocycloaliphatic residue, the 3 to 10 membered heterocycloaliphatic residue is linked via a carbon atom, or
R⁶⁰⁶ denotes S—R⁶⁰⁷ or O—R⁶⁰⁸,
   wherein
   R⁶⁰⁷ and R⁶⁰⁸ in each case denote a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   or in each case denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₁₀-cycloaliphatic residue or the 3 to 10 membered heterocycloaliphatic residue in each case may be bridged, preferably is bridged, via an unsubstituted C₁₋₈ aliphatic group, preferably an unsubstituted C₁₋₄ aliphatic group,
   on the condition that if R⁶⁰⁷ or R⁶⁰⁸ denotes a 3 to 10 membered heterocycloaliphatic residue, the 3 to 10 membered heterocycloaliphatic residue is linked via a carbon atom, or
R⁶⁰⁶ denotes N(R⁶⁰⁹R⁶¹⁰),
   wherein
   R⁶⁰⁹ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₁₀-cycloaliphatic residue or the 3 to 10 membered heterocycloaliphatic residue is in each case bridged via a unsubstituted C₁₋₈ aliphatic group, preferably an unsubstituted C₁₋₄ aliphatic group,
   on the condition that if R⁶⁰⁹ denotes a 3 to 10 membered heterocycloaliphatic residue, the 3 to 10 membered heterocycloaliphatic residue is linked via a carbon atom, and
   R⁶¹⁰ denotes H or an unsubstituted C₁₋₄-aliphatic residue, preferably selected from the group consisting of methyl, ethyl, n-propyl, 2-propyl, n-butyl, isobutyl, sec.-butyl and tert.-butyl, more preferably selected from the group consisting of methyl and ethyl, preferably denotes an unsubstituted C₁₋₄-aliphatic residue, preferably selected from the group consisting of methyl, ethyl, n-propyl, 2-propyl, n-butyl, isobutyl, sec.-butyl and tert.-butyl, more preferably selected from the group consisting of methyl and ethyl, or
   R⁶⁰⁹ and R⁶¹⁰ form together with the nitrogen atom connecting them a 3 to 6 membered heterocycloaliphatic residue, preferably selected from the group consisting of morpholinyl, piperidinyl, pyrrolidinyl, and azetidinyl, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
      wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
   and wherein the 3 to 6 membered heterocycloaliphatic residue formed by R⁶⁰⁹ and R⁶¹⁰ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, residue, benzyl, phenyl, and pyridyl,
      wherein the C₁₋₄-aliphatic residue in each case may be unsubstituted or mono- or polysubstituted with at least one substituent selected from the group consisting of F, Cl, OH, and an unsubstituted O—C₁₋₄-aliphatic residue, and
      wherein benzyl, phenyl, and pyridyl, may in each case may be unsubstituted or mono- or polysubstituted with at least one substituent selected from the group consisting of F, Cl, Br, I, OCH₃, OCF₃, O—CH₂—OH, O—CH₂—O—CH₃, SH, SCF₃, CF₃, and a C₁₋₄-aliphatic residue.

Still more preferably,
R⁶⁰⁶ denotes a C₂₋₆-aliphatic residue, preferably selected from the group consisting of ethyl, n-propyl, 2-propyl, n-butyl, isobutyl, sec.-butyl, tert.-butyl, n-pentyl, isopentyl, neopentyl, n-hexyl, ethenyl and propenyl (—CH₂CH═CH₂, —CH═CH—CH₃, —C(═CH₂)—CH₃), unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue, preferably in each case unsubstituted or mono- or polysubstituted with at least one substituent selected from the group consisting of F, Cl, OH, an O—C₁₋₄-aliphatic residue, CF₃, and a C₁₋₄-aliphatic residue, more preferably in each case unsubstituted or mono- or polysubstituted with at least one substituent selected from the group consisting of F, Cl, and an O—C₁₋₄-aliphatic residue, preferably O-methyl, even more preferably in each case unsubstituted,
   wherein the C₁₋₄-aliphatic residue in each case is unsubstituted,
or denotes a C₃₋₆-cycloaliphatic residue, preferably selected from the group consisting of cyclopropyl, cyclobutyl, cyclopentyl and cyclohexyl, or a 3 to 6 membered heterocycloaliphatic residue, preferably selected from the group consisting of piperidinyl (preferably piperidin-4-yl or piperidin-3-yl), tetrahydrofuranyl, and tetrahydropyranyl, in each case unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue, preferably in each case unsubstituted or mono- or polysubstituted with at least one substituent selected from the group consisting of F, Cl, OH, an O—C₁₋₄-aliphatic residue, CF₃, and a C₁₋₄-aliphatic residue, more preferably in each case unsubstituted or mono- or polysubstituted with at least one substituent selected from the group consisting of F, Cl, and an O—C₁₋₄-aliphatic residue, preferably O-methyl, even more preferably in each case unsubstituted,
   wherein the C₁₋₄-aliphatic residue in each case is unsubstituted,
and wherein the C₃₋₆-cycloaliphatic residue or the 3 to 6 membered heterocycloaliphatic residue may in each case optionally bridged via an unsubstituted C₁₋₄ aliphatic group, preferably via an unsubstituted C₁₋₂ aliphatic group,
on the condition that if R⁶⁰⁶ a 3 to 6 membered heterocycloaliphatic residue, the 3 to 6 membered heterocycloaliphatic residue is linked via a carbon atom, or
R⁶⁰⁶ denotes S—R⁶⁰⁷ or O—R⁶⁰⁸,
   wherein
   R⁶⁰⁷ and R⁶⁰⁸ in each case denote a C₁₋₆-aliphatic residue, preferably selected from the group consisting of methyl, ethyl, n-propyl, 2-propyl, n-butyl, isobutyl, sec-butyl, tert-butyl, n-pentyl, isopentyl, neopentyl, n-hexyl, ethenyl and propenyl (—CH₂CH═CH₂, —CH═CH—CH₃, —C(═CH₂)—CH₃), unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, CF₃, and a C₁₋₄-aliphatic residue, more preferably in each case unsubstituted or mono- or polysubstituted with at least one substituent selected from the group consisting of F, Cl, OH, and an O—C₁₋₄-aliphatic residue,
      wherein the C₁₋₄-aliphatic residue in each case is unsubstituted,
   or denotes a C₃₋₆-cycloaliphatic residue, preferably cyclopropyl, or a 3 to 6 membered heterocycloaliphatic residue, preferably oxetanyl, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, preferably in each case unsubstituted or mono- or polysubstituted with at least one substituent selected from the group consisting of F, Cl, OH, an O—C₁₋₄-aliphatic residue, CF₃, and a C₁₋₄-aliphatic residue,
      wherein the C₁₋₄-aliphatic residue in each case may be unsubstituted or mono- or polysubstituted with OH or an unsubstituted O—C₁₋₄-aliphatic residue,
   and wherein the C₃₋₁₀-cycloaliphatic residue or the 3 to 10 membered heterocycloaliphatic residue in each case may be bridged, preferably is bridged, via an unsubstituted C₁₋₄ aliphatic group,
   on the condition that if R⁶⁰⁷ or R⁶⁰⁸ denotes a 3 to 10 membered heterocycloaliphatic residue, the 3 to 10 membered heterocycloaliphatic residue is linked via a carbon atom, or
R⁶⁰⁶ denotes N(R⁶⁰⁹R⁶¹⁰),
   wherein
   R⁶⁰⁹ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue, preferably an unsubstituted C₁₋₆-aliphatic residue, more preferably selected from the group consisting of methyl, ethyl, n-propyl, 2-propyl, n-butyl, isobutyl, sec.-butyl, tert.-butyl, n-pentyl, isopentyl, neopentyl, and n-hexyl,
      wherein the C₁₋₄-aliphatic residue in each case is unsubstituted,
   or denotes a C₃₋₆-cycloaliphatic residue, preferably selected from the group consisting of cyclopropyl, cyclobutyl, cyclopentyl and cyclohexyl, or a 3 to 6 membered heterocycloaliphatic residue, preferably selected from the group consisting of piperidinyl (preferably piperidin-4-yl or piperidin-3-yl), tetrahydrofuranyl, and tetrahydropyranyl, in each case unsubstituted or mono- or polysubstituted with at least one substituent selected from the group consisting of F, Cl, a C₁₋₄-aliphatic residue and an O—C₁₋₄-aliphatic residue, even more preferably in each case unsubstituted,
      wherein the C₁₋₄-aliphatic residue in each case is unsubstituted,
   and wherein the C₃₋₆-cycloaliphatic residue or the 3 to 6 membered heterocycloaliphatic residue may in each case optionally bridged via an unsubstituted C₁₋₄ aliphatic group, on the condition that if R⁶⁰⁹ denotes a 3 to 6 membered heterocycloaliphatic residue, the 3 to 10 membered heterocycloaliphatic residue is linked via a carbon atom,
   R⁶¹⁰ denotes H or an unsubstituted C₁₋₄-aliphatic residue, preferably represents an unsubstituted C₁₋₄-aliphatic residue, or denotes H, methyl, ethyl, n-propyl, 2-propyl, n-butyl, isobutyl, sec-butyl or tert-butyl, preferably denotes methyl, ethyl, n-propyl, 2-propyl, n-butyl, isobutyl, sec-butyl or tert-butyl, or
   R⁶⁰⁹ and R⁶¹⁰ form together with the nitrogen atom connecting them a morpholinyl, piperidinyl, pyrrolidinyl, or azetidinyl, in each case unsubstituted or mono- or polysubstituted with at least one substituent selected from the group consisting of F, Cl, Br, I, OH, an O—C₁₋₄ aliphatic residue, and a C₁₋₄-aliphatic residue
      wherein the C₁₋₄-aliphatic residue in each case may be unsubstituted or mono- or polysubstituted with OH or an unsubstituted O—C₁₋₄-aliphatic residue.

Most preferred,
R⁶⁰⁶ denotes ethyl, n-propyl, 2-propyl, n-butyl, isobutyl, sec.-butyl, tert.-butyl, n-pentyl, isopentyl, neopentyl, n-hexyl, ethenyl or propenyl (—CH₂CH═CH₂, —CH═CH—CH₃, —C(═CH₂)—CH₃), in each case unsubstituted or mono- or polysubstituted with at least one substituent selected from the group consisting of F, Cl, OH, an O—C₁₋₄-aliphatic residue, CF₃, and a C₁₋₄-aliphatic residue, preferably in each case unsubstituted or mono- or polysubstituted with at least one substituent selected from the group consisting of F, Cl, and an O—C₁₋₄-aliphatic residue, preferably O-methyl, more preferably in each case unsubstituted,
   wherein the C₁₋₄-aliphatic residue in each case is unsubstituted,
or denotes cyclopropyl, cyclobutyl, cyclopentyl and cyclohexyl, piperidinyl, tetrahydrofuranyl, or tetrahydropyranyl, preferably denotes cyclopropyl or tetrahydropyranyl, more preferably cyclopropyl, in each case unsubstituted or mono- or polysubstituted with at least one substituent selected from the group consisting of F, Cl, OH, an O—C₁₋₄-aliphatic residue, CF₃, and a C₁₋₄-aliphatic residue, preferably in each case unsubstituted or mono- or polysubstituted with at least one substituent selected from the group consisting of F, Cl, and an O—C₁₋₄-aliphatic residue, preferably O-methyl, more preferably in each case unsubstituted,
   wherein the C₁₋₄-aliphatic residue in each case is unsubstituted,
and wherein cyclopropyl, cyclobutyl, cyclopentyl and cyclohexyl, piperidinyl, tetrahydrofuranyl, and tetrahydropyranyl may in each case be optionally bridged via an unsubstituted C₁₋₄ aliphatic group, preferably via an unsubstituted C₁₋₂ aliphatic group, on the condition that if R⁶⁰⁶ denotes piperidinyl, tetrahydrofuranyl, or tetrahydropyranyl, piperidinyl, tetrahydrofuranyl, or tetrahydropyranyl is linked via a carbon atom, or
R⁶⁰⁶ denotes S—R⁶⁰⁷ or O—R⁶⁰⁸,
   wherein
   R⁶⁰⁷ and R⁶⁰⁸ in each case denote methyl, ethyl, n-propyl, 2-propyl, n-butyl, isobutyl, sec.-butyl, tert.-butyl, n-pentyl, isopentyl, neopentyl, n-hexyl, ethenyl and propenyl (—CH₂CH═CH₂, —CH═CH—CH₃, —C(═CH₂)—CH₃), in each case unsubstituted or mono- or polysubstituted with at least one substituent selected from the group consisting of F, Cl, OH, N(C₁₋₄ aliphatic residue)₂ and an O—C₁₋₄-aliphatic residue,
      wherein the C₁₋₄-aliphatic residue in each case is unsubstituted,
   or in each case denote cyclopropyl, cyclobutyl, cyclopentyl cyclohexyl, oxetanyl, piperidinyl, tetrahydrofuranyl, or tetrahydropyranyl, preferably cyclopropyl or oxetanyl, in each case unsubstituted or mono- or polysubstituted with at least one substituent selected from the group consisting of F, Cl, OH, an O—C₁₋₄-aliphatic residue, CF₃, and a C₁₋₄-aliphatic residue, preferably in each case unsubstituted or mono- or polysubstituted with at least one substituent selected from the group consisting of F, Cl, and an O—C₁₋₄-aliphatic residue, more preferably in each case unsubstituted,
      wherein the C₁₋₄-aliphatic residue in each case is unsubstituted,
   and wherein cyclopropyl, cyclobutyl, cyclopentyl and cyclohexyl, oxetanyl, piperidinyl, tetrahydrofuranyl, and tetrahydropyranyl may in each case be optionally bridged via an unsubstituted C₁₋₄ aliphatic group,
   on the condition that if R⁶⁰⁷ or R⁶⁰⁸ denotes piperidinyl, oxetanyl, tetrahydrofuranyl, or tetrahydropyranyl, each of these residues is linked via a carbon atom, or
R⁶⁰⁶ denotes N(R⁶⁰⁹R⁶¹⁰),
   wherein
   R⁶⁰⁹ denotes a C₁₋₆-aliphatic residue, unsubstituted or mono- or polysubstituted with at least one substituent selected from the group consisting of F, Cl, ═O, OH, and O-methyl, preferably unsubstituted or mono- or polysubstituted with at least one substituent selected from the group consisting of F, Cl, and O-methyl, more preferably unsubstituted or mono- or polysubstituted with at least one substituent selected from the group consisting of F and O-methyl, preferably denotes an unsubstituted C₁₋₆-aliphatic residue, more preferably selected from the group consisting of methyl, ethyl, n-propyl, 2-propyl, n-butyl, isobutyl, sec.-butyl, tert.-butyl, n-pentyl, isopentyl, neopentyl, and n-hexyl,
   R⁶¹⁰ denotes H, methyl, ethyl, n-propyl, 2-propyl, n-butyl, isobutyl, sec-butyl or tert-butyl, preferably methyl, ethyl, n-propyl, 2-propyl, n-butyl, isobutyl, sec-butyl or tert-butyl, more preferably methyl or ethyl, or
   R⁶⁰⁹ and R⁶¹⁰ form together with the nitrogen atom connecting them a morpholinyl, piperidinyl, pyrrolidinyl, or azetidinyl, in each case unsubstituted or mono- or polysubstituted with at least one substituent selected from the group consisting of F, Cl, Br, I, OH, an O—C₁₋₄ aliphatic residue, and a C₁₋₄-aliphatic residue, more preferably unsubstituted or mono- or polysubstituted with at least one substituent selected from the group consisting of F, Cl and a O—C₁₋₄ aliphatic residue, preferably form together with the nitrogen atom connecting them a morpholinyl, piperidinyl, pyrrolidinyl, or azetidinyl, in each case unsubstituted.

In particular,
R⁶⁰⁶ denotes ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), ethenyl or propenyl (—CH₂CH═CH₂, —CH═CH—CH₃, —C(═CH₂)—CH₃), CH₂—OCH₃, C₂H₄—OCH₃, C₃H₆—OCH₃, cyclopropyl, cyclobutyl, or tetrahydropyranyl, in each case unsubstituted, or
R⁶⁰⁶ denotes S—R⁶⁰⁷ or O—R⁶⁰⁸,
   wherein R⁶⁰⁷ and R⁶⁰⁸ in each case denote methyl, ethyl, n-propyl, 2-propyl, n-butyl, isobutyl, sec.-butyl, tert.-butyl, n-pentyl, isopentyl, neopentyl, or n-hexyl, in each case unsubstituted or mono- or polysubstituted with at least one substituent selected from the group consisting of F, Cl, OH, a N(C₁₋₄ aliphatic residue)₂, and an O—C₁₋₄-aliphatic residue, preferably with at least one substituent selected from the group consisting of F, OH, N(CH₃)₂, O-methyl and O-ethyl, or in each case denote CH₂-cyclopropyl or oxetanyl, preferably, R⁷ and R⁸ in each case denote methyl, ethyl, n-propyl, 2-propyl, n-butyl, isobutyl, sec.-butyl, tert.-butyl, n-pentyl, isopentyl, neopentyl, or n-hexyl, CH₂—CH₂—F, CH₂CHF₂, CH2-OCH3, CH₂CH₂—OCH₃, CH₂CH₂—N(CH₃)₂, CH₂-cyclopropyl or oxetanyl,
      wherein the C₁₋₄-aliphatic residue in each case is unsubstituted, or
R⁶⁰⁶ denotes N(R⁶⁰⁹R⁶¹⁰),
   wherein
   R⁶⁰⁹ denotes methyl, ethyl, C(═O)—CH₃, n-propyl, 2-propyl, n-butyl, isobutyl, sec.-butyl, tert.-butyl, n-pentyl, isopentyl, neopentyl, or n-hexyl,
   R⁶¹⁰ denotes H, methyl or ethyl, preferably methyl or ethyl, or
   R⁶⁰⁹ and R⁶¹⁰ form together with the nitrogen atom connecting them a morpholinyl, piperidinyl, pyrrolidinyl, or azetidinyl, in each case unsubstituted.

Particularly preferred is a compound according to general formula (6-I), wherein
R⁶⁰¹ represents the partial structure (6-T1),

—(CR^{612a}R^{612b})ₘ₆—R^{612c} (6-T1),
wherein
m6 is 0, 1 or 2, preferably 0 or 2, more preferably 2, and
R^{612a} and R^{612b} each independently of one another represent H, F, OH, CH₃ or OCH₃ or together denote ═O, more preferably H, F, OH or CH₃, even more preferably H,
R^{612c} denotes a C₁₋₄ aliphatic residue, preferably methyl, ethyl, n-propyl, 2-propyl, n-butyl, isobutyl, sec.-butyl, or tert.-butyl, unsubstituted or mono- or polysubstituted with at least one substituent selected from the group consisting of F, Cl, Br, I, CN, OH, S(═O)₂—CH₃, an unsubstituted O—C₁₋₄ aliphatic residue, preferably O-methyl and O-tert.-butyl, and CF₃, preferably denotes a C₁₋₄ aliphatic residue, preferably methyl, ethyl, n-propyl, 2-propyl, n-butyl, isobutyl, sec.-butyl, or tert.-butyl, unsubstituted or mono- or polysubstituted with at least one substituent selected from the group consisting of F, Cl, Br, I, an unsubstituted O—C₁₋₄ aliphatic residue, preferably O-methyl and O-tert.-butyl, and CF₃,
or denotes a C₃₋₁₀-cycloaliphatic residue or a 3 to 10 membered heterocycloaliphatic residue, preferably cyclopropyl, cyclopentyl, cyclohexyl, morpholinyl, oxetanyl or tetrahydropyranyl, in each case unsubstituted or mono- or polysubstituted with at least one substituent selected from the group consisting of F, Cl, Br, I, an unsubstituted O—C₁₋₄ aliphatic residue, preferably O-methyl and O-ethyl, CF₃, and an unsubstituted C₁₋₄-aliphatic residue, preferably methyl or ethyl, or
wherein
m6 is 0 or 2, more preferably 0, and
R^{612a} and R^{612b} each independently of one another represent H, F, CH₃ or OCH₃; and
R^{612c} denotes an aryl or heteroaryl, preferably phenyl or pyridyl, in each case unsubstituted or mono- or polysubstituted with at least one substituent selected from the group consisting of F, Cl, Br, I, OH, an O—C₁₋₄ aliphatic residue, OCF₃, OCF₂H, CH₂—OH, CH₂—OCH₃, S(═O)₂—CH₃, SCF₃, NO₂, N(CH₃)₂,

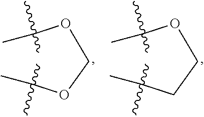 [SMILES: *OCO* ; *CCO*]

CF₃, CN, a C₁₋₄-aliphatic residue, C(═O)—CH₃, C(═O)—C₂H₅, C(═O)—O—CH₃, C(═O)—O—C₂H₅ and phenyl, preferably denotes an aryl or heteroaryl, preferably phenyl or pyridyl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
R⁶⁰² represents F; Cl; Br; I; CN; CF₃; NO₂; OCF₃; SCF₃; methyl; ethyl; n-propyl; iso-propyl; n-butyl; sec.-butyl; tert.-butyl; CH₂—OH; CH₂—O—CH₃; CH₂—CH₂—OH; CH₂—CH₂—OCH₃; O-methyl; O-ethyl; O—(CH₂)₂—O—CH₃; O—(CH₂)₂—OH; S-Methyl; S-Ethyl; cyclopropyl, cyclobutyl, cyclopentyl, or cyclohexyl; preferably represents F; Cl; Br; I; CN; CF₃; NO₂; OCF₃; SCF₃; methyl; ethyl; n-propyl; iso-propyl; n-butyl; sec.-butyl; tert.-butyl; CH₂—OH; O-methyl; O-ethyl; O—(CH₂)₂—O—CH₃; O—(CH₂)₂—OH; S-Methyl; S-Ethyl; cyclopropyl, cyclobutyl, cyclopentyl, or cyclohexyl,
R⁶⁰³ represents H; F; Cl; Br; I; CN; CF₃; SCF₃; NO₂; OCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; or S-Ethyl,
R⁶⁰⁴ represents the partial structure (6-T2),

—(CR^{613a}R^{613b})ₙ₆—R^{613c} (6-T2),
wherein
n6 denotes 0, 1, 2 or 3, preferably denotes 1 or 2, more preferably denotes 1,
R^{613a} and R^{613b} each independently of one another represent H, F, CH₃ or OCH₃, or together denote ═O, preferably each independently of one another represent H or CH₃, more preferably H,
R^{613c} denotes a C₁₋₄ aliphatic residue, preferably methyl, ethyl, n-propyl, 2-propyl, n-butyl, isobutyl, sec.-butyl, or tert.-butyl, unsubstituted or mono- or polysubstituted with at least one substituent selected from the group consisting of F, Cl, Br, I, ═O, an unsubstituted O—C₁₋₄ aliphatic residue, and CF₃,
or denotes cyclopropyl, cyclobutyl, cyclopentyl or cyclohexyl, pyrrolidinyl, morpholinyl, piperazinyl, piperidinyl and tetrahydropyranyl, more preferably tetrahydropyranyl or morpholinyl, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes an aryl or heteroaryl, preferably phenyl or pyridyl, more preferably phenyl, in each case unsubstituted or mono- or polysubstituted with at least one substituent selected from the group consisting of F, Cl, Br, I, OH, an O—C₁₋₄ aliphatic residue, OCF₃, CF₃, CN, and a C₁₋₄-aliphatic residue,
R⁶⁰⁵ denotes H, methyl or ethyl, C₂H₄OCH₃ or C₃H₆OCH₃, more preferably H or methyl, even more preferably methyl, or
R⁶⁰⁴ and R⁶⁰⁵ form together with the nitrogen atom connecting them a morpholinyl, piperidinyl, pyrrolidinyl, azetidinyl, oxazepanyl, tetrahydroquinolinyl, tetrahydroisoquinolinyl, dihydroindolinyl, or dihydroisoindolyl, preferably a morpholinyl, piperidinyl, pyrrolidinyl, azetidinyl, oxazepanyl, tetrahydroquinolinyl, tetrahydroisoquinolinyl, thiomorpholinyl, azepanyl,

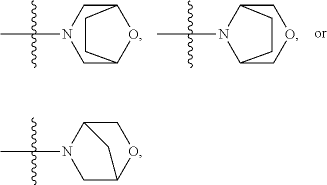 [SMILES: *N1CC2CCC(C1)O2 ; *N1C2CCC1COC2 ; "or" ; *N1CC2CC1CO2]

tetrahydroimidazo[1,2-a]pyrazinyl, octahydropyrrolo[1,2-a]pyrazinyl,

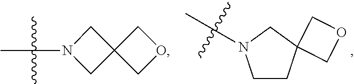 [SMILES: *N1CC2(COC2)C1 ; *N1CCC2(COC2)C1]

dihydroindolinyl, or dihydroisoindolyl, preferably a a morpholinyl, piperidinyl, pyrrolidinyl, azetidinyl, oxazepanyl, tetrahydroquinolinyl, tetrahydroisoquinolinyl, dihydroindolinyl, or dihydroisoindolyl, more preferably a morpholinyl, oxazepanyl, tetrahydroquinolinyl, or tetrahydroisoquinolinyl, in each case unsubstituted or mono- or polysubstituted with at least one substituent selected from the group consisting of F, Cl, OH, ═O, C(═O)—OH, O-methyl, O-ethyl, OCF₃, SCF₃, CF₃, C(═O)—CH₃, C(═O)—OCH₃, CH₂CF₃, CH₂OH, CH₂—OCH₃, CH₂CH₂—OCH₃, methyl, ethyl, n-propyl, 2-propyl, cyclopropyl, and cyclobutyl, preferably selected from the group consisting of F, Cl, OH, ═O, C(═O)—OH, O-methyl, O-ethyl, OCF₃, SCF₃, CF₃, methyl, ethyl, n-propyl, 2-propyl, cyclopropyl, and cyclobutyl,
R⁶⁰⁶ denotes ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), ethenyl or propenyl (—CH₂CH═CH₂, —CH═CH—CH₃, —C(═CH₂)—CH₃), CH₂—OCH₃, C₂H₄—OCH₃, C₃H₆—OCH₃, cyclopropyl, cyclobutyl, or tetrahydropyranyl, in each case unsubstituted, or
R⁶⁰⁶ denotes S—R⁶⁰⁷ or O—R⁶⁰⁸,
   wherein R⁶⁰⁷ and R⁶⁰⁸ in each case denote methyl, ethyl, n-propyl, 2-propyl, n-butyl, isobutyl, sec.-butyl, tert.-butyl, n-pentyl, isopentyl, neopentyl, or n-hexyl, in each case unsubstituted or mono- or polysubstituted with at least one substituent selected from the group consisting of F, Cl, OH, a N(C₁₋₄ aliphatic residue)₂, and an O—C₁₋₄-aliphatic residue, preferably with at least one substituent selected from the group consisting of F, OH, N(CH₃)₂, O-methyl and O-ethyl, or in each case denote CH₂-cyclopropyl or oxetanyl, preferably, R⁷ and R⁸ in each case denote methyl, ethyl, n-propyl, 2-propyl, n-butyl, isobutyl, sec.-butyl, tert.-butyl, n-pentyl, isopentyl, neopentyl, or n-hexyl, CH₂—CH₂—F, CH₂CHF₂, CH2-OCH3, CH₂CH₂—OCH₃, CH₂CH₂—N(CH₃)₂, CH₂-cyclopropyl or oxetanyl,
      wherein the C₁₋₄-aliphatic residue in each case is unsubstituted, or
R⁶⁰⁶ denotes N(R⁶⁰⁹R⁶¹⁰),
   wherein
   R⁶⁰⁹ denotes methyl, C(═O)—CH₃, ethyl, n-propyl, 2-propyl, n-butyl, isobutyl, sec.-butyl, tert.-butyl, n-pentyl, isopentyl, neopentyl, or n-hexyl,
   R⁶¹⁰ denotes H, methyl or ethyl, preferably methyl or ethyl, or
   R⁶⁰⁹ and R⁶¹⁰ form together with the nitrogen atom connecting them a morpholinyl, piperidinyl, pyrrolidinyl, or azetidinyl, in each case unsubstituted.

In another particularly preferred embodiment of the compound according to general formula (6-I),
R⁶⁰¹ represents phenyl or pyridyl, preferably phenyl, in each case unsubstituted or mono- or disubstituted with at least one substituent selected from the group consisting of F, Cl, Br, OH, OCH₃, OCF₃, CF₃, and CH₃,
R⁶⁰² represents H; CF₃; methyl; ethyl; iso-propyl; O-methyl; or cyclopropyl,
R⁶⁰³ represents H; F; Cl; Br; I; CN; CF₃; methyl; or O-methyl,
R⁶⁰⁴ and R⁶⁰⁵ form together with the nitrogen atom connecting them a morpholinyl, piperidinyl, pyrrolidinyl, azetidinyl, oxazepanyl, tetrahydroquinolinyl, tetrahydroisoquinolinyl, dihydroindolinyl, or dihydroisoindolyl, in each case unsubstituted,
R⁶⁰⁶ denotes ethyl, n-propyl, 2-propyl (iso-propyl), tert.-butyl, cyclopropyl, cyclobutyl or cyclopentyl or tetrahydropyranyl, or
R⁶⁰⁶ denotes S—R⁶⁰⁷ or O—R⁶⁰⁸,
   wherein R⁶⁰⁷ and R⁶⁰⁸ in each case denote methyl, ethyl, 2-propyl, or tert-butyl, or
R⁶⁰⁶ denotes N(R⁶⁰⁹R⁶¹⁰),
   wherein
   R⁶⁰⁹ denotes methyl, ethyl, n-propyl, 2-propyl, or tert-butyl,
   R⁶¹⁰ denotes H, methyl or ethyl, preferably methyl or ethyl, or
   R⁶⁰⁹ and R⁶¹⁰ form together with the nitrogen atom connecting them a morpholinyl, piperidinyl, pyrrolidinyl, or azetidinyl.

Another embodiment of the present invention relates to a compound of general formula (I), wherein the compound is selected from a compound according to general formula (7-I).

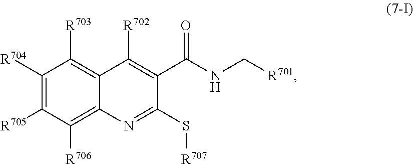

wherein
R⁷⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
   R⁷⁰² represents H; F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
   R⁷⁰³, R⁷⁰⁴, R⁷⁰⁵ and R⁷⁰⁶ each independently of one another represent H; F; Cl; Br; I; CN; CF₃; C(═O)H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂—C₁₋₄-aliphatic residue, a N(C₁₋₄ aliphatic residue)-C(═O)—C₁₋₄ aliphatic residue, or a N(C₁₋₄ aliphatic residue)-S(═O)₂—C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁷⁰⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁷⁰⁷ denotes a 3 to 10 membered heterocycloaliphatic residue, the 3 to 10 membered heterocycloaliphatic residue is linked via a carbon atom,

in which an “aliphatic group” and “aliphatic residue” can in each case be branched or unbranched, saturated or unsaturated,

in which a “cycloaliphatic residue” and a “heterocycloaliphatic residue” can in each case be saturated or unsaturated,

in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C_{1- 4}-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;

in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

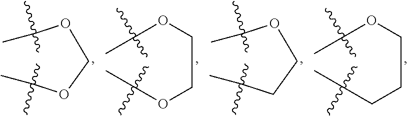 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

The compounds according to general formula (7-I) are known from WO 2010/102811 A1, which is fully included by reference.

Preferred embodiments of the compound according to general formula (7-I) have general formulae (7-Ia), (7-Ib), (7-Ic) or (7-Id):

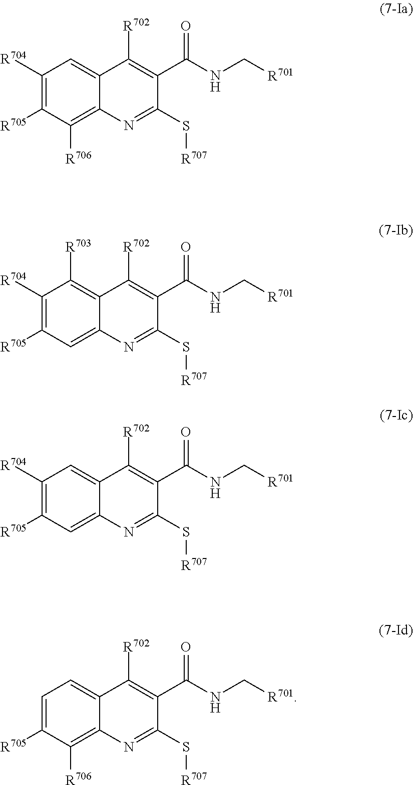

Another preferred embodiment of present invention is a compound according to general formula (7-I), wherein
R⁷⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

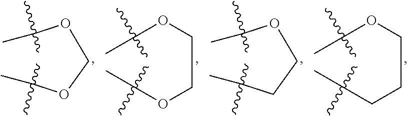 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH,
R⁷⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, preferably represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and an O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
R⁷⁰³, R⁷⁰⁴, R⁷⁰⁵ and R⁷⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, CI, Br, I, ═O, OH, a C₁₋₄-aliphatic residue and a O—C₁₋₄-aliphatic residue, and in each case optionally bridged via an unsubstituted C₁₋₄ aliphatic group,

preferably on the condition that at least one of R⁷⁰³, R⁷⁰⁴, R⁷⁰⁵ and R⁷⁰⁶ is ≠H,
R⁷⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
   and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
on the condition that if R⁷⁰⁷ denotes a 3 to 10 membered heterocycloaliphatic residue, the 3 to 10 membered heterocycloaliphatic residue is linked via a carbon atom.

In a preferred embodiment of the compound according to general formula (2-I), the residue
R⁷⁰¹ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

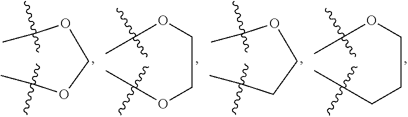 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH.

In a further preferred embodiment of the compound according to general formula (7-I), the residue
R⁷⁰¹ represents the partial structure (7-T1)

—(CR^{708a}R^{708b})ₘ₇—R^{708C} (7-T1),

wherein
m7 denotes 0, 1, 2, 3 or 4, preferably denotes 0, 1, or 2,
R^{708a} and R^{708b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O,
   preferably each independently of one another represent H, F, Cl, Br, I, NH₂, a NH(C₁₋₄ aliphatic residue), a N(C₁₋₄ aliphatic residue)₂, OH, O—C₁₋₄ aliphatic residue or a C₁₋₄ aliphatic residue,
   more preferably each independently of one another represent H, F, Cl, Br, I, an O—C₁₋₄ aliphatic residue or a C₁₋₄ aliphatic residue, even more preferably each independently of one another represent H, F, an O—C₁₋₄ aliphatic residue or a C₁₋₄ aliphatic residue, and
R^{708c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes a C₃₋₁₀-cycloaliphatic residue or a 3 to 10 membered heterocycloaliphatic residue, preferably when m2 is #0, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
or denotes—preferably when m2 is =0—an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

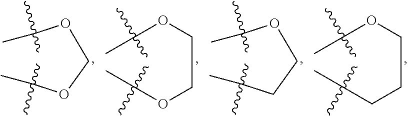 [SMILES: *OCO* ; *OCCO* ; *CCO* ; *CCCO*]

benzyl, phenyl, thienyl, pyridyl, furyl, thiazolyl and oxazolyl,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

Preferably,
R⁷⁰¹ represents the partial structure (7-T1),
   wherein
   m7 denotes 0, 1, or 2,
   R^{708a} and R^{708b} each independently of one another represent H, F, Cl, Br, I, an O—C₁₋₄ aliphatic residue or a C₁₋₄ aliphatic residue,
   preferably each independently of one another represent H, F, a O—C₁₋₂ aliphatic residue or a C₁₋₂ aliphatic residue, and
   R^{708c} denotes a C₁₋₄ aliphatic residue, unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue, C(═O)—OH, a C₃₋₆ cycloaliphatic residue, and a 3 to 6 membered heterocycloaliphatic residue,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH,
   or denotes—preferably when m7 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl or oxazolyl,
      wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
      wherein benzyl, phenyl, thienyl, pyridyl, furyl, thiazolyl and oxazolyl may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
      wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH.

More preferably,
R⁷⁰¹ represents the partial structure (7-T1),
   wherein
m7 denotes 0, 1, or 2,
R^{708a} and R^{708b} each independently of one another represent H, F, Cl, Br, I, an O—C₁₋₄ aliphatic residue or a C₁₋₄ aliphatic residue,
preferably each independently of one another represent H, F, a O—C₁₋₂ aliphatic residue or a C₁₋₂ aliphatic residue, and
R^{708c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue,
or denotes—preferably when m7 is =0—an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl,
   wherein benzyl, phenyl, thienyl and pyridyl, may in each case may be unsubstituted or mono- or polysubstituted, preferably unsubstituted or mono- or disubstituted with at least one substituent selected from the group consisting of F, CI, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃, a C₁₋₄-aliphatic residue and C(═O)—OH.

In a further preferred embodiment of the compound according to general formula (7-I), the residue
R⁷⁰¹ represents the partial structure (7-T1),
   wherein
m7 is 0, 1 or 2 and
R^{708a} and R^{708b} each independently of one another represent H, F, a O—C₁₋₄ aliphatic residue or a C₁₋₄ aliphatic residue; preferably H, F, CH₃ or OCH₃;
R^{708c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
   wherein
m7 is 0,
R^{708a} and R^{708b} each independently of one another represent H, F, a O—C₁₋₄ aliphatic residue or a C₁₋₄ aliphatic residue; preferably H, F, CH₃ or OCH₃; and
R^{708c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃.

Particularly preferred is a compound according to general formula (7-I) which has the following general formula (7-Ie):

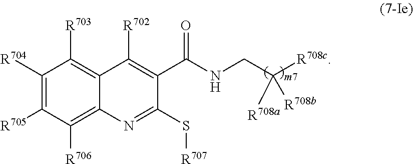

In a preferred embodiment of the compound according to general formula (7-I), the residue
R⁷⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted.

Preferably,
R⁷⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

More preferably,
R⁷⁰² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue,
   wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue,
cyclopropyl, cyclobutyl, cyclopentyl, cyclohexyl, pyrrolidinyl, piperazinyl, 4-methylpiperazinyl, morpholinyl, or piperidinyl, preferably cyclopropyl, cyclobutyl, cyclopentyl or cyclohexyl, in each case unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue,
   and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted O—C₁₋₄-aliphatic residue.

Even more preferably,
R⁷⁰² represents H; F; Cl; Br; I; CN; CF₃; NO₂; OCF₃; SCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; S-Ethyl; cyclopropyl, cyclobutyl, cyclopentyl, and cyclohexyl.

Still more preferably,
R⁷⁰² is selected from the group consisting of H; F; Cl; CF₃; CN; SCF₃; OCF₃; CH₃; C₂H₅; n-propyl; iso-propyl; t-butyl; cyclopropyl; O—CH₃ and O—C₂H₅;

In particular,
R⁷⁰² is selected from the group consisting of H; F; Cl; CF₃; CH₃; C₂H₅, iso-propyl; cyclopropyl; and O—CH₃;

In a particular preferred embodiment of the compound according to general formula (7-I), the residue
R⁷⁰² is ≠H.

In a preferred embodiment of the compound according to general formula (7-I), the residues
R⁷⁰³, R⁷⁰⁴, R⁷⁰⁵ and R⁷⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂—C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group,

preferably on the condition that at least one of R⁷⁰³, R⁷⁰⁴, R⁷⁰⁵ and R⁷⁰⁶ is ≠H.

Preferably,
R⁷⁰³, R⁷⁰⁴, R⁷⁰⁵ and R⁷⁰⁶ each independently of one another represent H; F; Cl; Br; I; CN; CF₃; OCF₃; SCF₃; C(═O)H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a O—C(═O)—C₁₋₄-aliphatic residue, a S—C₁₋₄-aliphatic residue, a S(═O)₂—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group,

preferably on the condition that at least one of R⁷⁰³, R⁷⁰⁴, R⁷⁰⁵ and R⁷⁰⁶ is ≠H.

More preferably,
R⁷⁰³, R⁷⁰⁴, R⁷⁰⁵ and R⁷⁰⁶ each independently of one another represent H; F; Cl; Br; I; CN; CF₃; OCF₃; SCF₃; C(═O)H; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a S—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue, unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group,

preferably on the condition that at least one of R⁷⁰³, R⁷⁰⁴, R⁷⁰⁵ and R⁷⁰⁶ is ≠H.

In a further preferred embodiment of the present invention,
R⁷⁰³, R⁷⁰⁴, R⁷⁰⁵ and R⁷⁰⁶ each independently of one another are selected from the group consisting of H; F; Cl; Br; I; NO₂; CF₃; CN; OCF₃; SCF₃; a (C═O)—C₁₋₄ aliphatic residue, a C₁₋₄ aliphatic residue, O—C₁₋₄ aliphatic residue, a S—C₁₋₄ aliphatic residue, wherein the C₁₋₄-aliphatic residue in each case may be unsubstituted or mono- or polysubstituted with at least one substituent selected from the group consisting of F, Cl, and O—CH₃;

preferably on the condition that at least one of R⁷⁰³, R⁷⁰⁴, R⁷⁰⁵ and R⁷⁰⁶ is ≠H.

Preferably,
R⁷⁰³, R⁷⁰⁴, R⁷⁰⁵ and R⁷⁰⁶ each independently of one another are selected from the group consisting of H; F; Cl; Br; I; NO₂; CF₃; CN; OCF₃; SCF₃; methyl; ethyl; n-propyl; iso-propyl; n-butyl; sec.-butyl; tert.-butyl; cyclopropyl; C(═O)-methyl; C(═O)-ethyl; (C═O)-isopropyl; (C═O)-t-butyl; O-methyl; O-ethyl; O-isopropyl; O-t-butyl; O—(CH₂)₂—O—CH₃; S-Methyl; S-Ethyl;

preferably on the condition that at least one of R⁷⁰³, R⁷⁰⁴, R⁷⁰⁵ and R⁷⁰⁶ is ≠H.

In particular,
R⁷⁰³, R⁷⁰⁴, R⁷⁰⁵ and R⁷⁰⁶ are each independently of one another are selected from the group consisting of H; F; Cl; Br; I; NO₂; CF₃; CN; (C═O)-methyl; (C═O)-ethyl; (C═O)-isopropyl; (C═O)-t-butyl; methyl; ethyl; isopropyl; t-butyl; O-methyl; O-Ethyl; O-isopropyl; O-t-butyl; OCF₃; S-methyl; S-ethyl; and SCF₃;

preferably on the condition that at least one of R⁷⁰³, R⁷⁰⁴, R⁷⁰⁵ and R⁷⁰⁶ is ≠H.

More particularly,
R⁷⁰³, R⁷⁰⁴, R⁷⁰⁵ and R⁷⁰⁶ are each independently of one another selected from the group consisting of H; F; Cl; Br; CF₃; CN; OCF₃ and NO₂; preferably on the condition that at least one of R⁷⁰³, R⁷⁰⁴, R⁷⁰⁵ and R⁷⁰⁶ is ≠H.

Most preferred,
R⁷⁰³, R⁷⁰⁴ and R⁷⁰⁶ each independently of one another are selected from the group consisting of H and F; and
R⁷⁰⁵ denotes F; Br; CF₃; OCF₃; CN; or NO₂;

In a particular preferred embodiment of the compound according to general formula (7-I) at least one of the residues R⁷⁰³, R⁷⁰⁴, R⁷⁰⁵ and R⁷⁰⁶ is ≠H.

In a preferred embodiment of the compound according to general formula (7-I), the residue
R⁷⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH,
on the condition that if R²⁰⁷ denotes a 3 to 10 membered heterocycloaliphatic residue, the 3 to 10 membered heterocycloaliphatic residue is linked via a carbon atom.

In a further preferred embodiment of the compound according to general formula (7-I), the residue
R⁷⁰⁷ denotes a C₁₋₁₀-aliphatic residue, preferably a C₁₋₈-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
   wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue,
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue.
on the condition that if R⁷⁰⁷ denotes a 3 to 10 membered heterocycloaliphatic residue, the 3 to 10 membered heterocycloaliphatic residue is linked via a carbon atom.

Preferably,
R⁷⁰⁷ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
and wherein the C₃₋₁₀-cycloaliphatic residue or the 3 to 10 membered heterocycloaliphatic residue may in each case optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue.
on the condition that if R⁷⁰⁷ denotes a 3 to 10 membered heterocycloaliphatic residue, the 3 to 10 membered heterocycloaliphatic residue is linked via a carbon atom.

More preferably,
R⁷⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, CF₃, a C(═O)—O—C₁₋₄-aliphatic residue, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue is bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue.

Even more preferably,
R⁷⁰⁷ denotes a C₁₋₈-aliphatic residue, preferably a C₁₋₆-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue
   wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and
wherein the C₃₋₁₀-cycloaliphatic residue or the 3 to 10 membered heterocycloaliphatic residue is in each case bridged via a unsubstituted C₁₋₈ aliphatic group, preferably an unsubstituted C₁₋₄ aliphatic group.

Still more preferably,
R⁷⁰⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue
   wherein the C₁₋₄-aliphatic residue in each case is unsubstituted,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue,
   wherein the C₁₋₄-aliphatic residue in each case may be unsubstituted or mono- or polysubstituted with OH or an unsubstituted O—C₁₋₄-aliphatic residue.
and wherein the C₃₋₁₀-cycloaliphatic residue or the 3 to 10 membered heterocycloaliphatic residue is in each case bridged via a unsubstituted C₁₋₄ aliphatic group

In particular,
R⁷⁰⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue
   wherein the C₁₋₄-aliphatic residue in each case is unsubstituted.

Most preferred,
R⁷⁰⁷ denotes a C₁₋₆-aliphatic residue, unsubstituted or mono- or polysubstituted with at least one substituent selected from the group consisting of F, CF₃, Cl, OH, and O-methyl.

Preferred is also a compound according to general formula (7-I), wherein
R⁷⁰¹ represents the partial structure (7-T1),

—(CR^{708a}R^{708b})ₘ₇—R^{708c} (7-T1),
wherein
m7 is 0, 1 or 2 and
R^{708a} and R^{708b} each independently of one another represent H, F, a O—C₁₋₄ aliphatic residue or a C₁₋₄ aliphatic residue; preferably H, F, CH₃ or OCH₃;
R^{708c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
wherein
m7 is 0,
R^{708a} and R^{708b} each independently of one another represent H, F, a O—C₁₋₄ aliphatic residue or a C₁₋₄ aliphatic residue; preferably H, F, CH₃ or OCH₃; and
R^{708c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
   wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃,
R⁷⁰² is selected from the group consisting of H; F; Cl; CF₃; CH₃; C₂H₅, iso-propyl; cyclopropyl; and O—CH₃;
R⁷⁰³, R⁷⁰⁴, R⁷⁰⁵ and R⁷⁰⁶ are each independently of one another selected from the group consisting of H; F; Cl; Br; CF₃; CN; OCF₃ and NO₂;

preferably on the condition that at least one of R²⁰³, R²⁰⁴, R²⁰⁵ and R²⁰⁶ is ≠H,
R⁷⁰⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue
   wherein the C₁₋₄-aliphatic residue in each case is unsubstituted.

Particularly preferred is also a compound according to general formula (7-I), wherein
R⁷⁰¹ represents aryl, preferably phenyl, or heteroaryl, preferably pyridyl or thienyl, in each case unsubstituted or mono- or disubstituted with at least one substituent selected from the group consisting of F, Cl, Br, I, OH, OCH₃, OCF₃, CF₃, CN, and CH₃,
preferably represents phenyl, unsubstituted or mono- or disubstituted with at least one substituent selected from the group consisting of F, Cl, Br, I, OH, OCH₃, OCF₃, CF₃, CN, and CH₃;
R⁷⁰² is selected from the group consisting of H, F, Cl, CF₃, CH₃, C₂H₅, iso-propyl, cyclopropyl, and O—CH₃; preferably is selected from the group consisting of CH₃, C₂H₅, OCH₃ and CF₃;
R⁷⁰³, R⁷⁰⁴, R⁷⁰⁵ and R⁷⁰⁶ are each independently of one another selected from the group consisting of H, F, Cl, Br, CF₃, CN, OCF₃ and NO₂;
preferably on the condition that at least one of R⁷⁰³, R⁷⁰⁴, R⁷⁰⁵ and R⁷⁰⁶ is ≠H, more
preferably on the condition that R⁷⁰⁵ is ≠H;
R⁷⁰⁷ denotes a saturated C₁₋₄-aliphatic residue, unsubstituted or mono- or disubstituted with at least one substituent selected from the group consisting of F, Cl, Br, I, OH, O—CH₃, OCF₃, SCF₃, and CF₃.

Another aspect of the present invention further relates to at least one compound according to general formula (I) and also if appropriate of one or more pharmaceutically acceptable auxiliaries for the use in the preparation of a medicament for prophylaxis and/or treatment of disorders and/or diseases which are mediated, at least in part, by TSPO activity.

The selection of the physiologically acceptable auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, dragees, capsules, granules, pellets, drops, juices and syrups are preferably suitable for oral application; solutions, suspensions, easily reconstitutable dry preparations and also sprays are preferably suitable for parenteral, topical and inhalative application. The substituted compounds according to the invention used in the pharmaceutical composition according to the invention in a repository, in a dissolved form or in a plaster, and further agents promoting skin penetration being added if appropriate, are suitable percutaneous application preparations. Orally or percutaneously applicable preparation forms can release the respective substituted compound according to the invention also in a delayed manner.

The pharmaceutical compositions according to the invention can be prepared with the aid of conventional means, devices, methods and process known in the art, such as are described for example in “Remington's Pharmaceutical Sciences”, A. R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula (I) may vary and is for example dependent on the patient's weight or age and also on the type of application, the indication and the severity of the disorder. Conventionally, 0.001 to 100 mg/kg, preferably 0.05 to 75 mg/kg, particularly preferably 0.05 to 50 mg of at least one compound according to the invention are applied per kg of the patient's body weight.

Preference is given to at least one compound according to general formula (I) and optionally one or more pharmaceutically acceptable auxiliaries for the use in the preparation of a medicament for the prophylaxis and/or treatment of disorders and/or diseases selected from the group consisting of
(i) neurological and neurodegenerative disorders, including
   ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia;
(ii) psychiatric disorders, including
   anxiety disorders, particularly panic disorders, post traumatic stress disorder, panic and adult separation disorder, social anxiety, major depression with adult separation anxiety disorder and bipolar disorder with adult separation anxiety disorder, dysthymia, angina pectoris, suicidality and schizophrenia;
(iii) brain tumours;
(iv) immunological disorders and inflammatory diseases, including
   neuroinflammation and arthritis, encephalomyelitis and irritable bowel syndrome.

Particular preference is given to at least one compound according to general formula (I) and optionally one or more pharmaceutically acceptable auxiliaries for use in the preparation of a medicament for the prophylaxis and/or treatment of neurological and neurodegenerative disorders, including Ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia.

Particular preference is given to at least one compound according to general formula (I) and optionally one or more pharmaceutically acceptable auxiliaries for use in the preparation of a medicament for the prophylaxis and/or treatment of psychiatric disorders, including anxiety disorders, particularily panic disorders, post traumatic stress disorder, panic and adult separation disorder, social anxiety, major depression with adult separation anxiety disorder and bipolar disorder with adult separation anxiety disorder, dysthymia, angina pectoris, suicidality and schizophrenia.

Particular preference is given to at least one compound according to general formula (I) and optionally one or more pharmaceutically acceptable auxiliaries for use in the preparation of a medicament for the prophylaxis and/or treatment of brain tumours.

Particular preference is given to at least one compound according to general formula (I) and optionally one or more pharmaceutically acceptable auxiliaries for use in the preparation of a medicament for the prophylaxis and/or treatment of immunological disorders and inflammatory diseases, including neuroinflammation and arthritis, encephalomyelitis and irritable bowel syndrome.

Another aspect of the present invention is a method of treatment and/or prophylaxis of disorders and/or diseases, which are mediated, at least in part, by TSPO activity, in a mammal, preferably of disorders and/or diseases selected from the group consisting of
(i) neurological and neurodegenerative disorders, including
   ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia;
(ii) psychiatric disorders, including
   anxiety disorders, particularly panic disorders, post traumatic stress disorder, panic and adult separation disorder, social anxiety, major depression with adult separation anxiety disorder and bipolar disorder with adult separation anxiety disorder, dysthymia, angina pectoris, suicidality and schizophrenia;
(iii) brain tumours;
(iv) immunological disorders and inflammatory diseases, including
   neuroinflammation and arthritis, encephalomyelitis and irritable bowel syndrome, which comprises administering an effective amount of at least one compound of general formula (I) to the mammal.

The various, and in particular the preferred, embodiments of the first aspect of the present invention apply in analogous manner—mutatis mutandis—to the other aspects of the present invention.

### PHARMACOLOGICAL EXPERIMENTS

Evaluation of the in vitro affinity of test substances for the rat peripheral BZD receptor was performed according to Le Fur et al. (1983). Life Sci., 33: 449-457. For this purpose, rat heart membranes and [3H]PK 11195 (1-(2-chlorophenyl)-N-methyl-N-(1-methylpropyl)-3-isoquinolinecarboxamide) were used in a radioligand binding test system.

In detail, membrane homogenates of heart (20 μg protein) are incubated for 15 min at 22° C. with 0.2 nM [3H]PK 11195 in the absence or presence of the test compound in a buffer containing 50 mM Tris-HCl (pH 7.4) and 10 mM MgCl₂ (96-well plate format). Compound addition is 100× solution in solvent. Nonspecific binding is determined in the presence of 10 μM PK 11195. Following incubation, the samples are filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters are dried, then counted for radioactivity in a scintillation counter (Topcount, Packard) using a scintillation cocktail (Microscint 0, Packard). The results are expressed as a percent inhibition of the control radioligand specific binding.

The standard reference compound is PK 11195, which is tested in each experiment concurrently with the test compounds.

For analysis, the specific ligand binding to the receptors is defined as the difference between the total binding and the nonspecific binding determined in the presence of an excess of unlabelled ligand. The results are expressed as a percent of control specific binding ((measured specific binding/control specific binding)×100) obtained in the presence of the test compounds.

The data are summarized in table 1.

TABLE 1

| | | | %- |
|---|---|---|---|
| | | Source of test | Inhibition |
| # | Test Compound | compound | @ 10 μM |
| 1 | N-[(3-Fluorophenyl)-methyl]-2-isopropyl-4-methyl-7- | Example 8 in | 87 |
| | (trifluoromethyl)-quinoline-3-carboxylic acid amide | WO2012/025238 | |
| | | A1 | |
| 2 | N-(4,4-Dimethyl-pentyl)-2-ethylsulfanyl-4-methyl-6- | Example 67 in | 43 |
| | morpholin-4-yl-pyridine-3-carboxylic acid amide | WO2012/052167 | |
| | | A1 | |
| 3 | N-[(4-Chlorophenyl)-methyl]-2-ethylsulfanyl-4- | Example 124 in | 91 |
| | methyl-6-morpholin-4-yl-pyridine-3-carboxylic acid | WO2012/052167 | |
| | amide | A1 | |
| 4 | N-[(4-Chlorophenyl)-methyl]-2-methoxy-4-methyl-6- | Example 165 in | 49 |
| | morpholin-4-yl-pyridine-3-carboxylic acid amide | WO2012/052167 | |
| | | A1 | |
| 5 | 2-[[3,3-Difluoro-3-(4-fluorophenyl)-propyl]sulfanyl]- | Example 124 in | 102 |
| | N-[(4-fluorophenyl)-methyl]-pyridine-3-carboxylic | WO2010/102809 | |
| | acid amide | A1 | |
| 6 | N-(5-Bicyclo[2.2.1]heptanyl-methyl)-2-[[3,3-difluoro- | Example 115 in | 99 |
| | 3-(4-fluorophenyl)-propyl]sulfanyl]-pyridine-3- | WO2010/102809 | |
| | carboxylic acid amide | A1 | |
| 7 | N-[(4-Chlorophenyl)-methyl]-2-cyclopropyl-4- | Example 335 in | 57 |
| | methyl-6-morpholin-4-yl-pyridine-3-carboxylic acid | WO2012/052167 | |
| | mide | A1 | |
| 8 | N-[(3-Fluorophenyl)-methyl]-2-methoxy-4-methyl-6- | Example 19 in | 103 |
| | morpholin-4-yl-pyridine-3-carboxylic acid amide | WO2012/052167 | |
| | | A1 | |
| 9 | N-[(4-Fluorophenyl)-methyl]-2-methoxy-4-methyl-6- | Example 39 in | 53 |
| | morpholin-4-yl-pyridine-3-carboxylic acid amide | WO2012/052167 | |
| | | A1 | |
| 10 | N-[(3-Fluorophenyl)-methyl]-4-methyl-6-morpholin- | Example 10 in | 91 |
| | 4-yl-2-propyl-pyridine-3-carboxylic acid amide | WO2012/052167 | |
| | | A1 | |
| 11 | 2-Cyclopropyl-N-[(4-fluorophenyl)-methyl]-4-methyl- | Example 331 in | 87 |
| | 6-morpholin-4-yl-pyridine-3-carboxylic acid amide | WO2012/052167 | |
| | | A1 | |
| 12 | 2-[[3-(3,4-Difluoro-phenyl)-3,3-difluoro-propyl]- | Example 209 in | 97 |
| | sulfanyl]-N-(3-methyl-butyl)-pyridine-3-carboxylic | WO2010/102809 | |
| | acid amide | A1 | |
| 13 | 2-Ethylsulfanyl-N-[(3-fluorophenyl)-methyl]-4- | Example 2 in | 95 |
| | methyl-6-morpholin-4-yl-pyridine-3-carboxylic acid | WO2012/052167 | |
| | amide | A1 | |
| 14 | N-[(4-Chlorophenyl)-methyl]-4-methyl-2-methyl- | Example 168 in | 59 |
| | sulfanyl-6-morpholin-4-yl-pyridine-3-carboxylic acid | WO2012/052167 | |
| | amide | A1 | |
| 15 | 2-Cyclopropyl-N-[(3-fluorophenyl)-methyl]-4-methyl- | Example 330 in | 90 |
| | 6-morpholin-4-yl-pyridine-3-carboxylic acid amide | WO2012/052167 | |
| | | A1 | |
| 16 | 2-[[3,3-Difluoro-3-(4-fluorophenyl)-propyl]sulfanyl]- | Example 169 in | 96 |
| | N-(3-methyl-butyl)-pyridine-3-carboxylic acid amide | WO2010/102809 | |
| | | A1 | |
| 17 | N-[(3-Fluorophenyl)-methyl]-1,4-dimethyl-2-oxo-7- | Example 1 in | 94 |
| | (trifluoromethyl)-1H-quinoline-3-carboxylic acid | WO2012/025239 | |
| | amide | A1 | |
| 18 | N-[(3-Fluorophenyl)-methyl]-1-(2-methoxyethyl)-4- | Example 4 in | 97 |
| | methyl-2-oxo-(7-trifluoromethyl)-1H-quinoline-3- | WO2012/025239 | |
| | carboxylic acid amide | A1 | |

For the preparation of the test compounds, see referenced source.

1. A method for the treatment and/or the prophylaxis of disorders and/or diseases, wherein the disorder and/or the disease is selected from the group consisting of:
(i) neurological and neurodegenerative disorders, including ischaemic stroke, Alzheimer's disease, frontotemporal dementia, Huntington's disease, amyotrophic lateral sclerosis, Parkinson's disease, multiple sclerosis, diabetic peripheral neuropathy, chemotherapy-induced peripheral neuropathy, amyotrophic lateral sclerosis, exictotoxic brain injury, traumatic brain injury, ischaemic brain damage, cerebrovascular disorders and cerebral ischemia;
(ii) brain tumours;
(iii) immunological disorders and inflammatory diseases, including neuroinflammation and arthritis, encephalomyelitis and irritable bowel syndrome;
said method comprising administering to a patient in need thereof an effective amount therefor of a compound according to general formula (5-I), 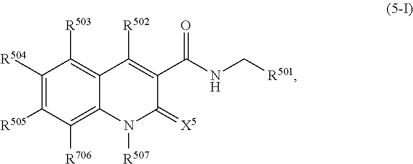
wherein
X⁵ denotes O or S,
R⁵⁰¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R⁵⁰² represents F; Cl; Br; I; CN; CF₃; C(═O)H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁵⁰³, R⁵⁰⁴, R⁵⁰⁵ and R⁵⁰⁶ each independently of one another represent H; F; Cl; Br; I; CN; CF₃; C(═O)H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂—C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂—C₁₋₄-aliphatic residue, a N(C₁₋₄ aliphatic residue)-C(═O)—C₁₋₄ aliphatic residue, or a N(C₁₋₄ aliphatic residue)-S(═O)₂—C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted;
R⁵⁰⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted;
on the condition that if R⁵⁰⁷ denotes a 3 to 10 membered heterocycloaliphatic residue, the 3 to 10 membered heterocycloaliphatic residue is linked via a carbon atom,
in which an “aliphatic group” and “aliphatic residue” can in each case be branched or unbranched, saturated or unsaturated,
in which a “cycloaliphatic residue” and a “heterocycloaliphatic residue” can in each case be saturated or unsaturated,
in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), a NH—C(═O)—C₁₋₄ aliphatic residue, a NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;
in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂—C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂;
in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂, 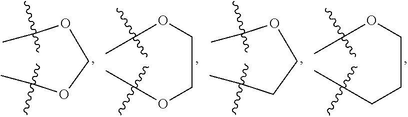 [SMILES: *OCO* ; *OCCO* ; *CCO* ; *CCCO*]
an NH(C₁₋₄ aliphatic residue), an N(C₁₋₄ aliphatic residue)₂, an NH—C(═O)—C₁₋₄ aliphatic residue, an NH—S(═O)₂—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂—C₁₋₄-aliphatic residue, a S(═O)₂—O—C₁₋₄-aliphatic residue, a S(═O)₂—NH—C₁₋₄-aliphatic residue, CN, CF₃, C(═O)H, C(═O)OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂.

2. The method according to claim 1, wherein:
R⁵⁰¹ represents the partial structure (5-T1),
—(CR^{508a}R^{508b})ₘ₅—R⁵⁰⁸ (5-T1),
wherein
m5 is 0, 1 or 2 and
R^{508a} and R^{508b} each independently of one another represent H, F, a O—C₁₋₄ aliphatic residue or a C₁₋₄ aliphatic residue;
R⁵⁰⁸ denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue,
or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or
wherein
m5 is 0,
R^{508a} and R^{508b} each independently of one another represent H, F, a O—C₁₋₄ aliphatic residue or a C₁₋₄ aliphatic residue; and
R^{508c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl,
wherein phenyl may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅,
X⁵ represents O or S;
R⁵⁰² is selected from the group consisting of F; Cl; Br; CF₃; CH₃; C₂H₅, iso-propyl; cyclopropyl; and O—CH₃;
R⁵⁰³, R⁵⁰⁴, R⁵⁰⁵ and R⁵⁰⁶ are each independently of one another selected from the group consisting of H; F; Cl; Br; CF₃; CN; OCF₃ and NO₂;
R⁵⁰⁷ denotes a C₁₋₆-aliphatic residue, unsubstituted or mono- or polysubstituted with at least one substituent selected from the group consisting of F, Cl, Br, I, OH, an O—C₁₋₄-aliphatic residue, COOH, a C(═O)—O—C₁₋₄-aliphatic residue, OCF₃, SH, SCF₃, a S—C₁₋₄-aliphatic residue, CF₃, and a C₁₋₄-aliphatic residue,
wherein the C₁₋₄-aliphatic residue in each case may be unsubstituted or mono- or polysubstituted with at least one substituent selected from the group consisting of OH, and an unsubstituted O—C₁₋₄-aliphatic residue,
or denotes an unsubstituted C₃₋₆-cycloaliphatic residue or an unsubstituted 3 to 6 membered heterocycloaliphatic residue,
on the condition that if R⁵⁰⁷ denotes a 3 to 6 membered heterocycloaliphatic residue, the 3 to 6 membered heterocycloaliphatic residue is linked via a carbon atom.
